(19) United States
(12) Patent Application Publication
(10) Pub. No.: US 2004/0266826 A1
(43) Pub. Date: Dec. 30, 2004

(54) Thiophenylthiopyrane dioxides as mmp or tnf-alpha inhibitors

(76) Inventors: Masahiro Neya, Osaka (JP); Akihiko Sawada, Osaka (JP); Kazuhiko Ohne, Osaka (JP); Yoshito Abe, Osaka (JP); Tsuyoshi Mizutani, Osaka (JP); Naoki Ishibashi, Osaka (JP); Makoto Inoue, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/487,242

(22) PCT Filed: Sep. 2, 2002
(86) PCT No.: PCT/JP02/08895

(30) Foreign Application Priority Data
Sep. 7, 2001 (AU) PR 7555

(51) Int. Cl. C 07D 4 9/04; A61K 31/453

(52) U.S. Cl. 514/326; 546/207

(57) ABSTRACT

A compound of the formula: in which R¹ is optionally substituted phenyl, optionally substituted naphthyl, optionally substituted bicyclic heterocyclic group, optionally substituted lower alkenyl or optionally substituted lower alkynyl, and R² is carboxy or protected carboxy, or a salt thereof, useful for treating and/or preventing MMP- or TNF-α mediated diseases.

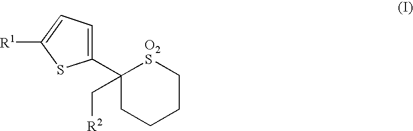

### FIELD OF THE INVENTION

The present invention relates to new compounds and pharmaceutically acceptable salts thereof.

More particularly, it relates to new compounds and pharmaceutically acceptable salts thereof which are useful as inhibitors of matrix metalloproteinases (hereinafter to be referred to as MMP) or the production of tumor necrosis factor α (hereinafter to be referred to as TNF α), to pharmaceutical compositions comprising the same, to use of the same as medicaments, and to methods for using the same therapeutically in the treatment and/or the prevention of MMP-or TNF α-mediated diseases.

### BACKGROUND ART

Some compounds to be useful as metalloproteinase inhibitors, or the like are known (WO 98/27069, WO 00/40576, WO 00/63165, WO01/60808, WO 02/30832, etc.).

### DISCLOSURE OF THE INVENTION

One object of the present invention is to provide new and useful cyclic compounds and pharmaceutically acceptable salts thereof, and to provide a process for preparing said new cyclic compound and salts thereof, which have pharmacological activities such as MMP-or TNF α-inhibitory activity and the like.

Another object of the present invention is to provide a pharmaceutical composition comprising, as an active ingredient, said cyclic compound or a pharmaceutically acceptable salt thereof.

A further object of the present invention is to provide use of said cyclic compounds and pharmaceutically acceptable salts thereof as medicaments for prophylactic and therapeutic treatment of MMP-or TNF α-mediated diseases.

A still further object of the present invention is to provide a method for using the same for the treatment and/or the prevention of MMP-or TNF α-mediated diseases in mammals, especially humans.

There are a number of structurally related metalloproteases which effect the breakdown of structural proteins. Matrix-degrading metalloproteases, such as gelatinase (MMP-2, MMP-9), stromelysin (MMP-3) and collagenase (MMP-1, MMP-8, MMP-13), are involved in tissue matrix degradation and have been implicated in many pathological conditions involving abnormal connective tissue and basement membrane matrix metabolism, such as arthritis (e.g., osteoarthritis and rheumatoid arthritis, etc.), cerebral disease (e.g., stroke, etc.), tissue ulceration (e.g., corneal, epidermal and gastric ulcerations, etc.), abnormal wound healing, periodontal disease, bone disease (e.g., Paget's disease and osteoporosis, etc.), tumor metastasis or invasion and HIV-infection, and also they have been implicated in many pathological conditions involving inframmatory respiratory diseases, for example, diseases or conditions such as chronic obstructive pulmonary disease (COPD); bronchial asthma; diseases associated with lung tissue injury and fibrosis, which occur from various causes, such as bronchitis, interstitial pneumonia, pulmonary fibrosis, acute respiratory distress syndrome (ARDS), etc.; rhinitis and sinusitis including allergic rhinitis and chronic sinusitis; and the like.

A tumor necrosis factor is recognized to be involved in many infections and autoimmune diseases. Furthermore, it has been shown that TNF is the prime mediator of the inflammatory response seen in sepsis and septic shock.

Collagenases initiate the degradation of collagen in vertebrates and, in addition to their normal function in the metabolism of connective tissue and wound healing, they have been implicated to be involved in a number of pathological conditions such as joint destruction in rheumatoid arthritis, periodontal disease, corneal ulceration, tumor metastasis, osteoarthritis, decubitus restenosis after percutaneous transluminal coronary angiopsty, osteoporosis, psoriasis, chronic active hepatitis, autoimmune keratitis, inframmatory respiratory diseases, for example, diseases or conditions such as chronic obstructive pulmonary disease (COPD); bronchial asthma; diseases associated with lung tissue injury and fibrosis, which occur from various causes, such as bronchitis, interstitial pneumonia, pulmonary fibrosis, acute respiratory distress syndrome (ARDS), etc.; rhinitis and sinusitis including allergic rhinitis and chronic sinusitis; and the like, and therefore the compounds of the present invention are useful for treating and/or preventing such pathological conditions.

The compounds of the present invention have inhibitory activity on MMP or the production of TNF α, and are useful for the treatment and/or prevention of diseases such as stroke, arthritis, cancer, tissue ulceration, decubitus ulcer, restenosis, periodontal disease, epidermolysis bullosa, scleritis, psoriasis and other diseases characterized by matrix metalloproteinase activity, as well as AIDS, sepsis, septic shock and other diseases caused by the production of TNF α.

And further, the compounds of the present invention, is useful for inframmatory respiratory disease treatment, for example, diseases or conditions such as chronic obstructive pulmonary disease (COPD); bronchial asthma; diseases associated with lung tissue injury and fibrosis, which occur from various causes, such as bronchitis, interstitial pneumonia, pulmonary fibrosis, acute respiratory distress syndrome (ARDS), etc.; rhinitis and sinusitis including allergic rhinitis and chronic sinusitis; and the like.

The object compounds of the present invention are novel and can be represented by the following formula (I):

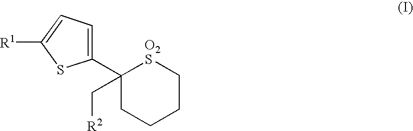

in which

R¹ is optionally substituted phenyl,

optionally substituted naphthyl,

optionally substituted bicyclic heterocyclic group

optionally substituted lower alkenyl or

optionally substituted lower alkynyl, and

R² is carboxy or protected carboxy, or a salt thereof.

Suitable salts of the object compound (I) may be conventional non-toxic pharmaceutically acceptable salts and include an acid addition salt such as an organic acid salt (e.g., acetate, trifluoroacetate, maleate, tartrate, fumarate, methanesulfonate, benzenesulfonate, formate, toluenesulfonate, etc.), an inorganic acid salt (e.g., hydrochloride, hydrobromide, hydroiodide, sulfate, nitrate, phosphate, etc.), or a salt with a base such as an amino acid (e.g., arginine, aspartic acid, glutamic acid, etc.), an alkali metal salt (e.g., sodium salt, potassium salt, etc.), an alkaline earth metal salt (e.g., calcium salt, magnesium salt, etc.), an ammonium salt, an organic base salt (e.g., trimethylamine salt, triethylamine salt, pyridine salt, picoline salt, dicyclohexylamine salt, N,N′-dibenzylethylenediamine salt, etc.), or the like. The object compounds and pharmaceutically acceptable salts thereof may include solvates such as enclosure compounds (e.g., hydrate, etc.).

The object compounds of the present invention can be prepared by the following processes.

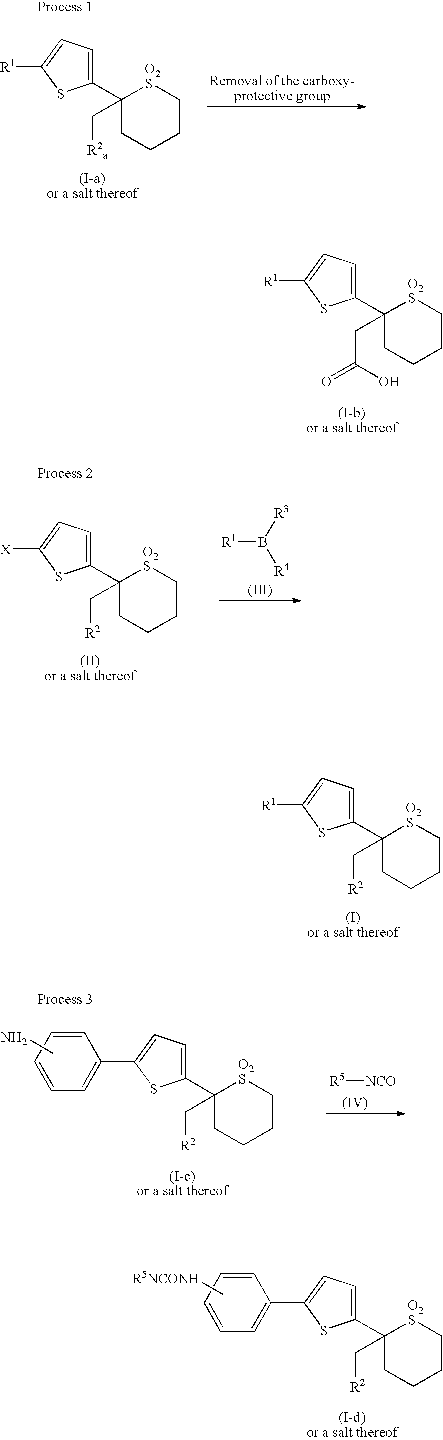

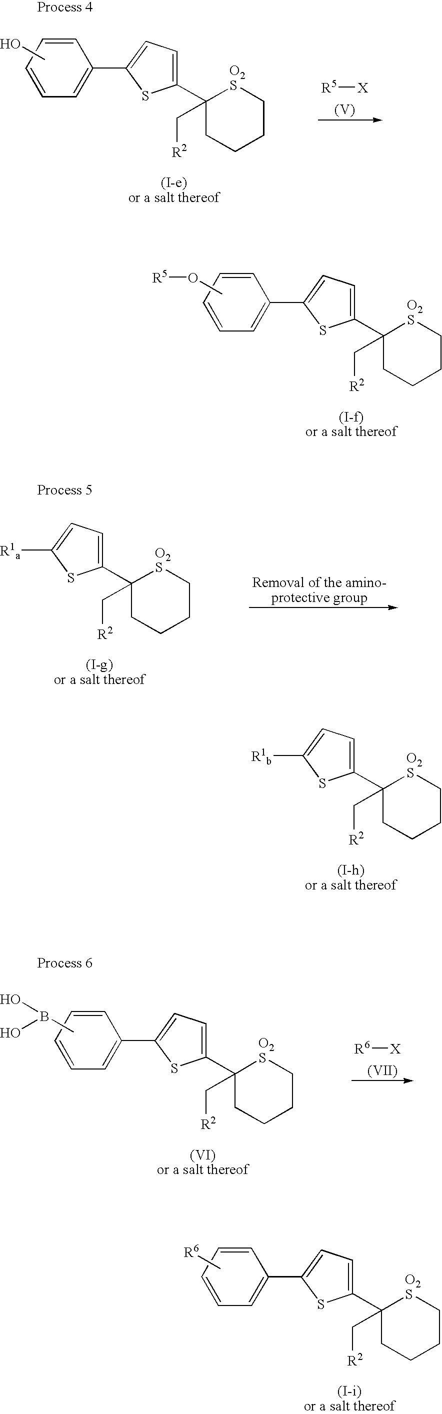

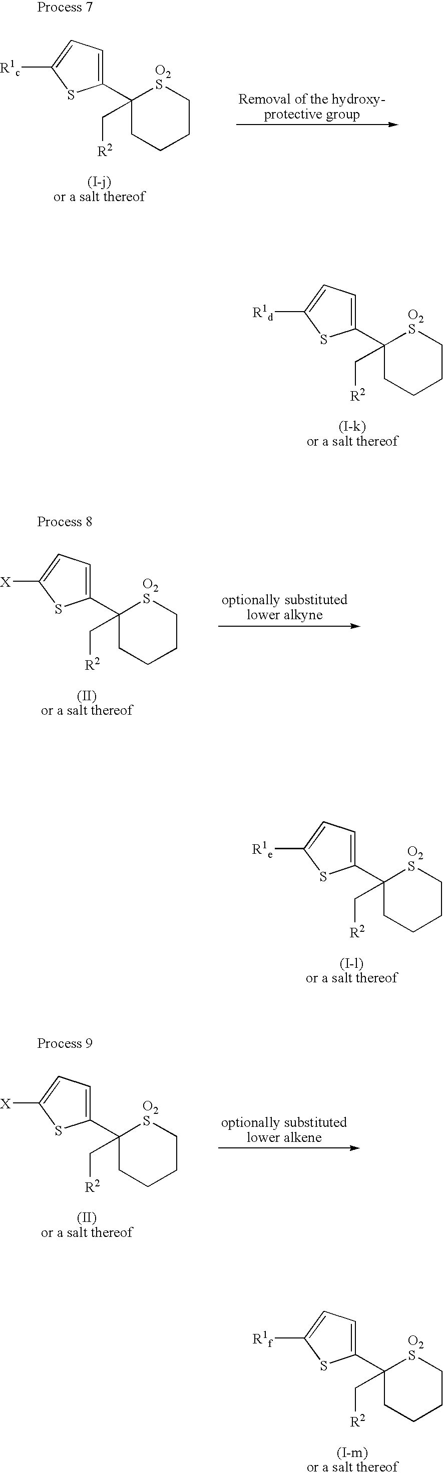

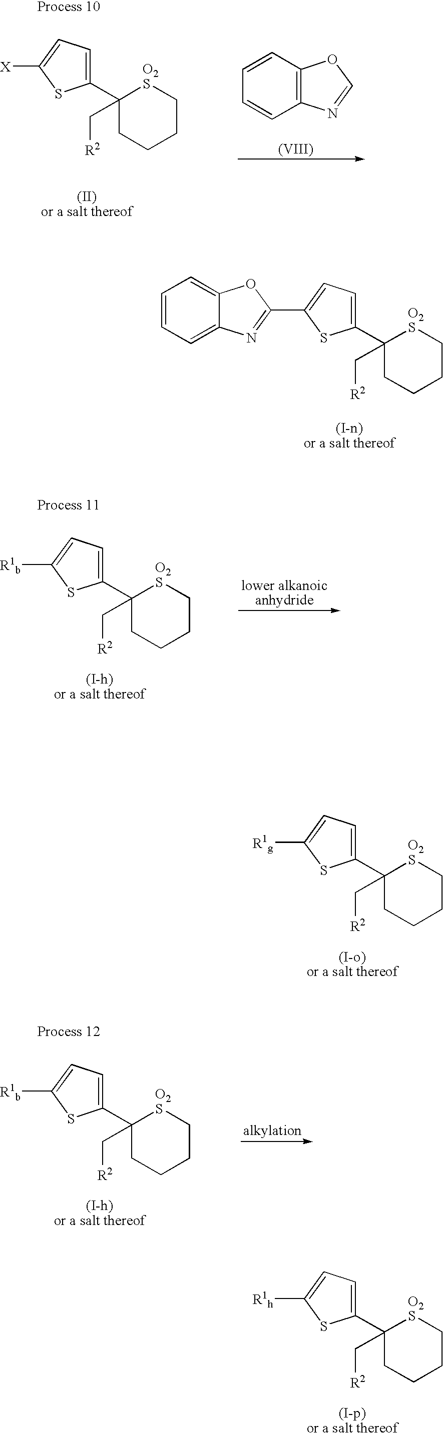

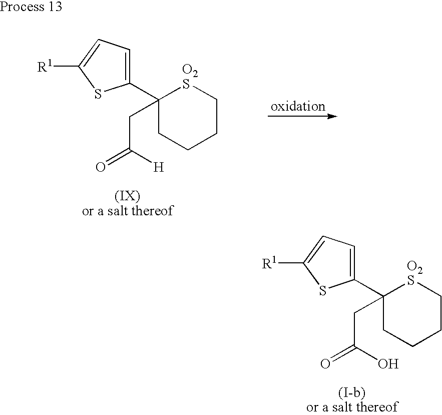

in which R¹ and R² are each as defined above,

Rₐ¹ is above-mentioned R¹, which

has a protected amino moiety such as lower alkoxycarbonylamino or lower alkanoyl amino moiety,

R_{b}¹ is above-mentioned R¹, which

has an amino moiety,

R_{c}¹ is above-mentioned R¹, which

has a protected hydroxy moiety such as lower alkoxycarbonyloxy moiety,

R_{d}¹ is above-mentioned R¹, which

has a hydroxy moiety,

Rₑ¹ is optionally substituted lower alkynyl,

R_{f}¹ is optionally substituted lower alkenyl,

R_{g}¹ is above-mentioned R¹, which

has an lower alkanoylamino moiety,

Rₙ¹ is above-mentioned R¹, which

has an mono- or di(lower)alkylamino moiety,

Rₐ² is protected carboxy,

R³ and R⁴ are each hydroxy, lower alkyl, or combined together to form lower alkylene,

R⁵ is lower alkyl,

R⁶ is suitable substituent, and

X is a leaving group.

The processes for preparing the object compounds are explained in detail in the following.

Process 1

The object compound (I-b) or a salt thereof can be prepared by subjecting a compound (I-a) or a salt thereof to removal reaction of the carboxy-protective group.

Suitable salts of the compounds (I-a) and (I-b) can be referred to the ones as exemplified for the compound (I).

This reaction is carried out in accordance with a conventional method such as solvolysis including hydrolysis, reduction or the like.

The solvolysis is preferably carried out in the presence of a base or an acid including Lewis acid.

Suitable base may include an inorganic base and an organic base such as an alkali metal [e.g. sodium, lithium, potassium, etc.], an alkaline earth metal [e.g. magnesium, calcium, etc.], the hydroxide or carbonate or bicarbonate thereof, hydrazine, trialkylamine [e.g. trimethylamine, triethylamine, etc.], picoline, 1,5-diazabicyclo[4.3.0]-non-5-ene, 1,4-diazabicyclo[2.2.2]octane, 1,8-diazabicyclo[5.4.0]undec-7-ene, or the like.

Suitable acid may include and organic acid [e.g. formic acid, acetic acid, propionic acid, trichloroacetic acid, trifluoroacetic acid, etc.], an inorganic acid (e.g. hydrochloric acid, hydrobromic acid, sulfuric acid, hydrogen chloride, hydrogen bromide, hydrogen fluoride, boron trifluoride diethyl etherate, hydrogen iodide, etc.].

The removal reaction using Lewis acid such as trihaloacetic acid [e.g. trichloroacetic acid, trifluoroacetic acid, etc.] or the like, is preferably carried out in the presence of cation trapping agents [e.g. anisole, phenol, etc.].

The reaction is usually carried out in a solvent such as water, an alcohol [e.g. methanol, ethanol, etc.], methylene chloride, chloroform, carbon tetrachloride, dioxane, tetrahydrofuran, N,N-dimethylformamide, a mixture thereof or any other solvent which does not adversely influence the reaction. A liquid base or acid can be also used as the solvent. The reaction temperature is not critical and the reaction is usually carried out under cooling to heating.

The reduction method applicable for the removal reaction may include chemical reduction and catalytic reduction.

Suitable reducing agents to be used in chemical reduction may include a combination of metal [e.g. tin, zinc, iron, etc.] or metallic compound [e.g. chromium chloride, chromium acetate, etc.] and an organic or inorganic acid [e.g. formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like, and these catalysts may be used in a combination with ammonium formate (e.g. a combination of palladium on carbon and ammonium formate, etc.).

The reduction is usually carried out in a conventional solvent which does not adversely influence the reaction such as water, methanol, ethanol, propanol, N,N-dimethylformamide, or a mixture thereof. Additionally, in case that the above-mentioned acid to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to heating.

Process 2

The compound (I) or a salt thereof can be prepared by reacting the compound (II) or a salt thereof with the compound (III).

Suitable salts of the compound (II) may be the same as those for the compound (I).

The reaction can be carried out in a conventional solvent such as water, acetone, dioxane, acetonitrile, 1,2-dimethoxyethane, chloroform, methylene chloride, ethylene chloride, tetrahydrofuran, ethyl acetate, N,N-dimethylformamide, pyridine and dichloromethane, a mixture thereof, or any other organic solvents which do not adversely affect the reaction.

This reaction can be carried out in the presence of an organic or inorganic base such as alkali metal (e.g. lithium, sodium, potassium, etc.), alkaline earth metal (e.g. calcium, etc.), alkali metal hydride (e.g. sodium hydride, etc.), alkaline earth metal hydride (e.g. calcium hydride, etc.), alkali metal hydroxide (e.g. sodium hydroxide, potassium hydroxide, etc.), alkali metal carbonate (e.g. sodium carbonate, potassium carbonate, etc.), alkali metal bicarbonate (e.g. sodium bicarbonate, potassium bicarbonate, etc.), alkali metal alkoxide (e.g. sodium methoxide, sodium ethoxide, potassium tert-butoxide, etc.), alkali metal alkanoic acid (e.g. sodium acetate, etc.), trialkylamine (e.g. triethylamine, etc.), pyridine compound (e.g. pyridine, lutidine, picoline, 4-dimethylaminopyridine, etc.), quinoline, lithium diisopropylamide, alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.), and the like.

The reaction is preferably carried out in the presence of a conventional condensing agent such as

N,N′-dicyclohexylcarbodiimide;

N-cyclohexyl-N′-morpholinoethylcarbodiimide;

N-cyclohexyl-N′-(4-diethylaminocyclohexyl)carbodiimide;

N,N′-diethylcarbodiimide; N,N′-diisopropylcarbodiimide;

N-ethyl-N′-(3-dimethylaminopropyl)carbodiimide;

N,N′-carbonylbis-(2-methylimidazole);

pentamethyleneketene-N-cyclohexylimine;

diphenylketene-N-cyclohexylimine; ethoxyacetylene;

1-alkoxy-1-chloroethylene; trialkyl phosphite;

ethyl polyphosphate; isopropyl polyphosphate; phosphorus

oxychloride (phosphoryl chloride);

phosphorus trichloride; diphenyl phosphorylazide;

thionyl chloride; oxalyl chloride; lower alkyl haloformate (e.g. ethyl chloroformate, isopropyl chloroformate);

triphenylphosphine;

tetrakis(triphenylphosphine)palladium(0);

2-ethyl-7-hydroxybenzisoxazolium salt; 2-ethyl-5-(m-sulfophenyl)isoxazolium hydroxide intramolecular salt;

1-(p-chlorobenzenesulfonyloxy)-6-chloro-1H-benzotriazole;

1-hydroxybenzotriazole; or so-called Vilsmeier reagent prepared by the reaction of N,N-dimethylformamide with thionyl chloride, phosgene, trichloromethyl chloroformate, phosphorus oxychloride or oxalyl chloride.

The reaction temperature is not critical, and the reaction can be carried out under from warming to heating.

Process 3

The compound (I-d) or a salt thereof can be prepared by reacting the compound (I-c) or a salt thereof, with a compound (IV).

Suitable salts of the compounds (I-c) and (I-d) may be the same as those for the compound (I).

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, etc., or a mixture thereof.

The reaction temperature is not critical and the reaction is usually carried out under from cooling to heating.

Process 4

The compound (I-f) or a salt thereof can be prepared by reacting the compound (I-e) or a salt thereof, with a compound (V).

Suitable salts of the compounds (I-e) and (I-f) may be the same as those for the compound (I).

This reaction can be carried out in the presence of an organic or inorganic base such as alkali metal (e.g. lithium, sodium, potassium, etc.), alkaline earth metal (e.g. calcium, etc.), alkali metal hydride (e.g. sodium hydride, etc.), alkaline earth metal hydride (e.g. calcium hydride, etc.), alkali metal hydroxide (e.g. sodium hydroxide, potassium hydroxide, etc.), alkali metal carbonate (e.g. sodium carbonate, potassium carbonate, etc.), alkali metal bicarbonate (e.g. sodium bicarbonate, potassium bicarbonate, etc.), alkali metal alkoxide (e.g. sodium methoxide, sodium ethoxide, potassium tert-butoxide, etc.), alkali metal alkanoic acid (e.g. sodium acetate, etc.), trialkylamine (e.g. triethylamine, etc.), pyridine compound (e.g. pyridine, lutidine, picoline, 4-dimethylaminopyridine, etc.), quinoline, lithium diisopropylamide, alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.), and the like.

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, etc., or a mixture thereof.

The reaction temperature is not critical and the reaction is usually carried out under from cooling to heating.

Process 5

The compound (I-h) or a salt thereof can be prepared by subjecting the compound (I-g) or a salt thereof to a removal reaction of the amino-protective group.

Suitable salts of the compounds (I-g) and (I-h) may be the same as those for the compound (I).

This reaction can be carried out in substantially the same manner as that of Process 1.

Process 6

The compound (I-i) or a salt thereof, with the compound (VII).

Suitable salts of the compound (I-i) may be the same as those for the compound (I).

This reaction can be carried out in substantially the same manner as that of Process 2.

Process 7

The compound (I-k) or a salt thereof can be prepared by subjecting the compound (I-j) or a salt thereof to a removal reaction of the hydroxy-protective group.

Suitable salts of the compounds (I-j) and (I-k) may be the same as those for the compound (I).

This reaction can be carried out in substantially the same manner as that of Process 1.

Process 8

The compound (I-l) or a salt thereof can be prepared by reacting the compound (II) or a salt thereof, with optionally substituted lower alkyne.

Suitable salts of the compound (I-l) may be the same as those for the compound (I).

This reaction can be carried out in the presence of a suitable condensing agent such as dichlorobis(triphenylphosophine)palladium(II), palladium(II) acetate, triphenylphosphine, tri-o-tolylphosphine, cupper iodide, and the like.

This reaction can be carried out in the presence of an organic or inorganic base such as alkali metal (e.g. lithium, sodium, potassium, etc.), alkaline earth metal (e.g. calcium, etc.), alkali metal hydride (e.g. sodium hydride, etc.), alkaline earth metal hydride (e.g. calcium hydride, etc.), alkali metal hydroxide (e.g. sodium hydroxide, potassium hydroxide, etc.), alkali metal carbonate (e.g. sodium carbonate, potassium carbonate, etc.), alkali metal bicarbonate (e.g. sodium bicarbonate, potassium bicarbonate, etc.), alkali metal alkoxide (e.g. sodium methoxide, sodium ethoxide, potassium tert-butoxide, etc.), alkali metal alkanoic acid (e.g. sodium acetate, etc.), trialkylamine (e.g. triethylamine, etc.), pyridine compound (e.g. pyridine, lutidine, picoline, 4-dimethylaminopyridine, etc.), quinoline, lithium diisopropylamide, alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.), and the like.

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, dioxane, etc., or a mixture thereof.

The reaction temperature is not critical and the reaction is usually carried out under from cooling to heating.

Process 9

The compound (I-m) or a salt thereof can be prepared by reacting the compound (II) or a salt thereof, with optionally substituted lower alkene.

Suitable salts of the compound (I-m) may be the same as those for the compound (I).

This reaction can be carried out in the presence of a suitable condensing agent such as dichlorobis(triphenylphosophine)palladium (II), palladium(II) acetate, triphenylphosphine, tri-o-tolylphosphine, cupper iodide, and the like.

This reaction can be carried out in the presence of an organic or inorganic base such as alkali metal (e.g. lithium, sodium, potassium, etc.), alkaline earth metal (e.g. calcium, etc.), alkali metal hydride (e.g. sodium hydride, etc.), alkaline earth metal hydride (e.g. calcium hydride, etc.), alkali metal hydroxide (e.g. sodium hydroxide, potassium hydroxide, etc.), alkali metal carbonate (e.g. sodium carbonate, potassium carbonate, etc.), alkali metal bicarbonate (e.g. sodium bicarbonate, potassium bicarbonate, etc.), alkali metal alkoxide (e.g. sodium methoxide, sodium ethoxide, potassium tert-butoxide, etc.), alkali metal alkanoic acid (e.g. sodium acetate, etc.), trialkylamine (e.g. triethylamine, etc.), pyridine compound (e.g. pyridine, lutidine, picoline, 4-dimethylaminopyridine, etc.), quinoline, lithium diisopropylamide, alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.), and the like.

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, dioxane, etc., or a mixture thereof.

The reaction temperature is not critical and the reaction is usually carried out under from cooling to heating.

Process 10

The compound (I-n) or a salt thereof can be prepared by reacting the compound (II) or a salt thereof, with the compound (VIII).

Suitable salts of the compound (I-n) may be the same as those for the compound (I).

This reaction can be carried out in the presence of a suitable condensing agent such as dichlorobis(triphenylphosophine)palladium (II), palladium(II) acetate, triphenylphosphine, tri-o-tolylphosphine, cupper iodide, and the like.

This reaction can be carried out in the presence of an organic or inorganic base such as alkali metal (e.g. lithium, sodium, potassium, etc.), alkaline earth metal (e.g. calcium, etc.), alkali metal hydride (e.g. sodium hydride, etc.), alkaline earth metal hydride (e.g. calcium hydride, etc.), alkali metal hydroxide (e.g. sodium hydroxide, potassium hydroxide, etc.), alkali metal carbonate (e.g. sodium carbonate, potassium carbonate, etc.), alkali metal bicarbonate (e.g. sodium bicarbonate, potassium bicarbonate, etc.), alkali metal alkoxide (e.g. sodium methoxide, sodium ethoxide, potassium tert-butoxide, etc.), alkali metal alkanoic acid (e.g. sodium acetate, etc.), trialkylamine (e.g. triethylamine, etc.), pyridine compound (e.g. pyridine, lutidine, picoline, 4-dimethylaminopyridine, etc.), quinoline, lithium diisopropylamide, alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.), and the like.

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, dioxane, etc., or a mixture thereof.

The reaction temperature is not critical and the reaction is usually carried out under from cooling to heating.

Process 11

The compound (I-o) or a salt thereof can be prepared by reacting the compound (I-h) or a salt thereof, with lower alkanoic anhydride.

Suitable salts of the compound (I-o) may be the same as those for the compound (I).

The reaction is usually carried out in a conventional solvent which does not adversely influence the reaction such as water, acetone, dichloromethane, alcohol (e.g. methanol, ethanol, etc.), tetrahydrofuran, pyridine, N,N-dimethylformamide, dioxane, etc., or a mixture thereof.

The reaction temperature is not critical and the reaction is usually carried out under from cooling to heating.

Process 12

The compound (I-p) or a salt thereof can be prepared by alkylating the compound (I-h) or a salt thereof.

Suitable salts of the compound (I-p) may be the same as those for the compound (I).

This alkylation reaction may include a conventional alkylation reaction which can convert amino moiety to mono- or di(lower)alkylamino moiety, and/or alkylation reaction of amino such as the one described in following Preparations and/or Examples or the similar manners thereto.

Process 13

The compound (I-b) or a salt thereof can be prepared by oxidating the compound (IX) or a salt thereof.

This oxidation reaction may include a conventional oxidation reaction which can convert formyl moiety to carboxy moiety, and/or oxodation reaction of formyl moiety such as the one described in following Preparations and/or Examples or the similar manners thereto.

The compounds obtained above can be isolated and purified by a conventional method such as pulverization, recrystallization, column chromatography, reprecipitation and the like.

The object compounds can be transformed into their salts in a conventional manner.

It is to be noted that the object compounds may include one or more stereoisomers or optical isomers due to asymmetric carbon atoms, and all of such isomers and mixture thereof are included within the scope of this invention.

The starting compounds used in the above processes can be prepared according to the following Preparations or by a conventional method.

Suitable examples and illustrations of the various definitions, which the present invention includes within its scope and which are shown in the above and subsequent descriptions of the present specification, are as follows.

The term “lower” is intended to mean up to 6 carbon atoms, preferably up to 4 carbon atoms, unless otherwise indicated.

Suitable “optionally substituted phenyl” may include phenyl optionally substituted by the group consisting of

lower alkyl (e.g. 4-(n-propyl)phenyl, 4-(n-butyl)phenyl, 4-(n-pentyl)phenyl),

amino (e.g. 4-aminophenyl),

lower alkylureido (e.g. 4-(n-propylureido)phenyl),

hydroxy (e.g. 4-hydroxyphenyl),

lower alkoxy (e.g. 4-ethoxyphenyl, 4-(n-propoxy)phenyl, 4-(i-propoxy)phenyl, 4-(butoxy)phenyl, 4-(n-pentyloxy)phenyl),

lower cycloalkyl (e.g. 4-cyclohexylphenyl),

phenyl(lower)alkoxy (e.g. 4-benzyloxyphenyl),

heterocyclic(lower)alkoxy such as oxazolyl(lower)alkoxy (e.g. 4-(5-oxazolylmethoxy)phenyl),

lower alkylcarbamoyl(lower)alkenyl (e.g. 4-(2-methylcarbamoylethenyl)phenyl),

heterocyclic(lower)alkenyl such as oxazolyl(lower)alkenyl (e.g. 4-(2-(oxazol-5-yl)ethenyl)phenyl),

heterocyclic-carbonylamino such as oxazolylcarbonylamino (e.g. 4-(oxazol-5-ylcarbonylamino)phenyl),

oxazolyl (e.g. 4-(2- or 4- or 5-oxazolyl)phenyl) optionally substituted by lower alkyl (e.g. 4-(2- or 4-methyl-5-oxazolyl)phenyl),

isoxazolyl (e.g. 4-(5-isoxazolyl)phenyl),

oxadiazolyl optionally substituted by lower alkyl (e.g. 4-(5-methyl-1,2,4-oxadiazol-3-yl)phenyl),

thiazolyl (e.g. 4-(2- or 4-thiazolyl)phenyl) optionally substituted by lower alkyl (e.g. 2-methyl-4-thiazolylphenyl),

pyridyl (e.g. 4-(2- or 3- or 4-pyridyl)phenyl),

pyrazolyl optionally substituted by lower alkyl (e.g. 4-(1-methyl-5-pyrazolyl)phenyl),

pyrazinyl (e.g. 4-(2-pyrazinyl)phenyl),

pyrimidinyl (e.g. 2-(5-pyrimidinyl)phenyl),

tetrazolyl optionally substituted by phenyl (e.g. 4-(2-phenyl-1,2,3,4,-tetrazol-5-yl)phenyl),

thienyl (e.g. 4-(2- or 3-thienyl)phenyl),

phenyl (e.g. 4-phenylphenyl),

lower alkoxyphenyl (e.g. 4-(4-methoxyphenyl)phenyl, 4-(4-ethoxyphenyl)phenyl),

lower alkylphenyl (e.g. 4-(2- or 3- or 4-methylphenyl)phenyl, 4-(4-ethylphenyl)phenyl, 4-(4-butylphenyl)phenyl),

lower alkylthiophenyl (e.g. 4-(4-methylthiophenyl)phenyl),

cyanophenyl (e.g. 4-(3- or 4-cyanophenyl)phenyl),

lower alkanoylphenyl (e.g. 4-(4-acetylphenyl)phenyl),

halophenyl (e.g. 4-(4-chlorophenyl)phenyl, 4-(4-fluorophenyl)phenyl),

trihalo(lower)alkylphenyl (e.g. 4-(4-trifluoromethylphenyl)phenyl),

trihalo(lower)alkanoylphenyl (e.g. 4-(4-trifluoroacetylphenyl)phenyl),

aminophenyl (e.g. 4-(4-aminophenyl)phenyl),

mono or di(lower)alkylaminophenyl (e.g. 4-(4-dimethylaminophenyl)phenyl),

lower alkoxycarbonylaminophenyl (e.g. 4-(4-methoxycarbonylaminophenyl)phenyl, 4-(4-t-butoxycarbonylaminophenyl)phenyl),

lower alkanoylaminophenyl (e.g. 4-(4-acetylaminophenyl)phenyl),

lower alkylsulfonylaminophenyl (e.g. 4-(4-methylsulfonylaminophenyl)phenyl),

lower alkylureidophenyl (e.g. 4-(4-ethylureidophenyl)phenyl),

carbamoylphenyl (e.g. 4-(4-carbamoylphenyl)phenyl),

mono or di(lower)alkylcarbamoylphenyl (e.g. 4-(4-methylcarbamoylphenyl)phenyl, 4-(4-dimethylcarbamoylphenyl)phenyl),

lower alkylsulfonylcarbamoylphenyl (e.g. 4-(4-methylsulfonylcarbamoylphenyl)phenyl),

lower alkoxycarbonylphenyl (e.g. 4-(4-methoxycarbonylphnenyl)phenyl),

lower alkylaminosulfonylphenyl (e.g. 4-(4-methylaminosulfonylphenyl)phenyl),

hydroxyphenyl (e.g. 4-(4-hydroxyphenyl)phenyl),

hydroxy(lower)alkylphenyl (e.g. 4-(4-hydroxymethylphenyl)phenyl), and

heterocyclic-phenyl such as oxazolylphenyl (e.g. 4-(4-(5-oxazolyl)phenyl)phenyl).

Preferable examples of “suitable substituents” for R⁶ may include the same one as mentioned for “optionally substituted phenyl”.

Suitable “optionally substituted naphthyl” may include naphthyl (e.g. 2-naphthyl) optionally substituted by the group consisting of

hydroxy (e.g. 6-hydroxy-2-naphthyl),

lower alkoxy (e.g. 6-methoxy-2naphthyl, 6-ethoxy-2-naphthyl),

lower alkoxy(lower)alkoxy (e.g. 6-methoxymethoxy-2-naphthyl),

cyano (e.g. 6-cyano-2-naphthyl),

lower alkanoyl (e.g. 6-formyl-2-naphthyl),

lower alkoxycarbonyl (e.g. 6-methoxycarbonyl-2-naphthyl),

lower alkylcarbamoyl (e.g. 6-methylcarbamoyl-2-naphthyl), and

heterocyclic group such as oxazolyl (e.g. 6-(5-oxazolyl)-2-naphthyl).

Suitable “optionally substituted bicyclic heterocyclic group” may include;

benzofuryl (e.g. 2- or 5-benzofuryl),

dihydrobenzofuyl (e.g. 2,3-dihydro-5-benzofuryl),

dioxoindanyl (e.g. 1,3-dioxoindan-5-yl),

benzothienyl (e.g. 2- or 3- or 5-benzothienyl) optionally substituted by the group consisting of lower alkyl (e.g. 5-methyl-2-benzothienyl),

lower alkoxy (e.g. 5- or 6-methoxy-2-benzothienyl) and halogen (e.g. 5- or 6-fluoro-2-benzothienyl),

quinolyl (e.g. 3- or 6-quinolyl),

dihydroindolyl optionally substituted by lower alkyl and oxo (e.g. 1-mehtyl-2-oxo-2,3-dihydroindol-5-yl),

benzoxazolyl (e.g. benzoxazol-2-yl),

dihydobenzothiazolyl (e.g. 3-methyl-2-oxo-2,3-dihydrobenzothiazol-5-yl).

Suitable “optionally substituted lower alkenyl” may include lower alkenyl optionally substituted by

C₆-C₁₀ aryl such as phenyl or naphthyl (e.g. 2-phenylethenyl, 2-(2-naphthyl)ethenyl) which is optionally substituted by the group consisting of

lower alkyl (e.g. 2-(4-methylphenyl)ethenyl),

halogen (e.g. 2-(4-chlorophenyl)ethenyl, 2-(4-fluorophenyl)ethenyl),

hydroxy (e.g. 2-(4-hydroxyphenyl)ethenyl),

lower alkoxycarbonyloxy (e.g. 2-(4-(t-butoxycarbonyloxy)phenyl)ethenyl),

lower alkoxy (e.g. 2-(4-methoxyphenyl)ethenyl), and

lower alkoxycarbonyloxy (e.g. 2-(4-t-butoxycarbonyloxyphenyl)ethenyl).

Suitable “optionally substituted lower alkynyl” may include lower alkynyl (e.g. 1-pentynyl) optionally substituted by C₆-C₁₀ aryl such as phenyl or naphthyl (e.g. phenylethynyl) which is optionally substituted by the group consisting of

lower alkyl (e.g. 4-methylphenylethynyl),

lower alkoxy (e.g. 4-methoxyphenylethynyl, 4-ethoxyphenylethynyl),

halo (e.g. 4-chlorophenylethynyl),

cyano (e.g. 4-cyanophenylethynyl), and

heterocyclic group such as oxazolyl (e.g. 4-(oxazol-5-yl)phenylethynyl).

Suitable “lower alkyl” may include a straight or ranched alkyl having 1 to 6 carbon atoms, and exemplified by methyl, ethyl, propyl, isopropyl, butyl, isobutyl, tert-butyl, pentyl, hexyl and the like, and the most preferably n-propyl, n-butyl and n-pentyl for R¹.

Suitable “aryl” may include aryl group having 6 to 10 carbon atoms such as phenyl, tolyl, xylyl, cumenyl, naphthyl and the like, and the most preferably phenyl and naphthyl.

Suitable “lower alkylureido” means ureido group substituted by above lower alkyl, for example, methylureido, ethylureido, propylureido, isopropylureido, butylureido, isobutylureido, tert-butylv, pentylureido, hexylv and the like, and the most preferably n-propylureido for R¹.

Suitable “lower alkoxy” may include a straight or branched alkoxy having 1 to 6 carbon atoms, and exemplified by methoxy, ethoxy, propoxy, isopropoxy, butoxy, isobutoxy, tert-butoxy, pentyloxy, tert-pentyloxy, hexyloxy and the like, and the most preferably ethoxy, n-propoxy, 1-propoxy, n-butoxy, and n-pentyloxy for R¹.

Suitable “heterocyclic(lower)alkoxy” may include a straight or branched alkoxy having 1 to 6 carbon atoms substituted by heterocyclic group such as oxazolyl(lower)alkoxy, and the most preferable one may be 5-oxazolylmethoxy.

Suitable “lower alkylcarbamoyl(lower)alkenyl” may include a straight or branched alkenyl having 2 to 6 carbon atoms substituted by lower alkylcarbamoyl, and exemplified by methylcarbamoylvinyl, ethylcarbamoylvinyl, ethylcarbamoylpropenyl, methylcarbamoylbutenyl, methylcarbamoylpentenyl, and the like, and the most preferably 2-methylcarbamoylethenyl, and the like.

Suitable “lower alkenyl” may include a straight or branched alkenyl having 2 to 6 carbon atoms, and exemplified by ethenyl, 1-propenyl, 2-propenyl, 1-butenyl, 2-butenyl, 1-pentenyl, 2-pentenyl and the like and the most preferably ethenyl, and the like.

Suitable “lower alkynyl” may include a straight or branched alkynyl having 2 to 6 carbon atoms, and exemplified by ethynyl, 1-propynyl, 2-propynyl, 1-butynyl, 2-butynyl, 1-pentynyl, 2-pentynyl and the like and the most preferably ethynyl and 1-pentynyl.

Suitable “substituted aryl” may include C₆-C₁₀ aryl substituted by the group consisting of lower alkyl, lower alkoxy, halo, cyano and heterocyclic (e.g. oxazolyl), and eexplified by lower alkylphenyl, lower alkoxyphenyl, halophenyl, cyanophenyl, oxazolylphenyl, and the like, and the most preferably 4-methylphenyl, 4-methoxyphenyl, 4-ethoxyphenyl, 4-chlorophenyl, 4-cyanophenyl, 4-(oxazol-5-yl)phenyl.

Suitable “lower alkylcarbamoyl” may include a straight or branched alkyl having 1 to 6 carbon atoms, and exemplified by methylcarbamoyl, ethylcarbamoyl, propylcarbamoyl, isopropylcarbamoyl, butylcarbamoyl, isobutylcarbamoyl, tert-butylcarbamoyl, pentylcarbamoyl, hexylcarbamoyl, and the like, and the most preferably methylcarbamoyl.

Suitable “heterocyclic(lower)alkenyl” may include lower alkenyl as mentioned above, which is substituted by heterocyclic group such as oxazolyl(lower)alkenyl, and the most preferable one may be 2-(oxazol-5-yl)ethenyl.

Suitable “heterocyclic-carbonylamino” may include amino group substituted by heterocyclic-carbonyl group such as oxazolylcarbonylamino, and the most preferable one may be oxazol-5-ylcarbonylamino.

Suitable “phenyl(lower)alkoxy” means above lower alkoxy, substituted by phenyl, and exemplified by benzyloxy, phenylethoxy, phenylpropoxy, phenylisopropoxy, phenylbutoxy, phenylisobutoxy, phenyl-tert-butoxy, phenylpentyloxy, phenyl-tert-pentyloxy, phenylhexyloxy and the like, and the most preferably benzyloxy for R¹.

Suitable “lower cycloalkyl” may include C₃-C₆ cycloalkyl such as cyclopropyl, cyclobuty, cyclopropyl, cyclobutyl, cyclopentyl, cyclohexylcarbamoyl, and the like, and the most preferably cyclohexyl for R¹.

Suitable “lower alkoxyphenyl” means phenyl substituted by above lower alkoxy, and exemplified by methoxyphenyl, ethoxyphenyl, propoxyphenyl, isopropoxyphenyl, butoxyphenyl, isobutoxyv, tert-butoxyphenyl, pentyloxyphenyl, tert-pentyloxyphenyl, hexyloxyphenyl and the like, and the most preferably methoxyphenyl and ethoxyphenyl for R¹.

Suitable “lower alkylphenyl” may include methylphenyl, ethylphenyl, propylhenyl, butylphenyl, and the like, and the most preferable one may be 4-methylphenyl, 4-ethylphenyl, 4-n-butylphenyl.

Suitable “lower alkylthiophenyl” may include methylthiophenyl, ethylthiophenyl, propylthiohenyl, butylthiophenyl, and the like, and the most preferable one may be 4-methylthiophenyl.

Suitable “lower alkanoylphenyl” may include formylphenyl, acetylphenyl, propionylhenyl, butyrylphenyl, and the like, and the most preferable one may be 4-acetylphenyl.

Suitable “halophenyl” may include chlorophenyl, fluorophenyl, bromophenyl, iodophenyl, and the like, and the most preferable one may be 4-clorophenyl, 4-fluorophenyl.

Suitable “trihalo(lower)alkylphenyl” may include phenyl group substituted by trihalogenated lower alkyl such as trifluoromethyl, trifluoroethyl, and the like, and the most preferably 4-trifluoromethylphenyl.

Suitable “trihalo(lower)alkanoylphenyl” may include phenyl group substituted by trihalogenated lower alkanoyl such as trifluoroacetyl, trifluoroethyl, and the like, and the most preferably 4-trifluoroacetylphenyl.

Suitable “mono- or di(lower)alkylaminophenyl” may include phenyl group substituted by mono- or di(lower)alkylamino such as methylamino, ethylamino, propylamino, butylamino, dimethylamino, diethylamino, N-methyl-N-ethylamino, and the like, and the most preferably 4-dimethylaminophenyl.

Suitable “heterocyclic-phenyl” may include phenyl group substituted by heterocyclic group such as oxazolylphenyl, and most preferably 4-(5-oxazolyl)phenyl.

Preferable example of “naphthyl substituted by heterocyclic group” may include oxazolylnaphthyl, and the like, and the most preferably 6-(5-oxazolyl)-2-naphthyl.

Suitable “heterocyclic group” may include saturated or unsaturated, 3 to 8-membered monocyclic heterocyclic group, or 7 to 13-membered bicyclic-heterocyclic group, containing at least one hetero atom such as oxygen atom, sulfur atom, nitrogen atom and the like.

Suitable example of “monocyclic heterocyclic group” may include;

(1) unsaturated 3 to 8-membered (more preferably 5 or 6-membered) heteromonocyclic group containing 1 to 4 nitrogen atom(s), for example,

pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), etc.;

(2) saturated 3 to 8-membered (more preferably 5 or 6-membered) heteromonocyclic group containing 1 to 4 nitrogen atom(s), for example,

pyrrolidinyl, imidazolidinyl, piperidyl, piperazinyl, etc.;

(3) unsaturated 3 to 8-membered (more preferably 5 or 6-membered) heteromonocyclic group containing 1 or 2 oxygen atom(s) and 1 to 3 nitrogen atom(s), for example,

oxazolyl, isoxazolyl, oxadiazolyl (e.g., 1,2,4-oxadiazolyl, 1,3,4-oxadiazolyl, 1,2,5-oxadiazolyl, etc.), etc.;

(4) saturated 3 to 8-membered (more preferably 5 or 6-membered) heteromonocyclic group containing 1 or 2 oxygen atom(s) and 1 to 3 nitrogen atom(s), for example,

morpholinyl, sydnonyl, etc.;

(5) unsaturated 3 to 8-membered (more preferably 5 or 6-membered) heteromonocyclic group containing 1 or 2 sulfur atom(s) and 1 to 3 nitrogen atom(s), for example,

thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;

(6) saturated 3 to 8-membered (more preferably 5 or 6-membered) heteromonocyclic group containing 1 or 2 sulfur atom(s) and 1 to 3 nitrogen atom(s), for example,

thiazolidinyl, etc.;

(7) unsaturated 3 to 8-membered (more preferably 5 or 6-membered) heteromonocyclic group containing 1 or 2 sulfur atom(s), for example,

thienyl, dihydrodithiinyl, dihydrodithionyl, etc.;

(8) unsaturated 3 to 8-membered (more preferably 5 or 6-membered) heteromonocyclic group containing an oxygen atom, for example,

furyl, etc.;

(9) saturated 3 to 8-membered (more preferably 5 or 6-membered) heteromonocyclic group containing an oxygen atom, for example,

tetrahydrofuran, tetrahydropyran, etc.;

(10) unsaturated 3 to 8-membered (more preferably 5 or 6-membered) heteromonocyclic group containing an oxygen atom and 1 or 2 sulfur atom(s), for example,

dihydrooxathiinyl, etc.; and the like.

Suitable example of “bicyclic heterocyclic group” may include;

(11) unsaturated bicyclic 7- to 13-membered, preferably 9- or 10-membered, heterocyclic group containing 1 to 5 nitrogen atoms, for example,

indolyl, dihydroindolyl, isoindolyl, indolizinyl, benzimidazolyl, quinolyl, isoquinolyl, indazolyl, benzotriazolyl, tetrazolopyridyl, tetrazolopyridazinyl (e.g., tetrazolo[1,5-b]pyridazinyl, etc.), dihydrotriazolopyridazinyl, and the like;

(12) unsaturated bicyclic 7- to 13-membered, preferably 9- or 10-membered, heterocyclic group containing 1 or 2 oxygen atoms and 1 to 3 nitrogen atoms, for example,

benzoxazolyl, benzoxadiazolyl, and the like;

(13) unsaturated bicyclic 7- to 13-membered, preferably 9- or 10-membered, heterocyclic group containing 1 or 2 sulfur atoms and 1 to 3 nitrogen atoms, for example,

benzothiazolyl, dihydrobenzothiazolyl, benzothiadiazolyl, and the like;

(14) unsaturated bicyclic 7- to 13-membered, preferably 9- or 10-membered, heterocyclic group containing 1 or 2 oxygen atoms, for example,

benzofuranyl, dihydrobenzofuranyl, benzodioxolenyl (dioxaindan), and the like;

(15) unsaturated bicyclic 7- to 13-membered, preferably 9- or 10-membered, heterocyclic group containing 1 or 2 sulfur atoms, for example,

benzothiophenyl, dihydrobenzothiophenyl, and the like.

These heterocyclic groups may be substituted by the suitable substituents such as lower alkyl, oxo, lower alkoxy, halogen, phenyl, and the like.

More preferable examples of the heterocyclic group thus defined may be:

(1) unsaturated 5 or 6-membered heteromonocyclic group containing 1 to 4 nitrogen atom(s),

(3) unsaturated 5 or 6-membered heteromonocyclic group containing 1 or 2 oxygen atom(s) and 1 to 3 nitrogen atom(s),

(5) unsaturated 3 to 8-membered (more preferably 5 or 6-membered) heteromonocyclic group containing 1 or 2 sulfur atom(s) and 1 to 3 nitrogen atom(s),

(7) unsaturated 3 to 8-membered (more preferably 5 or 6-membered) heteromonocyclic group containing 1 or 2 sulfur atom(s),

(11) unsaturated bicyclic 9- or 10-membered, heterocyclic group containing 1 to 5 nitrogen atoms,

(12) unsaturated bicyclic 9- or 10-membered, heterocyclic group containing 1 or 2 oxygen atoms and 1 to 3 nitrogen atoms,

(13) unsaturated bicyclic 9- or 10-membered, heterocyclic group containing 1 or 2 sulfur atoms and 1 to 3 nitrogen atoms,

(14) unsaturated bicyclic 9- or 10-membered, heterocyclic group containing 1 or 2 oxygen atoms,

(15) unsaturated bicyclic 9- or 10-membered, heterocyclic group containing 1 or 2 sulfur atoms, and the like;

wherein these heterocyclic groups may be substituted by the group consisting of lower alkyl, oxo, lower alkoxy, halogen, and phenyl;

and the most preferable one may be

(1) pyrazolyl (e.g. 5-pyrazolyl), lower alkylpyrazolyl (e.g. 1-methylpyrozol-5-yl), pyridyl (e.g. 2- or 3- or 4-pyridyl), pyrimidyl (e.g. 5-pyrimidinyl), pyrazinyl (e.g. 2-pyrazinyl), tetrazolyl, phenyltetrazolyl (e.g. 2-phenyl-2H-tetrazol-5-yl);

(3) oxazolyl (e.g. 2- or 4- or 5-oxazolyl), lower alkyloxazolyl (e.g. 2- or 4-methyl-5-oxazolyl), isoxazolyl (e.g. 5-isoxazolyl), oxadiazolyl, lower alkyloxadiazolyl (e.g. 5-methyl-1,2,4-oxadiazol-3-yl);

(5) thiazolyl (e.g. 2- or 4-thiazolyl), lower alkylthizazolyl (e.g. 2-methylthiazol-4-yl);

(7) thienyl (e.g. 2- or 3-thienyl);

(11) indolyl, dihydroindolyl, dihydroindolyl substituted by lower alkyl and oxo (e.g. 1-mehtyl-2-oxo-2,3-dihydroindol-5-yl), quinolyl (e.g. 3- or 6-quinolyl);

(12) benzoxazolyl (e.g. benzoxazol-2-yl);

(13) dihydrobenzothiazolyl, dihydrobenzothiazolyl substituted by lower alkyl and oxo (e.g. 3-methyl-2-oxo-2,3-dihydrobenzothiazol-5-yl);

(14) benzofuranyl (e.g. 2- or 5-benzofuryl), dihydrobenzofuranyl (e.g. 2,3-dihydro-5-benzofuryl), benzodioxolenyl (e.g. 1,3-dioxoindan-5-yl);

(15) benzothiophenyl (e.g. 2- or 3- or 5-benzothiophenyl), lower alkylbenzothiophenyl (e.g. 5-methyl-2-benzothiophenyl), lower alkoxybenzothiophenyl (e.g. 5- or 6-methoxy-2-benzothiophenyl), halobenzothiophenyl (e.g. 5- or 6-fluoro-2-benzothiophenyl).

Suitable “protected carboxy” includes esterified carboxy wherein “esterified carboxy” is as defined below.

Suitable examples of the ester moiety of the esterified carboxy are lower alkyl ester (e.g., methyl ester, ethyl ester, propyl ester, isopropyl ester, butyl ester, isobutyl ester, tert-butyl ester, pentyl ester, hexyl ester, etc.) and the like, which may have at least one suitable substituent. Examples of the substituted lower alkyl ester are lower alkanoyloxy(lower)alkyl ester [e.g., acetoxymethyl ester, propionyloxymethyl ester, butyryloxymethyl ester, valeryloxymethyl ester, pivaloyloxymethyl ester, hexanoyloxymethyl ester, 1-(or 2-)acetoxyethyl ester, 1-(or 2- or 3-)acetoxypropyl ester, 1-(or 2- or 3- or 4-)acetoxybutyl ester, 1-(or 2-)-propionyloxyethyl ester, 1-(or 2- or 3-)propionyloxypropyl ester, 1-(or 2-)butyryloxyethyl ester, 1-(or 2-)-isobutyryloxyethyl ester, 1-(or 2-)pivaloyloxyethyl ester, 1-(or 2-)hexanoyloxyethyl ester, isobutyryloxymethyl ester, 2-ethylbutyryloxymethyl ester, 3,3-dimethylbutyryloxymethyl ester, 1-(or 2-)pentanoyloxyethyl ester, etc.], lower alkanesulfonyl(lower)alkyl ester (e.g., 2-mesylethyl ester, etc.), mono(or di or tri)halo(lower)alkyl ester (e.g., 2-iodoethyl ester, 2,2,2-trichloroethyl ester, etc.); trimethysilyl(lower)alkyl ester (e.g. 2-trimethylsilylethyl ester, etc.); lower alkoxycarbonyloxy(lower)alkyl ester [e.g., methoxycarbonyloxymethyl ester, ethoxycarbonyloxymethyl ester, propoxycarbonyloxymethyl ester, tert-butoxycarbonyloxymethyl ester, 1-(or 2-)methoxycarbonyl-oxyethyl ester, 1-(or 2-)ethoxycarbonyloxyethyl ester, 1-(or 2-)isopropoxycarbonyloxyethyl ester, etc.], phthalidylidene(lower)alkyl ester, (5-lower alkyl-2-oxo-1,3-dioxol-4-yl)(lower)alkyl ester [e.g., (5-methyl-2-oxo-1,3-dioxol-4-yl)methyl ester, (5-ethyl-2-oxo-1,3-dioxol-4-yl)methyl ester, (5-propyl-2-oxo-1,3-dioxol-4-yl)ethyl ester, etc.]; lower alkenyl ester (e.g., vinyl ester, allyl ester, etc.); lower alkynyl ester (e.g., ethynyl ester, propynyl ester, etc.); ar(lower)alkyl ester which may have at least one suitable substituent (e.g., benzyl ester, 4-methoxybenzyl ester, 4-nitrobenzyl ester, phenethyl ester, trityl ester, benzhydryl ester, bis(methoxyphenyl)methyl ester, 3,4-dimethoxybenzyl ester, 4-hydroxy-3,5-di-tert-butylbenzyl ester, etc.); aryl ester which may have at least one suitable substituent (e.g., phenyl ester, 4-chlorophenyl ester, tolyl ester, tert-butylphenyl ester, xylyl ester, mesityl ester, cumenyl ester, etc.); phthalidyl ester; and the like.

More preferable example of the protected carboxy thus defined may be trimethylsilyl(lower)alkoxycarbonyl, lower alkoxycarbonyl and lower alkoxyphenyl(lower)alkoxycarbonyl, and the most preferable one may be 2-trimethylsilylethoxycarbonyl, t butoxycarbonyl, and p-methoxybenzyloxycarbonyl.

Suitable “leaving group” may include halo (e.g. chloro, bromo, fluoro, iodo, etc.), acyloxy such as sulfonyloxy (e.g., mesyloxy, tosyloxy, etc.), alkoxy (e.g., tert-butoxy, etc.), aralkoxy (e.g., benzyloxy, etc.), and the like, preferably halo and the most preferably bromo and iodo.

Preferable examples of the compound (I) is; (1) a compound of the formula:

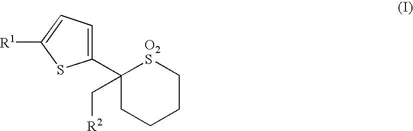

in which R¹ is phenyl substituted by the group consisting of lower alkyl (e.g. butyl, pentyl, etc.), amino, lower alkylureido (e.g. propylureido, etc.), hydroxy, lower alkoxy (e.g. propoxy, etc.), lower cycloalkyl (e.g. cyclohexyl, etc.), phenyl(lower)alkoxy (e.g. benzyloxy, etc.), oxazolyl, phenyl, lower alkoxyphenyl (e.g. ethoxyphenyl, etc.) and cyanophenyl; naphthyl; or bicyclic heterocyclic group (e.g. quinolyl, benzofuranyl, dihydrobenzofuranyl, benzodioxolenyl, benzothiophenyl, etc.), and

R² is carboxy or protected carboxy (e.g. trimethylsilyl(lower)alkoxycarbonyl, etc.),

or a salt thereof; preferably

(2) the compound of above (1), wherein

R¹ is lower alkylphenyl (e.g. butylphenyl, pentylphenyl, etc.), aminophenyl, lower alkylureidophenyl (e.g. propylureidophenyl, etc.), hydroxyphenyl, lower alkoxyphenyl (e.g. propoxyphenyl, etc.), lower cycloalkylphenyl (e.g. cyclohexylphenyl, etc.), phenyl(lower)alkoxyphenyl (e.g. benzyloxyphenyl, etc.), oxazolylphenyl, biphenylyl, lower alkoxybiphenylyl (e.g. ethoxybiphenylyl, etc.), cyanobiphenylyl, naphthyl or bicyclic heterocyclic group (e.g. quinolyl, benzofuranyl, dihydrobenzofuranyl, benzodioxolenyl, benzothiophenyl, etc.), and

R² is carboxy or protected carboxy (e.g. trimethylsilyl(lower)alkoxycarbonyl, etc.),

or a salt thereof; and more preferably

(3) the compound of above (2), wherein

R¹ is butylphenyl (e.g. 4-butylphenyl, etc.), pentylphenyl (4-n-pentylphenyl, etc.), propylureidophenyl (e.g. 3-(n-propyliureido)-phenyl, etc.), propoxyphenyl (e.g. 3-propoxyphenyl, etc.), cyclohexylphenyl (e.g. 4-cyclohexylphenyl, etc.), benzyloxyphenyl (e.g. 4-benzyloxyphenyl, etc.), oxazolylphenyl (e.g. 4-(1,3-oxazol-2-(or 4- or 5-)yl)phenyl, etc.), biphenylyl(e.g. 4-biphenylyl, etc.), ethoxybiphenylyl (e.g. 4′-ethoxy-4-biphenylyl, etc.), cyanobiphenylyl (e.g. 4′-cyano-4-biphenylyletc.), naphthyl (e.g. 2-naphthyl, etc.), quinolyl (e.g. 3-(or 4-)-quinolyl, etc.), benzofuranyl (e.g. benzofuran-2-(or 5-)yl, etc.), dihydrobenzofuranyl (e.g. 2,3-dihydrobenzofuran-5-yl, etc.), benzodioxolenyl (e.g. 1,3-benzodioxolen-5-yl, etc.), or benzothiophenyl (e.g. benzothiophen-2-yl, etc.), and

R² is carboxy; and other preferable examples are

(4) the compounds as described in the claims.

The compounds of the present invention (I), where R² is carboxy, may be useful, iner alia, for inframmatory respiratory disease treatment.

Inhibitory activity of MMP can be assayed by a conventional test method as mentioned below.

Test Methods:

Test Method 1:

Inhibitory Activity of Human MMP-1

Human collagenase was prepared from the culture medium of human skin fibroblast stimulated with interleukin-1β (1 ng/ml). Latent collagenase was activated by incubation with tryspin (200 μg/ml) at 37° C. for 60 minutes and the reaction was stopped by adding soybean trypsin inhibitor (800 μg/ml). Collagenase activity was determined using FITC-labeled calf skin type I collagen. FITC-collagen (2.5 mg/ml) was incubated at 37° C. for 120 minutes with the activated collagenase and test compound in 50 mM Tris buffer (containing 5 mM CaCl₂, 200 mM NaCl and 0.02% NaN₃, pH 7.5). After stopping the enzyme reaction by adding the equal volume of 70% ethanol-200 mM Tris buffer (pH 9.5), the reaction mixture was centrifuged, and collagenase activity was estimated by measuring the fluorescence intensity of supernatant at 495 nm (excitation) and 520 nm (emission).

Test Method 2:

Inhibitory Activity of Human MMP-9

The inhibitory activity of test compounds against human MMP-9 were measured by using commercial kits (Yagai, Japan). Gelatinolytic activity was determined by monitoring the degradation of FITC-labeled bovine type IV collagen after incubation for 4 hours at 42° C. The amount of degraded collagen was estimated by measuring the fluorescence intensity at 495 nm (excitation) and 520 nm (emission).

Test Method 3

Inhibitory Activity of Human MMP-13

The inhibitory potential of test compounds against human MMP-13 were assayed by using commercial kit (Chondrex, USA) contained truncated form of human recombinant MMP-13 and fluorogenic peptide substrate. Activity of human MMP-13 was determined by monitoring the degradation of fluorogenic peptide substrate after incubation for 1 hour at 35° C. and estimated by measuring the fluorescence intensity of degraded peptide substrate at 495 nm (excitation) and 520 nm (emission).

Test Method 4:

Inhibitory Activity of Human MMP-8

The inhibitory potential of test compounds against human MMP-8 were assayed by using commercial kit (Chondrex, USA) contained recombinant human pro-MMP-8 and FITC-labeled telopeptide-free soluble bovine type I collagen as a substrate. Recombinant human pro-MMP-8 was activated by a sequential incubation with mercury compound and proteinase at 35° C. for 1 hour. Reaction mixture containing the activated MMP-8, substrate and test compounds were incubated at 35° C. for 2 hours. After stopping the enzyme reaction by adding the stop solution (o-phenathroline), the reaction mixture was centrifuged and MMP-8 activity was estimated by measuring the fluorescence intensity of supernatant at 490 nm (excitation) and 520 nm (emission).

Test Method 5:

Effect on Lipopolysaccharide (LPS)-Induced Lung Injury in Mice

Mice were anesthetized and injected LPS solution intratracheally. After 24 hours, mice were sacrificed and bronchoalveolar lavage (BAL) was repeated three times via a tracheal cannula. The BAL fluid was freeze-thawed and sonicated to lyse cells including erythrocytes. Hemoglobin content in the cell lysate was assayed by measuring optical density at 406 nm as a lung injury marker. Bovine hemoglobin was used to construct a standard curve. The test sompounds or the vehicle was given perorally (p.o.) four times, 24, 16 and 1 hour before plus 7 hours after the LPS injection.

For therapeutic purposes, the compounds and pharmaceutically acceptable salts thereof of the present invention can be used in the form of a pharmaceutical preparation containing, as an active ingredient, one of said compounds in admixture with a pharmaceutically acceptable carrier such as an organic or inorganic solid or liquid excipient suitable for oral, parenteral or external administration. The pharmaceutical preparations may be capsules, tablets, dragees, granules, solutions, suspensions, emulsions, sublingual tablets, suppositories, ointments, and the like. If desired, there may be included, in these preparations, auxiliary substances, stabilizing agents, wetting agents, emulsifying agents, buffers and other commonly used additives.

While the dose of the compound will vary depending upon the age and condition of patient and the like, in the case of intravenous administration, a daily dose of 0.01-100 mg of the active ingredient per kg weight of a human being, and in the case of intramuscular administration, a daily dose of 0.05-100 mg of the same per kg weight of a human being, or in the case of oral administration, a daily dose of 0.1-100 mg of the same per kg weight of a human being, is generally given for the treatment of MMP or TNF α-mediated diseases.

In order to illustrate the usefulness of the object compound, the pharmacological test data of a representative compound of the compounds are shown in the following.

Inhibitory Activity of MMP

1. Test Method

Inhibitory activity of human MMP-9 as mentioned above.

2. Test Compounds

Compound of Example 3, 35, 51, 97 and 179

3. Test Results

Test Results are shown in the following table.

| | Inhibitory activity |
|---|---|
| Test Compounds | [IC₅₀ (nM)) |
| Example 3 | 16.0 |
| Example 35 | 2.77 |
| Example 51 | 4.56 |
| Example 97 | 1.37 |
| Example 179 | 7.17 |

The following Preparations and Examples are given for the purpose of illustrating the present invention in detail.

Preparation 1

To a suspension of [(2S)-2-(5-bromothiophen-2-yl)-1,1-dioxo-3,4,5,6-tetrahydro-2H-thiopyran-2-yl]acetic acid (2.0 g) and 2-(trimethylsilyl)ethanol (1.62 ml) in dichloromethane (10 ml) were added 4-(dimethylamino)pyridine (69 mg) and 1-ethyl-3-(3-dimethylaminopropyl)carbodiimide (WSCD) hydrochloride (1.41 g) in an ice-water bath under nitrogen gas atmosphere. After 30 minutes, the mixture was stirred at ambient temperature. After stirring for 8 hours, the reaction mixture was washed with water and brine, dried over magnesium sulfate and evaporated in vacuo. The residue was purified by flash silica gel column chromatography (silica gel, 200 ml) eluting with hexane-ethyl acetate=5-1,4-1, 3-1, and 2-1 to give 2-(trimethylsilyl)ethyl [(2S)-2-(5-bromothiophen-2-yl)-1,1-dioxo-3,4,5,6-tetrahydro-2H-thiopyran-2-yl]acetate as colorless gum (2.66 g).

NMR (CDCl₃, δ): 0.01 (9H, s), 0.75-0.89 (2H, m), 1.72-2.24 (4H, m), 2.62-2.81 (2H, m), 2.99-3.18 (3H, m), 3.34 (1H, d, J=15 Hz), 3.95-4.09 (2H, m), 6.96-7.06 (2H, m)

Preparation 2-1)

A mixture of 4-bromophenylboronic acid (600 mg) and 4-bromobenzonitrile (1.63 g) in dioxane (12 ml) was added dichlorobis(triphenylphosphine)palladium(II) (63 mg) and 2M sodium carbonate (12 ml) at ambient temperature. The mixture was heated at 80° C. for 12 hours. The cooled reaction mixture was concentrated to about ¼ volume. To this were added chloroform and water. The organic layer was separated, washed with brine, dried over magnesium sulfate, and evaporated in vacuo. The residue was purified by flash silica gel chromatography (silica gel, 100 ml) eluting with hexane-chloroform=5-1 and followed by trituration with isopropyl ether to give 4-bromo-4′-cyanobiphenyl as a colorless solid (452 mg).

NMR (CDCl₃, δ): 7.46 (2H, d, J=8 Hz), 7.56-7.70 (4H, m), 7.73 (2H, d, J=8 Hz)

Preparation 2-2)

To a suspension of potassium acetate (175 mg) in dioxane (1 ml) were added 4-bromo-4′-cyanobiphenyl (115 mg), bis(pinacolato)diborane (113 mg) and dichlorobis(triphenylphosphine)palladium(II) (9 mg) at ambient temperature under nitrogen atmosphere. The mixture was heated at 80° C. for 16 hours to give crude 4′-cyano-4-biphenylboronic acid pinacol cyclic ester, which was used to the next reaction without purification.

Preparation 3-1)

4-Bromo-4′-ethoxybiphenyl (800 mg) was obtained from 4-ethoxyphenylboronic acid in a similar manner to that of

Preparation 2-1).

NMR (DMSO-d₆, δ): 1.35 (3H, t, J=8 Hz), 4.06 (2H, q, J=8 Hz), 7.00 (2H, d, J=8 Hz), 7.54-7.64 (6H, m)

Preparation 3-2)

4′-Ethoxy-4-biphenylboronic acid pinacol cyclic ester was obtained from 4-bromo-4′-ethoxybiphenyl (123 mg) in a similar manner to that of Preparation 2-2).

The following compounds were obtained in a similar manner to that of Preparation 2-2).

Preparation 4-1)

Benzofuran-5-boronic acid pinacol cyclic ester

Preparation 4-2)

Naphthalene-2-boronic acid pinacol cyclic

Preparation 4-3)

4-(2-Oxazolyl)benzeneboronic acid pinacol cyclic ester

Preparation 4-4)

4-(5-Oxazolyl)benzeneboronic acid pinacol cyclic ester

Preparation 5

4-(2-Oxazolyl)benzeneboronic acid pinacol cyclic ester

Preparation 6

2,3-Dihydrobenzofuran-5-boronic acid pinacol cyclic ester

Preparation 7-1)

To a solution of 4-bromophenol (5.09 g) and potassium carbonate (6.10 g) in dimethylformamide (25 ml) was added benzyl bromide (5.18 g) under ice-water cooling and the mixture was stirred at ambient temperature for 1 hour, and at 60° C. for 1 hour. The mixture was partitioned between ethyl acetate and water. The organic layer was separated, washed with 1N hydrochloric acid, water, saturated sodium bicarbonate solution and brine, dried over magnesium sulfate, and evaporated in vacuo to give 1-benzyloxy-4-bromobenzene as a colorless amorphous powder (7.0 g).

NMR (CDCl₃, δ): 5.03 (2H, s), 6.84 (2H, d, J=8 Hz), 7.29-7.46 (7H, m)

Preparation 7-2)

To a solution of 1-benzyloxy-4-bromobenzene (5.00 g) in tetrahydrofuran (50 ml) was added 1.5N n-butyllithium-hexane solution (14 ml) under dry ice-acetone cooling and the mixture was stirred at −60° C. for 0.5 hour. Tri-1-propyl borate (4.65 g) was added at −60° C. and the mixture was stirred at ambient temperature for 2 hours. 0.3N Hydrochloric acid (50 ml) was added and the mixture was stirred at ambient temperature for 1 hour, extracted with ethyl acetate. The organic layer was separated, washed with brine, dried over magnesium sulfate, and evaporated in vacuo. The residue was triturated with hexane to give 4-benzyloxybenzeneboronic acid as a colorless amorphous powder (4.12 g).

NMR (DMSO-d₆, δ): 5.12 (2H, s), 6.96 (2H, d, J=8 Hz), 7.28-7.50 (5H, m), 7.73 (2H, d, J=8 Hz), 7.86 (2H, s)

Preparation 8

Quinoline-3-boronic acid pinacol cyclic ester was obtained in a similar manner to that of Preparation 2-2).

Preparation 9

Quinoline-6-boronic acid pinacol cyclic ester was obtained in a similar manner to that of Preparation 2-2).

Preparation 10

To a solution of [(2S)-2-(5-bromothiophen-2-yl)-1,1-dioxo-3,4,5,6-tetrahydro-2H-thiopyran-2-yl]acetic acid (5.0 g) in tetrahydrofuran (50 ml) was added N,N-dimethylformamide di-tert-butyl acetal (11.5 g) at room temperature. After being stirred at 50° C. overnight, the mixture was concentrated in vacuo. The residue was dissolved in AcOEt (ethyl acetate)(100 ml) and the solution was washed with 1M NaHCO₃ (sodium hydrogen carbonate) aqueous solution and brine, dried over MgSO₄ (magnesium sulfate), and concentrated in vacuo. The resulting residue was purified by SiO₂ (silica gel) column chromatography (Hexane/AcOEt, 4:1) to give tert-butyl [(2S)-2-(5-bromothiophen-2-yl)-1,1-dioxo-3,4,5,6-tetrahydro-2H-thiopyran-2-yl]acetate (2.66 g) as an oil.

This product was immediately used for the next step.

The following compounds were obtained in a similar manner to that of Preparation 2-2). These products were immediately used for the next reaction.

Preparation 11

4-(2-Thienyl)phenylboronic acid pinacol ester

Preparation 12

Benzothiophene-5-boronic acid pinacol ester

Preparation 13

6-Methoxycarbonylnaphthalene-2-boronic acid pinacol ester

Preparation 14

6-Methoxynaphthalene-2-boronic acid pinacol ester

Preparation 15

6-Formylnaphthalene-2-boronic acid pinacol ester

Preparation 16-1)

A mixture of 2-bromo-6-naphthylaldehyde (1.41 g), p-toluenesulfonylmethylisocyanide (1.21 g), and potassium carbonate (1.24 g) in methanol(14 ml) was refluxed for 3 hours (under nitrogen atmosphere). After cooling, methanol was evaporated off. The residue was diluted with water and extracted with ethyl acetate. The organic layer was washed with saturated aqueous ammonium chloride, water, brine, dried over anhydrous magnesium sulfate and concentrated in vacuo. The residue was purified by flash column chromatography on silica gel eluting with a mixture of ethyl acetate and n-hexane (1:4) to give 2-bromo-6-(oxazol-5-yl)naphthalene (670 mg) as a colorless crystal.

NMR (CDCl₃, δ): 7.48 (1H, s), 7.59 (1H, dd, J=8, 1 Hz), 7.73-7.84 (3H, m), 8.00 (1H, d, J=8 Hz), 8.11 (1H, s)

MS (m/z): 274 (M+), 276 (M⁺+2), 115 (bp)

The following compounds were obtained in a similar manner to that of Preparation 2-2). These products were immediately used for the next reaction.

Preparation 16-2)

6-(Oxazol-5-yl)naphthalene-2-boronic acid pinacol ester

Preparation 17

6-Hydroxynaphthalene-2-boronic acid pinacol ester

Preparation 18

Benzothiophene-3-boronic acid pinacol ester

Preparation 19

6-Cyanonaphthalene-2-boronic acid pinacol ester

Preparation 20

6-Ethoxynaphthalene-2-boronic acid pinacol ester

Preparation 21

1-Methyl-2,3-dihydro-2-oxoindole-5-boronic acid pinacol ester

Preparation 22

6-Methoxymethoxynaphthalene-2-boronic acid pinacol ester

Preparation 23

To a stirred solution of 5-fluorobenzothiophene (1 g) in tetrahydrofuran (10 ml) was added n-butyl lithium (1.59M, 5.8 ml) at −78° C. and the reaction mixture was stirred at the same temperature for 1 hour and 0° C. for 30 minutes. Triisopropylborate (2.12 ml) was added at −60° C., and the reaction mixture was stirred at −60° C. for 1 hour and at room temperature for 4 hours. The reaction mixture was quenched with 1N aqueous hydrochloric acid and extracted with ethyl acetate. The organic layer was washed with 1N aqueous sodium hydroxide. The aqueous layer was acidified with 12N hydrochloric acid and extracted with ethyl acetate. The organic layer was washed with brine, dried over anhydrous magnesium sulfate and concentrated in vacuo. The solid was washed with ethyl acetate and n-hexane to give 5-fluorobenzothiophene-2-boronic acid (950 mg) as a brown crystal.

NMR (CDCl₃, δ): 4.74 (2H, s), 7.15 (1H, ddd, J=7.5, 7.5, 2 Hz), 7.25-7.29 (1H, m), 7.50 (1H, dd, J=7.5, 4 Hz)

Preparation 24

6-(Methylaminocarbonyl)naphthalene-2-boronic acid pinacol ester was obtained in a similar manner to that of Preparation 2-2).

Preparation 25

A mixture of methyl bromobenzoate (1 g), phenylenebisboronic acid (2.34 g), tetrakis(triphenylphosphine)palladium (107 mg), and 2M aqueous sodium carbonate (15 ml) in 1,4-dioxane (15 ml) was stirred at 80° C. for 3 hours. After cooling, water (50 ml) was added to this mixture and filtrated. The residue was washed with saturated aqueous sodium hydrogencarbonate. The filtrate was acidified to pH 2 by hydrochloric acid. The precipitate was collected to give 4-(4-methoxycarbonylphenyl)phenylboronic acid (672 mg) as a solid.

NMR (DMSO-d₆, δ): 3.34 (3H, s), 7.73 (4H, s), 8.02 (4H, s)

Preparation 26-11)

N-Methyl-4-(4-bromobenzene)benzenesulfonamide was obtained in a similar manner to that of Preparation 25.

NMR (CDCl₃, δ): 2.71 (3H, d, J=7 Hz), 4.34 (1H, q; J=7 Hz), 7.44-7.53 (2H, m), 7.58-7.80 (4H, m), 7.90-7.97 (2H, m)

Preparation 26-2)

4-(4-Methylaminosulfonylphenyl)benzene boronic acid pinacol ester was obtained in a similar manner to that of Preparation 2-2).

Preparation 27

4-(Pyrazin-2-yl)phenylboronic acid was obtained in a similar manner to that of Preparation 25.

NMR (DMSO-d₆, δ): 7.98 (2H, d, J=8 Hz), 8.11 (2H, d, J=8 Hz), 8.21 (2H, s), 8.62 (1H, d, J=2 Hz), 8.73 (1H, d, J=2 Hz), 9.28 (1H, s)

Preparation 28-1)

5-(4-Bromophenyl)pyrimidine was obtained in a similar manner to that of Preparation 37-1).

NMR (CDCl₃, 5): 7.46 (2H, d, J=8 Hz), 7.67 (2H, d, J=8 Hz), 8.93 (2H, s), 9.23 (1H, s)

Preparation 28-2)

4-(Pyrimidin-5-yl)phenylboronic acid pinacol ester was obtained in a similar manner to that of Preparation 2-2).

Preparation 29-1)

To a solution of 4′-bromophenacyl bromide (3.00 g) in ethanol was added thioacetamide (973 mg) and the mixture was heated under reflux for 4 hours. After evaporation of solvent, the residue was partitioned between ethyl acetate and saturated sodium bicarbonate solution. The organic layer was separated, washed with water and brine, dried over magnesium sulfate, and evaporated in vacuo. The residue was purified by silica gel column chromatography eluting with a mixture of chloroform and hexane (1:2-1:1) to give 4-(4-bromophenyl)-2-methylthiazole as a colorless powder (2.44 g).

NMR (CDCl₃, δ): 2.77 (3H, s), 7.31 (1H, s), 7.53 (2H, d, J=8 Hz), 7.76 (2H, d, J=8 Hz)

MS (m/z): 254, 256 (M+H)

Preparation 29-2)

4-(2-Methylthiazol-4-yl)phenylboronic acid pinacol ester was obtained in a similar manner to that of Preparation 2-2).

Preparation 30-1)

A solution of 4′-bromoacetophenone (2.00 g) in dimethylformamide dimethylacetal (60 ml) was stirred at 140° C. for 8 hours. After evaporation of solvent, the residue was purified by silica gel column chromatography eluting with a mixture of chloroform and methanol (100:1-20:1) to give 1-(4-bromophenyl)-3-(dimethylamino)prop-2-ene-1-one as a orange powder (2.30 g).

NMR (CDCl₃, δ): 2.87-3.05 (3H, br), 3.05-3.27 (3H, br), 5.66 (1H, d, J=15 Hz), 7.54 (2H, d, J=8 Hz), 7.79 (2H, d, J=8 Hz), 7.81 (1H, d, J=15 Hz)

MS (m/z): 254, 256 (M−H)

Preparation 30-2)

To a solution of 1-(4-bromophenyl)-3-(dimethylamino)prop-2-ene-1-one (2.43 g) in methanol (20 ml) was added hydroxylamine hydrochloride (997 mg) and the mixture was stirred at ambient temperature for 2 hours. After evaporation of solvent, the residue was partitioned between ethyl acetate and saturated sodium bicarbonate solution. The organic layer was separated, washed with brine, dried over magnesium sulfate, and evaporated in vacuo. The residue was purified by silica gel column chromatography eluting with a mixture of hexane and ethyl acetate (5:1) to give 5-(4-bromophenyl)isoxazole as a pale yellow powder (1.80 g).

NMR (CDCl₃, δ): 6.53 (1H, d, J=2 Hz), 7.61 (2H, d, J=8 Hz), 7.67 (2H, d, J=8 Hz), 8.30 (1H, d, J=2 Hz)

Preparation 31

4-(Isoxazol-5-yl)phenylboronic acid pinacol ester was obtained in a similar manner to that of Preparation 2-2).

Preparation 32-1)

To a suspension of thallium acetate(III) (1.99 g) in acetonitrile (160 ml) was added trifluoromethanesulfonic acid (3.39 g) and the mixture was stirred at ambient temperature for 10 minutes. 4′-Bromoacetophenone (1.00 g) was added and the mixture was heated under reflux for 1.5 hours. After evaporation of solvent, the residue was partitioned between chloroform and water. The organic layer was separated, washed with saturated sodium bicarbonate solution and brine, dried over magnesium sulfate, and evaporated in vacuo. The residue was purified by silica gel column chromatography eluting with a mixture of hexane and ethyl acetate (5:1-3:1). The crude product was triturated with hexane to give 5-(4-bromophenyl)-2-methyloxazole as a colorless powder (953 mg).

NMR (CDCl₃, δ): 2.54 (3H, s), 7.22 (1H, s), 7.48 (2H, d, J=8 Hz), 7.54 (2H, d, J=8 Hz)

Preparation 32-2)

4-(2-Methyloxazol-5-yl)phenylboronic acid pinacol ester was obtained in a similar manner to that of Preparation 2-2).

Preparation 33-1)

To a solution of 1-(4-bromophenyl)-3-(dimethylamino)prop-2-ene-1-one (2.46 g) in ethanol (25 ml) was added methylhydrazine (892 mg) and the mixture was stirred at ambient temperature for 8 hours. The mixture was partitioned between ethyl acetate and brine. The organic layer was separated, dried over magnesium sulfate, and evaporated in vacuo. The residue was purified by silica gel column chromatography eluting with a mixture of hexane and ethyl acetate (5:1-3:1). The crude product was triturated with hexane to give 5-(4-bromophenyl)-1-methylpyrazole as a yellow powder (1.00 g).

NMR (CDCl₃, δ): 3.88 (3H, s), 6.30 (1H, d, J=2 Hz), 7.29 (2H, d, J=8 Hz), 7.49 (1H, d, J=2 Hz), 7.59 (2H, d, J=8 Hz)

Preparation 33-2)

4-(1-Methylpyrazol-5-yl)phenylboronic acid pinacol ester was obtained in a similar manner to that of Preparation 2-2).

Preparation 34-1)

To a solution of 4-bromobenzaldehyde (1.80 g) and p-toluenesulfonylmethylisocyanide (2.10 g) in methanol (20 ml) was added potassium carbonate (2.02 g) and the mixture was heated under reflux for 5 hours. After evaporation of solvent, the residue was partitioned between ethyl acetate and water. The organic layer was separated, washed with 1N hydrochloric acid, water, saturated sodium bicarbonate solution and brine, dried over magnesium sulfate, and evaporated in vacuo. The residue was purified by silica gel column chromatography eluting with a mixture of hexane and ethyl acetate (5:1-3:1). The crude product was triturated with hexane to give 5-(4-bromophenyl)-4-methyloxazole as a pale brown powder (1.64 g).

NMR (CDCl₃, δ): 2.44 (3H, s), 7.48 (2H, d, J=8 Hz), 7.59 (2H, d, J=8 Hz), 7.85 (1H, s)

MS (m/z): 238, 240 (M+H)

Preparation 34-2)

4-(0.4-Methyloxazol-5-yl)phenylboronic acid pinacol ester was obtained in a similar manner to that of Preparation 2-2).

Preparation 35-1)

To a solution of 4-bromo-2-methylbenzoic acid (4.00 g) in tetrahydrofuran (40 ml) was added dropwise 2M borane dimethylsulfide complex solution (18.6 ml) and the mixture was heated under reflux for 2 hours. 1N Hydrochloric acid was added and the mixture was heated under reflux for 0.5 hour, and extracted with ethyl acetate. The organic layer was separated, washed with water, saturated sodium bicarbonate solution and brine, dried over magnesium sulfate, and evaporated in vacuo to give 4-bromo-2-methylbenzyl alcohol as a yellow oil (4.02 g).

NMR (CDCl₃, δ): 1.63 (1H, br), 2.32 (3H, s), 4.63 (2H, s), 7.18-7.37 (3H, m)

Preparation 35-2)

To a solution of oxalyl chloride (2.09 ml) in dichloromethane (80 ml) was added dropwise dimethylsulfoxide (3.69 ml) under dryice-acetone cooling and the mixture was stirred at −60° C. for 15 minutes. To the solution was added dropwise 4-bromo-2-methylbenzyl alcohol (4.02 g) and the mixture was stirred at −60° C. for 1 hour. Triethylamine (9.75 ml) was added portionwise and the mixture was stirred at ambient temperature for 2 hours. The solution was washed with water, 1N hydrochloric acid, water, saturated sodium bicarbonate solution and brine, dried over magnesium sulfate, and evaporated in vacuo. The residue was purified by silica gel column chromatography eluting with a mixture of hexane and ethyl acetate (20:1-10:1) to give 4-bromo-2-methylbenzaldehyde as a colorless oil (3.66 g).

NMR (CDCl₃, δ): 2.66 (3H, s), 7.46 (1H, s), 7.52 (1H, d, J=8 Hz), 7.67 (1H, d, J=8 Hz), 10.22 (1H, br)

Preparation 36-1)

To a solution of 4-bromo-2-methylbenzaldehyde (3.46 g) and p-toluenesulfonylmethylisocyanide (3.50 g) in methanol (50 ml) was added potassium carbonate (3.60 g) and the mixture was heated under reflux for 2 hours. After evaporation of solvent, the residue was partitioned between ethyl acetate and water. The organic layer was separated, washed with 1N hydrochloric acid, water, saturated sodium bicarbonate solution and brine, dried over magnesium sulfate, and evaporated in vacuo. The residue was purified by silica gel column chromatography eluting with a mixture of hexane and ethyl acetate (5:1-3:1) to give 5-(4-bromo-2-methylphenyl)oxazole as a pale yellow powder (3.48 g).

NMR (CDCl₃, δ): 2.46 (3H, s), 7.26 (1H, m), 7.36-7.45 (2H, m), 7.55 (1H, d, J=8 Hz), 7.97 (1H, s)

Preparation 36-2)

3-Methyl-4-(oxazol-5-yl)phenylboronic acid pinacol ester was obtained in a similar manner to that of Preparation 2-2).

Preparation 37-1)

To a solution of 4-chlorophenylboronic acid (5.00 g), 1,4-dibromobenzene (15.1 g) and dichlorobis(triphenylphosphine)palladium(II) (PdCl₂PPh₃)₂ (673 mg) in 1,4-dioxane was added 2M sodium bicarbonate solution (64 ml) and the mixture was stirred at 80° C. for 3 hours. After evaporation of solvent, ethyl acetate and water were added, and the insolubles were filtered off. The organic layer was separated, washed with brine, dried over magnesium sulfate, and evaporated in vacuo. The residue was purified by silica gel column chromatography eluting with hexane to give 4-bromo-4′-chlorobiphenyl as a colorless powder (3.12 g).

NMR (CDCl₃, δ): 7.36-7.52 (6H, m), 7.57 (2H, d, J=8 Hz)

Preparation 37-2)

To a solution of 4-bromo-4′-chlorobiphenyl (6.61 g) in tetrahydrofuran (100 ml) was added dropwise 1.5M n-butyllithium hexane solution (18 ml) under dryice-acetone cooling and the mixture was stirred at −60° C. for 0.5 hour. To the mixture was added dropwise tri-1-propyl borate (7.41 ml) and the mixture was stirred at ambient temperature for 3 hours. 0.5N Hydrochloric acid was added, and the mixture was stirred at ambient temperature for 0.5 hour and extracted with ethyl acetate. The organic layer was separated, washed with brine, dried over magnesium sulfate, and evaporated in vacuo. The residue was triturated with hexane to give 4-(4-chlorophenyl)phenylboronic acid as a colorless powder (4.00 g).

NMR (DMSO-d₆, δ): 7.52 (2H, d, J=8 Hz), 7.63 (2H, d, J=8 Hz), 7.71 (2H, d, J=8 Hz), 7.88 (2H, d, J=8 Hz), 8.11 (2H, s)

Preparation 38-1)

4-Bromo-4′-ethylbiphenyl was obtained in a similar manner to that of Preparation 37-1).

NMR (CDCl₃, δ): 1.29 (3H, t, J=7 Hz), 2.70 (2H, q, J=7 Hz), 7.28 (2H, m), 7.35-7.57 (6H, m)

Preparation 38-2)

4-(4-Ethylphenyl)phenylboronic acid was obtained in a similar manner to that of Preparation 37-2).

NMR (DMSO-d₆, δ): 1.21 (3H, t, J=7 Hz), 2.64 (2H, q, J=7 Hz), 7.30 (2H, d, J=8 Hz), 7.60 (4H, d, J=8 Hz), 7.86 (2H, d, J=8 Hz), 8.07 (2H, s)

Preparation 39-1)

4-Bromo-4′-trifluoromethylbiphenyl was obtained in a similar manner to that of Preparation 37-1).

NMR (CDCl₃, δ): 7.45 (2H, d, J=8 Hz), 7.53-7.74 (6H, m)

Preparation 39-2)

4-(4-Trifluoromethylphenyl)phenylboronic acid was obtained in a similar manner to that of Preparation 37-2).

NMR (DMSO-d₆, δ): 7.72 (2H, d, J=8 Hz), 7.81 (2H, d, J=BHz), 7.92 (4H, m), 8.16 (2H, s)

Preparation 40-1)

4-Bromo-4′-methylthiobiphenyl was obtained in a similar manner to that of Preparation 37-1.

NMR (CDCl₃, δ): 2.52 (3H, s), 7.32 (2H, d, J=8 Hz), 7.38-7.57 (6H, m)

Preparation 40-2)

4-(4-Methylthiophenyl)phenylboronic acid was obtained in a similar manner to that of Preparation 37-2).

NMR (DMSO-d₆, δ): 2.51 (3H, s), 7.35 (2H, d, J=8 Hz), 7.55-7.70 (4H, m), 7.86 (2H, d, J=8 Hz), 8.08 (2H, s)

Preparation 41

4-(4-Acetylphenyl)phenylboronic acid pinacol ester was obtained in a similar manner to that of Preparation 2-2).

Preparation 42

4-(4-t-Butylphenyl)phenylboronic acid was obtained in a similar manner to that of Preparation 37-2).

NMR (DMSO-d₆, δ): 1.32 (9H, s), 7.48 (2H, d, J=8 Hz), 7.61 (4H, d, J=8 Hz), 7.86 (2H, d, J=8 Hz), 8.07 (2H, s)

Preparation 43-1)

4-Bromo-4′-fluorobiphenyl was obtained in a similar manner to that of Preparation 37-1).

NMR (CDCl₃, δ): 7.06-7.16 (2H, m), 7.40 (2H, d, J=8 Hz), 7.45-7.58 (4H, m)

Preparation 43-2)

4-(4-Fluorophenyl)phenylboronic acid was obtained in a similar manner to that of Preparation 37-2).

NMR (DMSO₆, δ): 7.20-7.33 (2H, m), 7.55-7.75 (4H, m), 7.80-7.95 (2H, m), 8.08 (2H, br)

Preparation 44-1)

4-Bromo-4′-methoxybiphenyl was obtained in a similar manner to that of Preparation 37-1.

NMR (CDCl₃, δ): 3.86 (3H, s), 6.98 (2H, d, J=8 Hz), 7.38 (2H, d, J=8 Hz), 7.43-7.54 (4H, m)

Preparation 44-2)

4-(4-Methoxyphenyl)phenylboronic acid was obtained in a similar manner to that of Preparation 37-2).

NMR (DMSO-d₆, δ): 3.80 (3H, s), 7.02 (2H, d, J=8 Hz), 7.56-7.67 (4H, m), 7.84 (2H, d, J=8 Hz), 8.07 (2H, br)

Preparation-45

4-(4-Hydroxyphenyl)phenylboronic acid pinacol ester was obtained in a similar manner to that of Preparation 2-2).

Preparation 46-1)

4-Bromo-4′-trifluoromethoxybiphenyl was obtained in a similar manner to that of Preparation 37-1).

NMR (CDCl₃, δ): 7.22-7.30 (2H, m), 7.36 (2H, d, J=8 Hz), 7.47-7.57 (4H, m)

Preparation 46-2)

4-(4-Trifluoromethoxyphenyl)phenylboronic acid was obtained in a similar manner to that of Preparation 37-2).

NMR (DMSO-d₆, δ): 7.46 (2H, d, J=8 Hz), 7.65 (2H, d, J=8 Hz), 7.82 (2H, d, J=8 Hz), 7.89 (2H, d, J=8 Hz), 8.12 (2H, s)

Preparation 47-1)

To a suspension of calcium hypochlorite (3.48 g) in water (14 ml) was added a mixture of potassium carbonate (2.52 g) and potassium hydroxide (838 mg) in water (7 ml) and the insolubles were filtered off. To the filtrates were added 4,-(4-bromophenyl)acetophenone (2.00 g) in 1,4-dioxane (20 ml) under ice-water cooling and the mixture was stirred at 0° C. for 3 hours. After sodium sulfite (4.3 g) was added, the solution was acidified with 50% sulfuric acid. The resulting powder was collected and washed with water, methanol and ether to give 4-(4-bromophenyl)benzoic acid as a colorless powder (1.93 g).

NMR (DMSO-d₆, δ): 7.70 (4H, s), 7.81 (2H, d, J=8 Hz), 8.02 (2H, d, J=8 Hz), 13.04 (1H, br)

MS (m/z): 275, 277 (M−H)

Preparation 47-2)

To a solution of 4-(4-bromophenyl)benzoic acid (600 mg), 1-ethyl-3-(31-dimethylaminopropyl)carbodiimide (504 mg) and 1-hydroxybenotriazole (439 mg) in dimethylformamide (6 ml) was added methylamine hydrochloride (175 mg) under ice-water cooling and the mixture was stirred at 0° C. for 2 hours. To the mixture was added water and the resulting precipitates were collected, washed with water to give N-methyl-4-(4-bromophenyl)benzamide as a colorless powder (543 mg).

NMR (DMSO-d₆, δ): 2.80 (3H, d, J=7 Hz), 7.66-7.74 (4H, m), 7.77 (2H, d, J=8 Hz), 7.93 (2H, d, J=8 Hz), 8.51 (1H, br)

Preparation 48

4-(4-Methylcarbamoylphenyl)phenylboronic acid pinacol ester was obtained in a similar manner to that of Preparation 2-2).

Preparation 49-1)

N,N-Dimethyl-4-(4-bromophenyl)benzamide was obtained in a similar manner to that of Preparation 47-2).

NMR (DMSO-d₆, δ): 2.95 (3H, s), 3.00 (3H, s), 7.47 (2H, d, J=8 Hz), 7.68 (4H, s), 7.73 (2H, d, J=8 Hz)

Preparation 49-2)

4-(4-Dimethylcarbamoylphenyl)phenylboronic acid pinacol ester was obtained in a similar manner to that of Preparation 2-2).

Preparation 50-1)

4-(4-Bromophenyl)benzamide was obtained in a similar manner to that of Preparation 47-2).

NMR (DMSO-d₆, δ): 7.41 (1H, br), 7.64-7.72 (4H, m), 7.76 (2H, d, J=8 Hz), 7.97 (2H, d, J=8 Hz), 8.05 (1H, br)

Preparation 50-2)

4-(4-Carbamoylphenyl)phenylboronic acid pinacol ester was obtained in a similar manner to that of Preparation 2-2).

Preparation 51-1)

To a solution of 4-(4-bromophenyl)benzoic acid (600 mg) in dimethylformamide (10 ml) was added N,N′-carbonyldiimidazole (456 mg) and the mixture was stirred at ambient temperature for 1 hour. To the mixture were added methanesulfonamide (309 mg) and 1,8-diazabicyclo[5.4.0]-undec-7-ene (494 mg) and the mixture was stirred at ambient temperature for 3 hours. The mixture was partitioned between ethyl acetate and 1N hydrochloric acid. The organic layer was separated, washed with water and brine, dried over magnesium sulfate, and evaporated in vacuo. The residue was triturated with ether to give N-(4-(4-bromophenyl)benzoyl)methanesulfonamide as a colorless powder (692 mg).

NMR (DMSO-d₆, δ): 3.39 (3H, s), 7.66-7.79 (4H, m), 7.85 (2H, d, J=BHz), 8.04 (2H, d, J=8 Hz), 12.22 (1H, br).

MS (m/z): 352, 354 (M−H)

Preparation 51-2)

4-(4-Methylsulfonylaminocarbonylphenyl)phenylboronic acid pinacol ester was obtained in a similar manner to that of Preparation 2-2).

Preparation 52-1)

4-(4-Bromophenyl)benzyl alcohol was obtained in a similar manner to that of Preparation 35-1).

NMR (CDCl₃, δ): 1.69 (1H, t, J=7 Hz), 4.76 (2H, d, J=7 Hz), 7.45 (4H, m), 7.55 (4H, m)

Preparation 52-2)

4-(4-Hydroxymethylphenyl)phenylboronic acid pinacol ester was obtained in a similar manner to that of Preparation 2-2).

Preparation 53-1)

4-(4-Bromophenyl)benzaldehyde was obtained in a similar manner to that of Preparation 35-2).

NMR (CDCl₃, δ): 7.50 (2H, d, J=8 Hz), 7.62 (2H, d, J=8 Hz), 7.72 (2H, d, J=8 Hz), 7.96 (2H, d, J=8 Hz), 10.06 (1H, s)

Preparation 53-2)

5-(4-(4-Bromophenyl)phenyl)oxazole was obtained in a similar manner to that of Preparation 36-1).

NMR (CDCl₃, δ): 7.39 (1H, s), 7.48 (2H, d, J=8 Hz), 7.54-7.66 (4H, m), 7.73 (2H, d, J=8 Hz), 7.95 (1H, s)

Preparation 54

4-(4-(Oxazol-5-yl)phenyl)phenylboronic acid pinacol ester was obtained in a similar manner to that of Preparation 2-2).

Preparation 55-1)

To a solution of 4-bromophenylbenzoic acid (1.30 g) in t-butyl alcohol (20 ml) was added triethylamine (0.937 ml) and diphenylphosphoryl azide (1.45 ml) and the mixture was heated under reflux for 1 hour. After evaporation of solvent, ethyl acetate and water were added. The insolubles were filtered off, and the organic layer of the filtrates was separated, washed with 1N hydrochloric acid, water, saturated sodium bicarbonate solution and brine, and evaporated in vacuo. The residue was purified by silica gel column chromatography eluting with a mixture of chloroform and hexane (1:2) to give 4-bromo-4′-t-butoxycarbonylaminobiphenyl as a colorless powder (1.35 g).

NMR (DMSO-d₆, δ): 1.49 (9H, s), 7.52-7.65 (8H, m), 9.48 (1H, s)

Preparation 55-2)

4-(4-t-Butoxycarbonylaminophenyl)phenylboronic acid pinacol ester was obtained in a similar manner to that of Preparation 2-2).

Preparation 56-1)

4-Bromo-4′-methylbiphenyl was obtained in a similar manner to that of Preparation 37-1).

NMR (CDCl₃, δ): 2.40 (3H, s), 7.18-7.27 (2H, m), 7.37-7.48 (4H, m), 7.48-7.58 (2H, m)

Preparation 56-2)

4-(4-Methylphenyl)phenylboronic acid was obtained in a similar manner to that of Preparation 37-2).

NMR (DMSO-d₆, δ): 2.35 (3H, s), 7.28 (2H, d, J=8 Hz), 7.60 (4H, t, J=8 Hz), 7.86 (2H, d, J=8 Hz), 8.07 (2H, s)

Preparation 57-1)

4-Bromo-3′-methylbiphenyl was obtained in a similar manner to that of Preparation 37-1).

NMR (CDCl₃, δ): 2.42 (3H, s), 7.30-7.36 (4H, m), 7.43 (2H, d, J=8 Hz), 7.55 (2H, d, J=8 Hz)

Preparation 57-2)

4-(3-Methylphenyl)phenylboronic acid was obtained in a similar manner to that of Preparation 37-2).

NMR (DMSO-d₆, δ): 2.38 (3H, s), 7.19 (1H, d, J=8 Hz), 7.35 (1H, t, J=8 Hz), 7.45-7.53 (2H, m), 7.62 (2H, d, J=8 Hz), 7.87 (2H, d, J=8 Hz), 8.08 (2H, s)

Preparation 58-1)

4-Bromo-2′-methylbiphenyl was obtained in a similar manner to that of Preparation 37-1).

NMR (CDCl₃, δ): 2.25 (3H, s), 7.12-7.26 (6H, m), 7.53 (2H, d, J=8 Hz)

Preparation 58-2)

4-(2-Methylphenyl)phenylboronic acid was obtained in a similar manner to that of Preparation 37-2).

NMR (DMSO-d₆, δ): 2.23 (3H, s), 7.17-7.33 (6H, m), 7.84 (2H, d, J=8 Hz), 8.08 (2H, s)

Preparation 59

4-(2-Phenyl-2H-tetrazol-5-yl)phenylboronic acid pinacol ester was obtained in a similar manner to that of Preparation 2-2).

Preparation 60-1)

To a solution of ethyl (E)-4-bromocinnamate (5.00 g) in tetrahydrofuran (100 ml) was added dropwise 1M diisobutylaluminum hydride toluene solution (58.8 ml) at dryice—acetone cooling and the mixture was stirred at −78° C. for 1 hour. The mixture was partitioned between ethyl acetate and 1N hydrochloric acid. The organic layer was separated, washed with brine, dried over magnesium sulfate, and evaporated in vacuo. The residue was triturated with hexane to give (E)-4-bromocinnamyl alcohol as a colorless powder (3.89 g).

NMR (CDCl₃, δ): 1.48 (1H, br), 4.32 (2H, m), 6.81-6.92 (1H, dt, J=7 Hz, 15 Hz), 6.56 (1H, d, J=15 Hz), 7.26 (2H, d, J=8 Hz), 7.44 (2H, d, J=8 Hz)

Preparation 60-2)

(E)-4-Bromocinnamaldehyde was obtained in a similar manner to that of Preparation 35-2).

NMR (CDCl₃, δ): 6.66-6.76 (1H, dd, J=7 Hz, 15 Hz), 7.42 (1H, d, J=15 Hz), 7.43 (2H, d, J=8 Hz), 7.58 (2H, d, J=8 Hz), 9.71 (1H, d, J=7 Hz)

Preparation 60-3)

(E)-5-(2-(4-Bromophenyl)ethenyl)oxazole was obtained in a similar manner to that of Preparation 36-1).

NMR (CDCl₃, δ): 6.91 (1H, d, J=15 Hz), 7.04 (1H, d, J=15 Hz), 7.08 (1H, s), 7.34 (2H, d, J=8 Hz), 7.49 (2H, d, J=8 Hz), 7.86 (1H, s)

MS (m/z): 250, 252 (M+H)

Preparation 60-4)

(E)-4-(2-(Oxazol-5-yl)ethenyl)phenylboronic acid pinacol ester was obtained in a similar manner to that of Preparation 2-2).

Preparation 61-1)

4-Bromophenoxyacetaldehyde was obtained in a similar manner to that of Preparation 35-2).

NMR (CDCl₃, δ): 4.56 (2H, s), 6.76-6.86 (2H, m), 7.34-7.45 (2H, m), 9.83 (1H, s)

Preparation 61-2

5-(4-Bromophenoxymethyl)oxazole was obtained in a similar manner to that of Preparation 36-1).

NMR (CDCl₃, δ): 5.05 (2H, s), 6.85 (2H, d, J=8 Hz), 7.17 (1H, s), 7.41 (2H, d, J=8 Hz), 7.91 (1H, s)

Preparation 61-3)

4-(Oxazol-5-ylmethoxy) phenylboronic acid pinacol ester was obtained in a similar manner to that of Preparation 2-2).

Preparation 62-1)

To a solution of ethyl (E)-4-bromocinnamate (5.00 g) in ethanol (50 ml) was added 2M sodium hydroxide solution (15 ml) and the mixture was stirred at 60° C. for 1 hour. The solution was acidified with 1M hydrochloric acid and the resulting precipitates were collected, washed with water to give (E)-4-bromocinnamic acid as a colorless powder (4.30 g).

NMR (DMSO-d₆, δ): 6.58 (1H, d, J=15 Hz), 7.58 (1H, d, J=15 Hz), 7.58-7.69 (4H, m)

MS (m/z): 227 (M−H)

Preparation 62-2)

(E)-N-Methyl-4-bromocinnamamide was obtained in a similar manner to that of Preparation 47-2).

NMR (DMSO-d₆, δ): 2.70 (3H, d, J=7 Hz), 6.62 (1H, d, J=15 Hz), 7.38 (1H, d, J=15 Hz), 7.51 (2H, d, J=8 Hz), 7.60 (2H, d, J=8 Hz), 8.06 (1H, br)

Preparation 62-3)

(E)-4-(2-(Methylcarbamoyl) ethenyl) phenylboronic acid pinacol ester was obtained in a similar manner to that of Preparation 2-2).

Preparation 63-1)

To a solution of 4-bromoaniline (5.00 g) and triethylamine (6.08 ml) in dichloromethane (50 ml) was added acetoxyacetyl chloride (3.44 ml) and the mixture was stirred at ambient temperature for 2 hours. After evaporation of solvent, the residue was partitioned between ethyl acetate and 1N hydrochloric acid. The organic layer was separated, washed with water, saturated sodium bicarbonate solution and brine, dried over magnesium sulfate, and evaporated in vacuo. The residue was triturated with a mixture of hexane and ethyl acetate (5:1) to give 4′-bromo-2-acetoxyacetanilide as a pale brown powder (7.50 g).

NMR (CDCl₃, δ): 2.24 (3H, s), 4.69 (2H, s), 7.46 (4H, s), 7.78 (1H, br)

Preparation 63-2)

To a solution of 4′-bromo-2-acetoxyacetanilide (7.00 g) in methanol (50 ml) and tetrahydrofuran (30 ml) was added 2M sodium hydroxide solution (20 ml) and the mixture was stirred at ambient temperature for 2 hours. After evaporation of organic solvent, the aqueous layer was diluted with saturated sodium bicarbonate solution and extracted with ethyl acetate and tetrahydrofuran. The organic layer was separated, washed water and brine, dried over magnesium sulfate, and evaporated in vacuo. The residue was triturated with a mixture of hexane and ethyl acetate (5:1) to give 4′-bromo-2-hydroxyacetanilide as a colorless powder (5.64 g).

NMR (DMSO-d₆, δ): 3.98 (2H, s), 5.69 (1H, br), 7.49 (2H, d, J=8 Hz), 7.70 (2H, d, J=8 Hz)

MS (m/z): 228, 230 (M−H)

Preparation 63-3)

4′-Bromoglyoxylanilide was obtained in a similar manner to that of Preparation 35-2).

NMR (CDCl₃, δ): 7.30-7.56 (4H, m), 8.85 (1H, s)

MS (m/z): 226, 228 (M−H)

Preparation 63-4)

N-(4-Bromophenyl)oxazol-5-carboxamide was obtained in a similar manner to that of Preparation 36-1).

NMR (DMSO-d₆, δ): 7.57 (2H, d, J=8 Hz), 7.72 (2H, d, J=8 Hz), 8.00 (1H, s), 8.68 (1H, s), 10.58 (1H, br)

Preparation 63-5)

4-(Oxazol-5-ylcarbonylamino)phenylboronic acid pinacol ester was obtained in a similar manner to that of Preparation 2-2).

Preparation 64-1)

To a solution of 5-(4-bromophenyl)oxazole (2 g) and Et₃N (triethylamine) (6.3 ml) in AcOEt (10 ml) was added (trimethylsilyl)acetylene (2.63 g), PdCl₂(Ph₃P)₂ (125 mg) and CuI (copper Iodide) (68 mg), and the mixture was heated at 50° C. for 6 hours. After cooling, the reaction mixture was concentrated. The residue was purified by chromatography on silica gel (eluent: hexane−AcOEt=20-1) to give 5-{4-[(trimethylsilyl)ethynyl]phenyl)oxazole (1.27 g).

NMR (CDCl₃, δ): 0.26 (9H, s), 7.38 (1H, s), 7.52 (2H, d, J=8 Hz), 7.60 (2H, d, J=8 Hz), 7.92 (1H, s)

MS (ESI): m/z 242 (M+1)

Preparation 64-2)

A solution of 5-{4-[(trimethylsilyl)ethynyl]phenyl}-oxazole (1 g) in MeOH (methanol)(20 ml) was added 1M K₂CO₃aq. (aqueous potassium carbonate) (1.5 ml), and the mixture was stirred at room temperature for 6 hours. The reaction mixture was concentrated and extracted with AcOEt. The organic layer was washed with brine, dried over MgSO₄ and evaporated in vacuo. The residue was purified by chromatography on silica gel to give 5-(4-ethynylphenyl)oxazole (682 mg).

NMR (CDCl₃, δ): 3.17 (1H, s), 7.40 (1H, s), 7.55 (2H, d, J=8 Hz), 7.63 (2H, d, J=8 Hz), 7.94 (1H, s)

MS (ESI): m/z 170 (M+1)

Preparation 65-1)

A mixture of 2,4′-dibromoacetophenone (5.0 g) and formamide (5.67 g) was heated at 130° C. for 2 hours, then partitioned between ethyl acetate and water. The organic layer was separated, washed with water and brine, dried over magnesium sulfate, and evaporated. The residue was chromatographed on silica gel eluting with a mixture of ethyl acetate and hexane (1:5) to give 4-(4-bromophenyl)oxazole (1.37 g) as pale yellow crystals.

NMR (CDCl₃, δ): 7.54 (2H, dd, J=2, 7 Hz), 7.63 (2H, dd, J=2, 7 Hz), 7.94 (1H, s), 7.96 (1H, s)

Preparation 65-2)

A mixture of 4-(4-bromophenyl)oxazole (300 mg), bis(pinacolato)diboron (340 mg), tetrakis(triphenylphosphine)palladium(0) (46.4 mg), and potassium acetate (526 mg) in 1,4-dixane (6 ml) was heated at 80° C. for 2 hours to give the crude product of [4-(oxazol-4-yl)phenyl]boronic acid pinacol ester. The reaction mixture was used for the next step without isolation.

Preparation 66-1)

A mixture of 2,4′-dibromoacetophenone (5.0 g), phosphorus pentasulfide (8.0 g), and formamide (9.7 g) in 1,4-dioxane (20 ml) was heated 130° C. for 4 hours. The supernatant solution was separated and partitioned between ethyl acetate and water. The organic layer was separated, washed with water and brine, dried over magnesium sulfate, and evaporated in vacuo. The residue was chromatographed on silica gel eluting with a mixture of ethyl acetate and hexane (1:5) to give 4-(4-bromophenyl)thiazole (1.50 g) as pale yellow crystals.

NMR (CDCl₃, δ): 7.54 (2H, d, J=2 Hz), 7.56 (2H, d, J=9 Hz), 7.81 (2H, d, J=9 Hz), 8.88 (1H, s)

Preparation 66-2)

(4-(Thiazol-4-yl)phenyl]boronic acid pinacol ester was obtained in a similar manner to that of Preparation 2-2).

Preparation 67-1)

A suspension of polyphosphoric acid (8.6 g) in chlorobenzene (100 ml) was heated to reflux and a solution of 1-[(2,2-diethoxyethyl)thio]-3-fluorobenzene (7.0 g) in chlorobenzene (25 ml) was added over 2 hours. After the mixture was stirred for 1 hour with reflux, the supernatant solution was decanted and evaporated. The residue was partitioned between ether and 0.1N sodium hydroxide. The organic layer was separated, washed with water and brine, dried over magnesium sulfate, and evaporated. The residue was purified by silica gel column chromatograpy eluting with hexane to give 6-fluorobenzothiophene (1.79 g) as a yellow oil.

NMR (CDCl₃, δ): 7.13 (1H, dt, J=2, 9 Hz), 7.28 (1H, d, J=5 Hz), 7.38 (1H, d, J=5 Hz), 7.55 (1H, dd, J=2, 9 Hz), 7.75 (1H, dd, J=5, 9 Hz)

Preparation 67-2)

To a solution of 6-fluorobenzothiophene (705 mg) was added 1.6N butyl lithium in hexane (4.05 ml) at −65° C. and the mixture was stirred at the same temperature for 30 minutes, then at 40° C. for 30 minutes. The mixture was cooled to −65° C., then triisopropyl borate (1.22 g) was added. The mixture was allowed to warm up to 0° C. and stirred at 0° C. for 1 hour, then at 20° C. for 1 hour. After addition of 1N hydrochloric acid, the mixture was stirred at 20° C. for 30 minutes. The mixture was extracted with ethyl acetate, and the extract was washed with water and brine, dried over magnesium sulfate, and evaporated in vacuo. The residue was triturated with isopropyl alcohol to give (6-fluorobenzothiophen-2-yl)boronic acid (340 mg) as a yellow powder.

NMR (CDCl₃, δ): 7.25 (1H, dt, J=2, 9 Hz), 7.86 (1H, dd, J=2, 9 Hz), 7.92 (1H, dd, J=5, 9 Hz), 7.94 (1H, s), 8.51 (2H, s)

Preparation 68

To a solution of ((2S)-2-(5-bromothiophen-2-yl)-1,1-dioxo-3,4,5,6-tetrahydro-2H-thiopyran-2-yl)acetic acid (12.5 g), 4-methoxybenzyl alcohol (9.78 g) and 1-ethyl-3-(3′-dimethylaminopropyl)carbodiimide hydrochloride (10.2 g) in dichloromethane (200 ml) was added 4-dimethylaminopyridine (432 mg) and the mixture was stirred at ambient temperature for 15 hours. The mixture was partitioned between ethyl acetate and water. The organic layer was separated, washed with 1N hydrochloric acid, water, saturated sodium bicarbonate solution and brine, dried over magnesium sulfate, and evaporated in vacuo. The crude product was recrystallized with ethyl acetate to give 4-methoxybenzyl ((2S)-2-(5-bromothiophen-2-yl)-1,1-dioxo-3,4,5,6-tetrahydro-2H-thiopyran-2-yl)acetate as a colorless crystal (13.3 g).

NMR (CDCl₃, δ): 1.73-1.97 (2H, m), 2.03-2.24 (2H, m), 2.56-2.78 (2H, m), 2.98-3.16 (2H, m), 3.10 (1H, d, J=15 Hz), 3.36 (1H, d, J=15 Hz), 3.82 (3H, s), 4.86-4.97 (2H, m), 6.85 (2H, d, J=8 Hz), 6.93 (2H, m), 7.08 (2H, d, J=8 Hz)

Preparation 69

To a solution of tert-butyl [(2S)-2-(5-bromothiophen-2-yl)-1,1-dioxo-3,4,5,6-tetrahydro-2H thiopyran-2-yl]acetate (300 mg), 1,4-phenylenebisboronic acid (364 mg) in dioxane (10 ml) was added dichlorobis(triphenylphosphine)palladium(II) (15.4 mg) and 2M sodium carbonate (6 ml) at ambient temperature. After being stirred at 80° C. for 3 hours, the mixture was concentrated in vacuo. The residue was dissolved in AcOEt (10 ml) and the solution was washed with water, 0.5M HCl (hydrochloric acid) and brine, dried over MgSO4, and concentrated in vacuo. The obtained crude 4-{5-[(2S)-2-(tert-butoxycarbonylmethyl)-1,1-dioxo-3,4,5,6-tetrahydro-2H-thiopyran-2-yl]thiophen-2-yl)phenylboronic acid was used to the following reaction without purification.

### EXAMPLE 1

To 2-(trimethylsilyl)ethyl [(2S)-2-[5-(4′-cyano-4-biphenylyl)thiophen-s-yl]-1,1,-dioxo-1,1-dioxo-3,4,5,6-tetrahydro-2H-thiopyran-2-yl]acetate (110 mg) was added trifluoroacetic acid (0.3 ml) at ambient temperature. After 2-hour stirring, the reaction mixture was concentrated in vacuo to give a colorless solid (110 mg). The solid was triturated with ethyl acetate to give [(2S)-2-[5-(4′-cyano-4-biphenylyl)thiophen-2-yl]-1,1-dioxo-3,4,5,6-tetrahydro-2H-thiopyran-2-yl]acetic acid as a colorless solid (58 mg).

NMR (CDCl₃—CD₃OD, δ): 1.77-2.06 (2H, m), 2.08-2.25 (2H, m), 2.74-2.91 (2H, m), 3.09-3.25 (2H, m), 3.47 (1H, d, J=15.7 Hz), 7.27 (1H, d, J=4 Hz), 7.34 (1H, d, J=4 Hz), 7.60 (2H, d, J=8 Hz), 7.65-7.78 (6H, m)

The following compounds were obtained in a similar manner to that of Example 1.

### EXAMPLE 2

[(2S)-2-[5-(4′-Ethoxy-4-biphenylyl)thiophen-2-yl]-1,1-dioxo-3,4,5,6-tetrahydro-2H-thiopyran-2-yl]acetic acid (50 mg)

NMR (CDCl₃—CD₃OD, δ): 1.45 (3H, t, J=8 Hz), 1.77-2.05 (2H, m), 2.07-2.24 (2H, m), 2.78-2.87 (2H, m), 3.09-3.14 (3H, m), 3.46 (1H, d, J=15 Hz), 4.09 (2H, q, J=8 Hz), 6.96 (2H, d, J=BHz), 7.25 (1H, d, J=4 Hz), 7.29 (1H, d, J=4 Hz), 7.50-7.59 (4H, m), 7.63 (2H, d, J=8 Hz)

MS (ESI−): 469 (M−1)

### EXAMPLE 3

[(2S)-2-[5-[4-(5-Oxazolyl)phenyl]thiophen-2-yl]-1,1-dioxo-3,4,5,6-tetrahydro-2H-thiopyran-2-yl]acetic acid (53 mg)

NMR (CDCl₃—CD₃OD, 6): 1.77-2.04 (2H, m), 2.07-2.24 (2H, m), 2.71-2.89 (2H, m, overlapped with H₂O), 3.11-3.25 (3H, m), 3.46 (1H, d, J=15 Hz), 7.26 (1H, d, J=4 Hz), 7.31 (1H, d, J=4 Hz), 7.37 (1H, s), 7.65 (4H, s), 7.97 (1H, s)

MS (ESI+): 418 (M+1)

### EXAMPLE 4

[(2S)-2-[5-(4-n-Butylylphenyl)thiophen-2-yl]-1,1-dioxo-3,4,5,6-tetrahydro-2H-thiopyran-2-yl]acetic acid (18 mg)

NMR (CDCl₃, 5): 0.93 (3H, t, J=8 Hz), 1.27-1.44 (2H, m), 1.54-1.67 (2H, m), 1.70-2.24 (4H, m), 2.61 (2H, t, J=8 Hz), 2.65-2.90 (2H, m), 2.99-3.24 (3H, m), 3.47 (1H, d, J=15 Hz), 7.12-7.25 (4H, m), 7.49 (2H, d, J=8 Hz)

MS (ESI−): 405 (M−1)

### EXAMPLE 5

[(2S)-2-[5-[3-(3-n-Propylureido)phenyl]thiophen-2-yl]-1,1-dioxo-3,4,5,6-tetrahydro-2H-thiopyran-2-yl]acetic acid (27.5 mg)

NMR (CDCl₃, δ): 0.93 (2H, m), 1.53 (2H, m), 1.81-1.99 (2H, m), 2.16 (2H, m), 2.80 (2H, m), 3.14-3.20 (5H, m), 3.43 (1H, d, J=15 Hz), 7.18-7.24 (5H, m), 7.49 (1H, s)

MS (ESI+): 451.27 (MH)

MS (ESI−): 449.40 (M−H)

### EXAMPLE 6

[(2S)-2-[5-(4-Cyclohexylphenyl)thiophen-2-yl]-1,1-dioxo-3,4,5,6-tetrahydro-2H-thiopyran-2-yl]acetic acid (33.0 mg)

NMR (CDCl₃, δ): 1.22-1.49 (6H, m), 1.74-2.17 (10H, m), 2.51 (1H, m), 2.67-2.88 (2H, m), 3.01-3.20 (3H, m), 3.47 (1H, d, J=15 Hz), 7.18-7.23 (4H, m), 7.50 (2H, d, J=8 Hz)

MS (ESI−): 431.47 (M−H)

### EXAMPLE 7

[(2S)-2-[5-(2-Benzofuranyl)thiophen-2-yl]-1,1-dioxo-3,4,5,6-tetrahydro-2H-thiopyran-2-yl]acetic acid (23.0 mg)

NMR (CDCl₃, δ): 1.79-2.89 (6H, m), 3.03-3.23 (3H, m), 3.49 (1H, d, J=15 Hz), 6.88 (1H, s), 7.20-7.31 (3H, m), 7.41 (1H, d, J=4 Hz), 7.47 (1H, d, J=8 Hz), 7.55 (1H, d, J=7 Hz)

MS (ESI+): 399.49 (MH)

MS (ESI−): 779.16 (2M-H)

### EXAMPLE 8

[(2S)-2-[5-(4-n-Propyloxyphenyl)thiophen-2-yl]-1,1-dioxo-3,4,5,6-tetrahydro-2H-thiopyran-2-yl]acetic acid (4.8 mg)

NMR (CDCl₃, 5): 1.05 (3H, t, J=7 Hz), 1.76-2.15 (8H, m), 2.76 (2H, m), 3.00-3.18 (3H, m), 3.46 (3H, d, J=15 Hz), 3.94 (2H, t, J=7 Hz), 6.89 (2H, d, J=9 Hz), 7.00 (1H, d, J=5 Hz), 7.19 (1H, d, J=4 Hz), 7.49 (1H, d, J=9 Hz)

### EXAMPLE 9

[(2S)-2-[5-(4-Biphenylyl)thiophen-2-yl]-1,1-dioxo-3,4,5,6-tetrahydro-2H-thiopyran-2-yl]acetic acid (17.3 mg)

NMR (CDCl₃, δ): 1.79-2.19 (4H, m), 2.71-2.91 (2H, m), 3.04-3.23 (3H, m), 3.51 (1H, d, J=15 Hz), 7.29 (2H, m), 7.36 (1H, t, J=8 Hz), 7.45 (2H, t, J=8 Hz), 7.59-7.68 (6H, m)

MS (ESI−): 851.33 (2M-H)

### EXAMPLE 10

[(2S)-2-(5-(2-Naphthyl)thiophen-2-yl]-1,1-dioxo-3,4,5,6-tetrahydro-2H-thiopyran-2-yl]acetic acid (37.2 mg)

NMR (CDCl₃, 5): 1.80-2.18 (4H, m), 2.71-2.92 (2H, m), 3.05-3.23 (3H, m), 3.53 (1H, d, J=5 Hz), 7.29 (1H, d, J=4 Hz), 7.37 (1H, d, J=4 Hz), 7.44-7.52 (2H, m), 7.71 (1H, dd, J=2 and 9 Hz), 7.80-7.84 (3H, m), 8.05 (1H, s)

MS (ESI−): 799.19 (2M-H)

### EXAMPLE 11

To a crude mixture of 4-(2-oxazolyl)benzeneboronic acid pinacol cyclic ester obtained in Preparation 6-3) was added a solution of [(2S)-2-(5-bromothiophen-2-yl)-1,1-dioxo-3,4,5,6-tetrahydro-2H-thiopyran-2-yl]acetic acid (400 mg) in dioxane (10 ml), dichlorobis(triphenylphosphine)-palladium(II) (29.8 mg), and 2M sodium carbonate solution (2.84 ml) at room temperature. After the reaction mixture was heated at 80° C. for 3 hours, the solution was concentrated in vacuo to remove dioxane. The residue was partitioned between ethyl acetate and water, and was filtered through Celite. The aqueous layer was acidified by 4N hydrochloric acid to be pH 2 and extracted with ethyl acetate. The organic layer was washed with brine, dried over magnesium sulfate, and concentrated in vacuo. The residue was purified by Reverse Phase HPLC (eluent: 30%-80% acetonitrile in water containing 0.1% trifluoroacetic acid, linear gradient method) to give [(2S)-2-[5-[4-(2-oxazolyl)-phenyl]thiophen-2-yl]-1,1-dioxo-3,4,5,6-tetrahydro-2H-thiopyran-2-yl]acetic acid (190 mg) as a solid.

NMR (DMSO-d₆, δ): 1.68-2.10 (4H, m), 2.38-2.49 (1H, m), 2.84 (1H, d, J=11 Hz), 3.09-3.27 (2H, d, J=15 Hz), 3.47-3.63 (2H, m), 7.24 (1H, d, J=4 Hz), 7.41 (1H, s), 7.61 (1H, d, J=4 Hz), 7.83 (2H, d, J=8 Hz), 8.02 (2H, d, J=8 Hz), 8.25 (1H, s)

MS (ESI+): 416 (M+H)

The following compounds were obtained in a similar manner to that of Example 11.

### EXAMPLE 12

[(2S)-2-[5-(5-Benzofuranyl)thiophen-2-yl]-1,1-dioxo-3,4,5,6-tetrahydro-2H-thiopyran-2-yl]acetic acid (37.5 mg)

NMR (DMSO-d₆, δ): 1.69-2.20 (4H, m), 2.35-2.60 (1H, m), 2.77-3.00 (1H, m), 3.13-3.73 (4H, m), 7.00 (1H, br), 7.20 (1H, br), 7.46 (1H, br), 7.64 (2H, m), 7.94 (1H, br), 8.04 (1H, br), 12.56 (1H, br)

MS (m/z): 389 (M−H)

### EXAMPLE 13

[(2S)-2-[5-[4-(3,4-Methylenedioxyphenyl)thiophen-2-yl]-1,1-dioxo-3,4,5,6-tetrahydro-2H-thiopyran-2-yl]acetic acid (245 mg) from 3,4-methylenedioxybenzeneboronic acid

NMR (DMSO-d₆, 5): 1.70-2.07 (4H, m), 2.35-2.48 (1H, m), 2.72-2.86 (1H, m), 3.16 (2H, d, J=15 Hz), 3.44-3.60 (2H, m), 6.06 (2H, s), 6.95 (1H, d, J=8 Hz), 7.07-7.65 (2H, m), 7.26 (1H, s), 7.36 (1H, d, J=4 Hz)

MS (ESI−): 393 (M−H)

### EXAMPLE 14

[(2S)-2-[5-(Benzothiophen-2-yl)thiophen-2-yl]-1,1-dioxo-3, 4,5,6-tetrahydro-2H-thiopyran-2-yl] acetic acid (120 mg) from benzothiophene-2-boronic acid

NMR (DMSO-d₆, δ): 1.72-2.11 (4H, m), 2.35-2.49 (1H, m), 2.72-2.89 (1H, m), 3.12-3.37 (2H, m), 3.45-3.65 (2H, m), 7.21 (1H, d, J=4 Hz), 7.32-7.45 (3H, m), 7.69 (1H, s), 7.83 (2H, d, J=8 Hz), 7.96 (2H, d, J=8 Hz)

MS (ESI−): 405 (M−H)

### EXAMPLE 15

[(2S)-2-[5-(4-n-Pentylphenyl)thiophen-2-yl1-1₁₁-dioxo-3,4,5,6-tetrahydro-2H-thiopyran-2-yl]acetic acid (125 mg) from 4-n-pentylbenzeneboronic acid

NMR (DMSO-d₆, δ): 0.86 (3H, t, J=8 Hz), 1.21-1.37 (4H, m), 1.51-1.64 (2H, m), 1.70-2.08 (4H, m), 2.36-2.48 (1H, m), 2.58 (2H, t, J=8 Hz), 2.75-2.87 (1H, m), 3.11-3.23 (2H, m), 3.44-3.62 (2H, m), 7.16 (1H, d, J=4 Hz), 7.24 (2H, d, J=8 Hz), 7.41 (1H, d, J=4 Hz), 7.55 (2H, d, J=8 Hz)

MS (ESI−): 419 (M−H)

### EXAMPLE 16

[(2S)-2-[5-(2,3-Dihydrobenzofuran-5-yl)thiophen-2-yl]-1,1-dioxo-3,4,5,6-tetrahydro-2H-thiopyran-2-yl]acetic acid (166 mg)

NMR (DMSO-d₆, δ): 1.68-2.08 (4H, m), 2.34-2.49 (1H, m), 2.74-2.86 (1H, m), 3.07-3.28 (4H, m), 3.46-3.62 (2H, m), 4.56 (2H, t, J=8 Hz), 6.80 (1H, d, J=8 Hz), 7.11 (1H, d, J=4 Hz), 7.29 (1H, d, J=4 Hz), 7.31-7.42 (1H, m), 7.53 (1H, s)

MS (ESI−): 419 (M−H)

### EXAMPLE 17

[(2S)-2-[5-(4-Benzyloxyphenyl)thiophen-2-yl]-1,1-dioxo-3,4,5,6-tetrahydro-2H-thiopyran-2-yl]acetic acid (150 mg) from 4-benzyloxybenzeneboronic acid

NMR (DMSO-d₆, δ): 1.72-2.15 (4H, m), 2.40-2.60 (1H, m), 2.78-2.92 (1H, m), 3.12-3.26 (2H, m), 3.46-3.66 (2H, m), 5.14 (2H, s), 7.07 (2H, d, J=8 Hz), 7.14 (1H, d, J=3 Hz), 7.34 (1H, d, J=3 Hz), 7.28-7.48 (5H, m), 7.58 (2H, d, J=8 Hz)

The following compounds were obtained in a similar manner to that of Example 1.

### EXAMPLE 18

[(2S)-2-[5-(3-Quinolyl)thiophen-2-yl]-1,1-dioxo-3,4,5,6-tetrahydro-2H-thiopyran-2-yl]acetic acid (137 mg)

NMR (DMSO-d₆, δ): 1.74-2.14 (4H, m), 2.40-2.60 (1H, m), 2.80-2.95 (1H, m), 3.14-3.32 (2H, m), 3.52-3.68 (2H, m), 7.31 (1H, d, J=3 Hz), 7.68 (1H, t, J=7 Hz), 7.82 (2H, m), 8.03-8.14 (2H, m), 8.72 (1H, d, J=2 Hz), 9.33 (1H, d, J=2 Hz)

MS (m/z): 402 (M+H)

### EXAMPLE 19

[(2S)-2-[5-(6-Quinolyl)thiophen-2-yl]-1,1-dioxo-3,4,5,6-tetrahydro-2H-thiopyran-2-yl]acetic acid (172 mg)

NMR (DMSO-d₆, δ): 1.73-2.17 (4H, m), 2.45-2.55 (1H, m), 2.80-2.95 (1H, m), 3.15-3.30 (2H, m), 3.48-3.68 (2H, m), 7.27 (1H, d, J=3 Hz), 7.65-7.75 (2H, m), 8.09 (1H, d, J=8 Hz), 8.18 (1H, d, J=8 Hz), 8.35 (1H, s), 8.58 (1H, d, J=8 Hz), 8.97 (1H, m)

### EXAMPLE 20

To the crude mixture of 4′-cyano-4-biphenylylboronic acid pinacol cyclic ester obtained in Preparation 2-2) was added a solution of 2-(trimethylsilyl)ethyl [(2S)-2-(5-bromothiophen-2-yl)-1,1-dioxo-3,4,5,6-tetrahydro-2H-thiopyran-2-yl]acetate (100 mg) in dioxane (2 ml), dichlorobis(triphenylphosphine)palladium(II) (5 mg) and 2M sodium carbonate (2 ml) at ambient temperature under nitrogen gas atmosphere. The mixture was heated at 80° C. for 3 hours. The cooled reaction mixture was partitioned between ethyl acetate and water, and was filtered through Celite. The organic layer was separated, washed with brine, dried over magnesium sulfate, and evaporated in vacuo. The residue was purified by flash silica gel chromatography (silica gel, 40 ml) eluting with hexane-ethyl acetate=5-1, 3-1, and 2-1 and followed by trituration with isopropyl ether to give 2-(trimethylsilyl)ethyl [(2S)-2-[5-(4′-cyano-4-biphenylyl)thiophen-2-yl]-1,1-dioxo-3,4,5,6-tetrahydro-2H-thiopyran-2-yl]acetate as a colorless solid (121 mg).

NMR (CDCl₃, δ): -0.04 (9H, s), 0.77-0.86 (2H, m), 1.76-2.24 (4H, m), 2.72-2.91 (2H, m), 3.03-3.21 (3H, m), 3.44 (1H, d, J=15 Hz), 3.97-4.09 (2H, m), 7.26 (1H, d, J=4 Hz), 7.31 (1H, d, J=4 Hz), 7.60 (2H, d, J=BHz), 7.65-7.76 (6H, m)

MS (ESI+): 552 (M+1)

The following compounds were obtained in a similar manner to that of Example 20.

### EXAMPLE 21

2-(Trimethylsilyl)ethyl [(2S)-2-[5-(4′ethoxy-4-biphenylyl)thiophen-2-yl]-1,1-dioxo-3,4,5,6-tetrahydro-2H-thiopyran-2-yl]acetate (88 mg)

NMR (CDCl₃, δ): -0.05 (9H, s), 0.75-0.86 (2H, m), 1.44 (3H, t, J=8 Hz), 1.75-2.25 (4H, m), 2.68-2.93 (2H, m), 2.96-3.21 (3H, m), 3.42 (1H, d, J=15 Hz), 3.93-4.14 (4H, m), 6.96 (2H, d, J=8 Hz), 7.20-7.31 (2H, m, overlapped with CDCl₃), 7.49-7.67 (6H, m)

### EXAMPLE 22

2-(Trimethylsilyl)ethyl [(2S)-2-[5-[4-(5-oxazolyl)phenyl]thiophen-2-yl]-1,1-dioxo-3,4,5,6-tetrahydro-2H-thiopyran-2-yl]acetate (93 mg)

NMR (CDCl₃, δ): -0.05 (9H, s), 0.75-0.86 (2H, m), 1.75-2.25 (4H, m), 2.70-2.91 (2H, m), 3.03-3.21 (3H, m), 3.43 (1H1, d, J=15 Hz), 3.94-4.14 (2H, m), 7.21-7.32 (2H, m), 7.37 (1H, s), 7.61-7.68 (4H, m), 7.92 (1H, s)

MS (ESI+): 518 (M+1)

### EXAMPLE 23

2-(Trimethylsilyl)ethyl [(2S)-2-[5-(4-n-butylphenyl)thiophen-2-yl]-1,1-dioxo-3,4,5,6-tetrahydro-2H-thiopyran-2-yl]acetate (30 mg) from 4-n-butylbenzeneboronic acid

NMR (CDCl₃, δ): -0.05 (9H, s), 0.74-0.85 (2H, m), 0.93 (3H, t, J=8 Hz), 1.20-1.44 (2H, m), 1.49-1.66 (2H, m), 1.75-2.25 (4H, m), 2.60 (2H, t, J=8 Hz), 2.69-2.93 (2H, m), 2.99-3.20 (3H, m), 3.41 (1H, d, J=15 Hz), 3.95-4.07 (2H, m), 7.14-7.31 (4H, m), 7.49 (2H, d, J=8 Hz)

MS (ESI+): 507 (M+1)

### EXAMPLE 24

2-(Trimethylsilyl)ethyl [(2S)-2-[5-(4-cyclohexylphenyl)thiophen-2-yl]-1,1-dioxo-3,4,5,6-tetrahydro-2H-thiopyran-2-yl]acetate (56.1 mg) from 4-cyclohexylbenzeneboronic acid

NMR (CDCl₃, δ): -0.05 (9H, s), 0.79 (2H, m), 1.25-1.49 (4H, m), 1.74-2.18 (10H, m), 2.50 (1H, m), 2.70-2.91 (2H, m), 3.01-3.19 (3H, m), 3.40 (1H, d, J=15 Hz), 4.00 (2H, m), 7.18-7.24 (4H, m), 7.50 (2H, d, J=8 Hz)

MS (ESI+): 533.23 (MH)

### EXAMPLE 25

2-(Trimethylsilyl)ethyl [(2S)-2-[5-(2-benzofuranyl)thiophen-2-yl]-1,1-dioxo-3,4,5,6-tetrahydro-2H-thiopyran-2-yl]acetate (53.7 mg) from benzofuran-2-boronic acid

NMR (CDCl₃, δ): -0.04 (9H, s), 0.81 (2H, m), 1.81-2.20 (3H, m), 2.72-2.92 (2H, m), 3.04-3.20 (3H, m), 3.31 (1H, d, J=15 Hz), 4.00-4.10 (1H, m), 7.19-7.31 (3H, m), 7.41 (1H, d, J=4 Hz), 7.47 (1H, d, J=8 Hz), 7.55 (1H, dd, J=2 and 7 Hz)

### EXAMPLE 26

2-(Trimethylsilyl)ethyl [(2S)-2-[5-(4-hydroxyphenyl)thiophen-2-yl]-1,1-dioxo-3,4,5,6-tetrahydro-2H-thiopyran-2-yl]acetate (132 mg) from 4-hydroxybenzeneboronic acid

NMR (CDCl₃, δ): -0.04 (9H, s), 0.81 (2H, m), 1.81-2.19 (4H, m), 2.71-2.87 (3H, m), 3.03-3.21 (3H, m), 3.42 (1H, d, J=15 Hz), 4.03 (1H, s), 5.15 (1H, s), 6.79 (1H, d, J=9 Hz), 7.09 (1H, d, J=4 Hz), 7.20 (1H, d, J=4 Hz), 7.42 (1H, d, J=9 Hz)

### EXAMPLE 27

2-(Trimethylsilyl)ethyl [(2S)-2-[5-(4-biphenylyl)thiophen-2-yl]-1,1-dioxo-3,4,5,6-tetrahydro-2H-thiopyran-2-yl]acetate (30.6 mg) from 4-biphenylboronic acid

NMR (CDCl₃, δ): -0.04 (9H, s), 0.82 (2H, m), 1.83-2.18 (4H, m), 2.73-2.92 (2H, m), 3.03-3.20 (3H, m), 3.43 (1H, d, J=15 Hz), 4.03 (2H, m), 7.29 (2H, m), 7.36 (1H, t, J=7 Hz), 7.45 (1H, t, J=8 Hz), 7.59-7.68 (6H, m)

### EXAMPLE 28

2-(Trimethylsilyl)ethyl 1(2S)-2-[5-(2-naphthyl)thiophen-2-yl]-1,1-dioxo-3,4,5,6-tetrahydro-2H-thiopyran-2-yl]acetate (64.8 mg)

NMR (CDCl₃, δ): -0.05 (9H, s), 0.82 (3H, m), 1.85-2.21 (4H, m), 2.74-2.95 (2H, m), 3.04-3.22 (3H, m), 3.45 (1H, d, J=15 Hz), 4.04 (2H, m), 7.29 (1H, d, J=4 Hz), 7.38 (1H, d, J=4 Hz), 7.44-7.52 (2H, m), 7.73-7.85 (3H, m), 7.80-7.85 (3H, m), 8.05 (1H, s)

### EXAMPLE 29

2-(Trimethylsilyl)ethyl [(2S)-2-[5-(3-aminophenyl)thiophen-2-yl]-1,1-dioxo-3,4,5,6-tetrahydro-2H-thiopyran-2-yl]acetate (174 mg) from 3-aminobenzeneboronic acid hydrochloride

NMR (CDCl₃, δ): -0.04 (9H, s), 0.88 (2H, m), 1.76-2.18 (4H, m), 2.71-2.90 (2H, m), 3.01-3.19 (3H, m), 3.40 (1H, d, J=15 Hz), 3.69 (2H, br s), 4.01 (2H, m), 6.62 (1H, d, J=8 Hz), 6.91 (1H, d, J=2 Hz), 6.99 (1H, d, J=8 Hz), 7.12-7.19 (3H, m)

MS (ESI+): 466.31 (MH)

### EXAMPLE 30

2-(Trimethylsilyl)ethyl [(2S)-2-[5-(3-quinolyl)thiophen-2-yl]-1,1-dioxo-3,4,5,6-tetrahydro-2H-thiopyran-2-yl]acetate (350 mg)

NMR (CDCl₃, δ): -0.04 (9H, s), 0.79-0.96 (2H, m), 1.81-2.31 (4H, m), 2.76-3.00 (2H, m), 3.06-3.26 (2H, m), 3.17 (1H, d, J=15 Hz), 3.46 (1H, d, J=15 Hz), 4.00-4.14 (2H, m), 7.30 (1H, d, J=3 Hz), 7.43 (1H, d, J=3 Hz), 7.55 (1H, t, J=8 Hz), 7.69 (1H, t, J=8 Hz), 7.81 (1H, d, J=8 Hz), 8.09 (1H, d, J=8 Hz), 8.28 (1H, s), 9.18 (1H, s)

MS (m/z): 502 (M+H)

### EXAMPLE 31

2-(Trimethylsilyl)ethyl [(2S)-2-[5-(6-quinolyl)thiophen-2-yl]-1,1-dioxo-3,4,5,6-tetrahydro-2H-thiopyran-2-yl]acetate (372 mg).

NMR (CDCl₃, δ): -0.04 (9H, s), 0.77-0.91 (2H, m), 1.81-2.11 (2H, m), 2.12-2.31 (2H, m), 2.78-2.97 (2H, m), 3.06-3.26 (2H, m), 3.14 (1H, d, J=15 Hz), 3.46 (1H, d, J=15 Hz), 4.00-4.10 (2H, m), 7.31 (1H, d, J=3 Hz), 7.39-7.58 (3H, m), 7.62-7.71 (1H, m), 7.93-8.01 (1H, m), 8.08-8.17 (1H, m), 8.87 (1H, m)

MS (m/z): 502 (M+H)

### EXAMPLE 32

A solution of 2-(trimethylsilyl)ethyl [(2S)-2-[5-(3-aminophenyl)thiophen-2-yl]-1,1-dioxo-3,4,5,6-tetrahydro-2H-thiopyran-2-yl]acetate (64.5 mg) and n-propylisocyanate (13.0 mg) in chloroform (1 ml) was stirred for 1 hour at room temperature. The resulting mixture was stirred for further 25 hours at room temperature after adding n-propylisocyanate (10.0 mg). Ethyl acetate (10 ml) was added to the mixture, and the solution was washed with 3% citric acid, brine, dried, and evaporated to give an yellowish gum. Silica gel column chromatography (eluent: ethyl acetate/hexane=1/3 to 1/0) afforded 2-(trimethylsilyl)ethyl [(2S)-2-(5-[3-(3-n-ropylureido)phenyl]thiophen-2-yl]-1,1-dioxo-3,4,5,6-tetrahydro-2H-thiopyran-2-yl]acetate as a white solid (57.7 g).

NMR (CDCl₃, δ): -0.04 (9H, s), 0.82 (2H, m), 0.93 (3H, t, J=7 Hz), 1.77-2.19 (4H, m), 2.71-2.90 (2H, m), 2.79 (2H, m), 3.09-3.26 (5H, m), 3.41 (1H, d, J=15 Hz), 4.02 (2H, m), 4.84 (1H, m), 6.47 (1H, s), 7.21 (4H, m), 7.49 (1H, s)

### EXAMPLE 33

To a solution of 2-(trimethylsilyl)ethyl [(2S)-2-[5-(4-hydroxyphenyl)thiophen-2-yl]-1,1-dioxo-3,4,5,6-tetrahydro-2H-thiopyran-2-yl]acetate (20.2 mg) and n-propyliodide (8.83 mg) in dimethylformamide (0.5 ml) was added pottasium carbonate (12.0 mg). The resulting mixture was stirred for 5 hours at room temperature. The mixture was stirred for further 2 hours after adding n-propyliodide (6.7 mg). Ethyl acetate (10 ml) was added to the mixture, and the solution was washed with 1N hydrochloric acid (5 ml), water, brine, dried, and evaporated. Silica gel column chromatography (eluent: ethyl acetate/hexane=1/4 to 1/2) afforded 2-(trimethylsilyl)ethyl [(2S)-2-[5-(4-n-propyloxyphenyl)thiophen-2-yl]-1,1-dioxo-3,4,5,6-tetrahydro-2H-thiopyran-2-yl]acetate as an yellowish gum (13.8 g).

NMR (CDCl₃, δ): -0.07 (9H, s), 0.78 (2H, m), 1.02 (3H, t, J=8 Hz), 1.74-2.16 (6H, m), 2.67-2.93 (2H, m), 2.99-3.16 (3H, m), 3.38 (1H, d, J=15 Hz), 3.91 (2H, t, J=7 Hz), 4.00 (2H, m), 6.86 (2H, d, J=9 Hz), 7.09 (1H, d, J=4 Hz), 7.18 (1H, d, J=4 Hz), 7.47 (2H, d, J=9 Hz)

The following compounds were obtained in a similar manner to that of Example 94.

### EXAMPLE 34

((2S)-1,1-Dioxo-2-{5-[4-(pyridin-4-yl)phenyl]-thiophen-2-yl}-3,4,5,6-tetrahydro-2H-thiopyran-2-yl)acetic acid

NMR (DMSO-d₆, δ): 1.73-2.11 (4H, m), 2.40-2.50 (1H, m), 2.78-2.90 (1H, m), 3.15-3.26 (2H, m), 3.50-3.65 (2H, m), 7.26 (1H, d, J=4 Hz), 7.68 (1H, d, J=4 Hz), 7.88 (2H, d, J=8 Hz), 8.05 (2H, d, J=8 Hz), 8.18-8.29 (2H, m), 8.87 (2H, br)

MS (ESI+): 428 (M+H)

### EXAMPLE 35

((2S)-1,1-Dioxo-2-{5-[4-(thiophen-3-yl)phenyl]-thiophen-2-yl}-3,4,5,6-tetrahydro-2H-thiopyran-2-yl)acetic acid

NMR (DMSO-d₃, δ): 1.71-2.06 (4H, m), 2.40-2.50 (1H, m), 2.78-2.88 (1H, m), 3.12-3.26 (2H, m), 3.46-3.62 (2H, m), 7.16-7.24 (1H, m), 7.48-7.55 (1H, m), 7.54-7.83 (7H, m)

### EXAMPLE 36

((2S)-1,1-Dioxo-2-[5-[4-(thiazol-2-yl)phenyl]-thiophen-2-yl}-3,4,5,6-tetrahydro-2H-thiopyran-2-yl)acetic acid

NMR (DMSO-d₆, δ): 1.70-2.10 (4H, m), 2.40-2.53 (1H, m), 2.78-2.89 (1H, m), 3.12-3.28 (2H, m), 3.46-3.64 (2H, m), 7.23 (1H, d, J=4 Hz), 7.59 (1H, d, J=4 Hz), 7.75-7.86 (3H, m), 7.92-8.04 (3H, m)

MS (ESI−): 432 (M−H)

### EXAMPLE 37

((2S)-1,1-Dioxo-2-[(5-[4-(pyridin-3-yl)phenyl]-thiophen-2-yl)-3,4,5,6-tetrahydro-2H-thiopyran-2-yl)acetic acid

NMR (DMSO-d₆, δ): 1.70-2.11 (4H, m), 2.36-2.50 (1H, m), 2.76-2.70 (1H, m), 3.12-3.28 (2H, m), 3.47-3.65 (2H, m), 7.24 (1H, d, J=4 Hz), 7.62 (1H, d, J=4 Hz), 7.75-7.95 (5H, m), 8.57 (1H, d, J=8 Hz), 8.76 (1H, br), 9.16 (1H, br)

MS (ESI+): 428 (M+H)

### EXAMPLE 38

((2S)-1,1-Dioxo-2-(5-[4-(pyridin-2-yl)phenyl]-thiophen-2-yl}-3,4,5,6-tetrahydro-2H-thiopyran-2-yl)acetic acid

NMR (DMSO-d₆, δ): 1.70-2.10 (4H, m), 2.34-2.48 (1H, m), 2.76-2.90 (1H, m), 3.11-3.26 (2H, m), 3.51-3.65 (2H, m), 7.23 (1H, d, J=4 Hz), 7.33-7.42 (1H, m), 7.58 (1H, d, J=4 Hz), 7.79 (2H, d, J=8 Hz), 7.86-7.95 (1H, m), 8.00 (1H, d, J=8 Hz), 8.16 (2H, d, J=8 Hz), 8.68 (1H, d, J=4 Hz)

MS (ESI+): 428 (M+H)

### EXAMPLE 39

{(2S)-2-[5-(Benzothiophen-5-yl)thiophen-2-yl]-1,1-dioxo-3,4,5,6-tetrahydro-2H-thiopyran-2-yl} acetic acid

NMR (DMSO-d₆, δ): 1.74-2.10 (4H, m), 2.82-2.89 (2H, m), 3.19 (2H, d, J=15 Hz), 3.58 (2H, d, J=15 Hz), 7.20 (1H, d, J=4 Hz), 7.52 (1H, dd, J=5, 4 Hz), 7.68 (1H, dd. J=8, 1 Hz), 7.82 (2H, d, J=7 Hz), 8.06 (2H, d, J=8 Hz), 8.19 (1H, d, J=1 Hz), 8.28 (1H, s), 8.55 (1H, s)

MS (m/z): 405 (M⁺−H, bp)

### EXAMPLE 40

{(2S)-1,1-Dioxo-2-[5-(6-methoxycarbonylnaphthalen-2-yl)thiophen-2-yl]-3,4,5,6-tetrahydro-2H-thiopyran-2-yl} acetic acid

NMR (DMSO-d₆, δ): 1.75-2.10 (4H, m), 2.81-2.90 (2H, m), 3.22 (2H, d, J=15 Hz), 3.61 (2H, d, J=15 Hz), 3.92 (3H, s), 7.26 (1H, d, J=6 Hz), 7.72 (1H, d, J=56 Hz), 7.94-8.00 (2H, m), 8.10 (1H, d, J=8 Hz), 8.19 (1H, d, J=8 Hz), 8.29 (1H, s), 8.63 (1H, s), 8.55 (1H, s)

MS (m/z): 458 (M⁺−H), 175 (bp)

### EXAMPLE 41

((2S)-1,1-Dioxo-2-[5-(6-methoxynaphthalen-2-yl)thiophen-2-yl]-3,4,5,6-tetrahydro-2H-thiopyran-2-yl)acetic acid

NMR (DMSO-d₆, δ): 1.75-2.09 (4H, m), 2.80-2.89 (2H, m), 3.20 (2H, d, J=15 Hz), 3.57 (2H, d, J=15 Hz), 3.89 (3H, s), 7.16-7.20 (2H, m), 7.34 (1H, d, J=2 Hz), 7.54(1H, d, J=4 Hz), 7.78(1H, dd, J=8, 2 Hz), 7.86(1H, d, J=8 Hz), 7.90(1H, s), 8.10(1H, d, J=2 Hz)

MS (m/z): 430 (M⁺−H), 137 (bp)

### EXAMPLE 42

{(2S)-1,1-Dioxo-2-[5-(6-formylnaphthalen-2-yl)thiophen-2-yl]-3,4,5,6-tetrahydro-2H-thiopyran-2-yl}acetic acid

NMR (CDCl₃, δ): 1.80-2.05 (2H, m), 2.12-2.24 (2H, m), 2.75-2.89 (2H, m), 3.05-3.19(3H, m), 3.24(1H, d, J=15 Hz), 3.45 (1H, d, J=15 Hz), 7.33 (1H, d, J=2 Hz), 7.45 (1H, d, J=2 Hz), 7.80-8.00(4H, m), 8.09 (1H, s), 8.30 (1H, s), 10.15 (1H, s)

MS (m/z): 427 (M⁺−H, bp)

### EXAMPLE 43

((2S)-1,1-Dioxo-2-{5-[6-(oxazol-5-yl)naphthalen-2-yl]thiophen-2-yl}-3,4,5,6-tetrahydro-2H-thiopyran-2-yl)acetic acid

mp: 260-265° C.

NMR (DMSO-d₆, δ): 1.73-2.10 (4H, m), 2.80-2.90 (2H, m), 3.20 (2H, d, J=15 Hz), 3.60 (2H, d, J=15 Hz), 7.24 (1H, d, J=2 Hz), 7.65 (1H, d, J=2 Hz), 7.85 (1H, s), 7.90 (2H, d, J=8 Hz), 8.09 (2H, t, J=8 Hz), 8.23 (1H, s), 8.28 (1H, s), 8.55 (1H, s)

MS (m/z): 466 (M⁺−H), 82 (bp)

### EXAMPLE 44

{(2S)-1,1-Dioxo-2-(5-(6-hydroxynaphthalen-2-yl)thiophen-2-yl]-3,4,5,6-tetrahydro-2H-thiopyran-2-yl} acetic acid

NMR (DMSO-d₆, δ): 1.73-2.10 (4H, m), 2.70-2.90 (2H, m), 3.18 (2H, d, J=15 Hz), 3.57 (2H, d, J=15 Hz), 7.08-7.12 (2H, s), 7.19 (1H, d, J=2 Hz), 7.50 (1H, d, J=2 Hz), 7.71 (2H, s), 7.83 (1H, d, J=7 Hz), 8.04 (1H, s), 9.85 (1H, broad s)

MS (m/z): 415 (M⁺−H, bp)

### EXAMPLE 45

{(2S)-2-[5-(Benzoxazol-2-yl)thiophen-2-yl]-1,1-dioxo-3,4,5,6-tetrahydro-2H-thiopyran-2-yl} acetic acid

NMR (CDCl₃, δ): 1.83-2.09 (2H, m), 2.14-2.24 (2H, m), 2.84-2.88 (2H, m), 3.10-3.17 (2H, m), 3.23-3.31 (1H, m), 3.51-3.60 (1H, m), 7.34 (2H, dd, J=7, 2 Hz), 7.43 (1H, d, J=2 Hz), 7.50-7.54 (1H, m), 7.72-7.76 (1H, m), 7.84 (1H, d, J=2 Hz)

MS (m/z): 390 (M⁺−H, bp)

### EXAMPLE 46

{(2S)-2-[5-(Benzothiophen-3-yl)thiophen-2-yl]-1,1-dioxo-3,4,5,6-tetrahydro-2H-thiopyran-2-yl} acetic acid

NMR (DMSO-d₆, δ): 1.75-2.10 (4H, m), 2.72-2.87 (2H, m), 3.20 (2H, d, J=15 Hz), 3.54 (2H, d, J=15 Hz), 7.28-7.34 (2H, m), 7.39-7.44 (2H, m), 7.53 (1H, s), 7.90 (1H, dd, J=7.5, 1 Hz), 8.13 (1H, dd, J=7.5, 1 Hz)

MS (m/z): 405(M⁺−H, bp)

### EXAMPLE 47

{(2S)-2-[5-(6-Cyanonaphthalen-2-yl)thiophen-2-yl]-1,1-dioxo-3,4,5,6-tetrahydro-2H-thiopyran-2-yl}acetate

mp: 235-238° C.

NMR (DMSO-d₆, δ): 1.74-2.10 (4H, m), 2.80-2.90 (2H, m), 3.20 (2H, d, J=15 Hz), 3.60 (2H, d, J=lSHz), 7.27 (1H, d, J=4 Hz), 7.73 (1H, d, J=4 Hz), 7.8 (1H, d, J=8 Hz), 8.00-8.19 (3H, m), 8.34 (1H, s), 8.57 (1H, s)

MS (m/z): 424(M⁺−H, bp)

### EXAMPLE 48

{(2S)-1,1-Dioxo-2-[5-(6-ethoxynaphthalen-2-yl)thiophen-2-yl]-3,4,5,6-tetrahydro-2H-thiopyran-2-yl} acetic acid

mp: 219-222° C.

NMR (DMSO-d₆, δ): 1.39 (3H, t, J=7 Hz), 1.73-2.10 (4H, m), 2.41 (3H, s), 2.72-2.89 (2H, m), 3.17 (2H, d, J=15 Hz), 3.57 (2H, d, J=15 Hz), 4.16 (2H, q, J=7 Hz), 7.15-7.20 (2H, m), 7.33 (1H, d, J=2 Hz), 7.54(1H, d, J=2 Hz), 7.75-7.90 (3H, m), 8.10 (1H, s)

MS (m/z): 443(M⁺−H), 118 (bp)

### EXAMPLE 49

{(2S)-1,1-Dioxo-2-[5-(1-methyl-2,3-dihydro-2-oxo-indol-5-yl)thiophen-2-yl]-3,4,5,6-tetrahydro-2H-thiopyran-2-yl} acetic acid

NMR (CDCl₃, δ): 1.75-2.03 (2H, m), 2.11-2.22 (2H, m), 2.77-2.85 (2H, m), 3.07-3.20 (3H, m), 3.21 (3H, s), 3.46 (2H, s), 3.50 (1H, d, J=15 Hz), 6.79 (1H, d, J=7.5 Hz), 7.15 (1H, d, J=2 Hz), 7.23 (1H, d, J=2 Hz), 7.44 (1H, s), 7.50 (1H, d, J=7.5 Hz)

MS (m/z): 419 (M⁺−H), 127(bp)

### EXAMPLE 50

{(2S)-2-[5-(5-Methylbenzothiophen-2-yl)thiophen-2-yl]-1,1-dioxo-3,4,5,6-tetrahydro-2H-thiopyran-2-yl} acetic acid

mp: 209-213° C.

NMR (DMSO-d₆, δ): 1.73-2.10 (4H, m), 2.41 (3H, s), 2.72-2.88 (2H, m), 3.18 (2H, d, J=15 Hz), 3.59 (2H, d, J=15 Hz), 7.14 (1H, d, J=8 Hz), 7.15 (1H, d, J=2 Hz), 7.39 (1H, d, J=8 Hz), 7.60 (2H, d, J=8 Hz), 7.83 (1H, d, J=8 Hz)

MS (m/z): 419(M⁺−H, bp)

### EXAMPLE 51

{(2S)-2-[5-(5-Methoxybenzothiophen-2-yl)thiophen-2-yl]-1,1-dioxo-3,4,5,6-tetrahydro-2H-thiopyran-2-yl} acetic acid

mp: 215-218° C.

NMR (DMSO-d₆, δ): 1.72-2.09 (4H, m), 2.70-2.88 (2H, m), 3.18 (2H, d, J=15 Hz), 3.58 (2H, d, J=15 Hz), 3.82 (3H, s), 6.99 (1H, dd, J=8, 2 Hz), 7.19 (1H, d, J=8 Hz), 7.33-7.40 (2H, m), 7.60 (1H, s), 7.83 (1H, d, J=8 Hz)

MS (m/z): 435(M⁺−H), 391(bp)

### EXAMPLE 52

{(2S)-2-[5-(6-Methoxybenzothiophen-2-yl)thiophen-2-yl]-1,1-dioxo-3,4,5,6-tetrahydro-2H-thiopyran-2-yl} acetic acid

mp: 232-235° C.

NMR (DMSO-d₆, δ): 1.72-2.10 (4H, m), 2.71-2.86 (2H, m), 3.13-3.24 (2H, m), 3.57 (2H, d, J=15 Hz), 3.83 (3H, s), 6.93 (1H, d, J=8 Hz), 7.00 (1H, dd, J=B, 2 Hz), 7.17 (1H, t, J=2 Hz), 7.33 (1H, d, J=2 Hz), 7.55 (1H, d, J=2 Hz), 7.57 (1H, s), 7.71 (1H, d, J=8 Hz)

MS (m/z): 435(M⁺−H, bp)

### EXAMPLE 53

{(2S)-2-[5-(5-Fluorobenzothiophen-2-yl)thiophen-2-yl]-1,1-dioxo-3,4,5,6-tetrahydro-2H-thiopyran-2-yl} acetic acid

mp: 219-221° C.

NMR (DMSO-d₆, δ): 1.75-2.05 (4H, m), 2.71-2.87 (2H, m), 3.14-3.24 (2H, m), 3.59 (2H, d, J=15 Hz), 7.21 (1H, d, J=4 Hz), 7.24-7.28 (1H, m), 7.44 (1H, d, J=4 Hz), 7.65 (1H, dd, J=8, 4 Hz), 7.67 (1H, s), 7.99-8.04 (1H, m)

MS (m/z): 423(M⁺−H, bp)

### EXAMPLE 54

[(2S)-1,1-Dioxo-2-(5-{6-[methylcarbamoyl)naphthalen-2-yl}thiophen-2-yl)-3,4,5,6-tetrahydro-2H-thiopyran-2-yl]acetic acid

mp: 230-232° C.

NMR (DMSO-d₆, δ): 1.75-2.10 (4H, m), 2.70-2.91 (2H, m), 2.84 (3H, d, J=5 Hz), 3.20 (2H, d, J=15 Hz), 3.59 (2H, d, J=15 Hz), 7.25 (1H, d, J=2 Hz), 7.68 (1H, d, J=2 Hz), 7.89-7.95 (2H, m), 8.02-8.07 (2H, m), 8.24 (1H, s), 8.41 (1H, s), 8.60 (1H, d, J=5 Hz)

MS (m/z): 456 (M⁺−H, bp)

### EXAMPLE 55

((2S)-1,1-Dioxo-2-(5-(4-(pyrazin-2-yl)phenyl)thiophen-2-yl)-3,4,5,6-tetrahydro-2H-thiopyran-2-yl)acetic acid

NMR (DMSO-d₆, δ): 1.72-2.15 (4H, m), 2.40-2.50 (1H, m), 2.78-2.95 (1H, m), 3.16-3.25 (2H, m), 3.48-3.70 (2H, m), 7.24 (1H, d, J=3 Hz), 7.62 (1H, d, J=3 Hz), 7.83 (2H, d, J=8 Hz), 8.22 (2H, d, J=8 Hz), 8.62 (1H, d, J=2 Hz), 8.73 (1H, d, J=2 Hz), 9.31 (1H, s)

MS (m/z): 427 (M−H)

### EXAMPLE 56

((2S)-1,1-Dioxo-2-(5-(4-(pyrimidin-5-yl)phenyl)-thiophen-2-yl)-3,4,5,6-tetrahydro-2H-thiopyran-2-yl)acetic acid

NMR (DMSO-d₆, δ): 1.73-2.15 (4H, m), 2.42-2.55 (1H, m), 2.80-2.93 (1H, m), 3.15-3.28 (2H, m), 3.47-3.55 (2H, m), 7.23 (1H, d, J=3 Hz), 7.60 (1H, d, J=3 Hz), 7.82 (2H, d, J=8 Hz), 7.90 (2H, d, J=8 Hz), 9.20 (3H, m)

### EXAMPLE 57

((2S)-2-(5-(4-(2-Methylthiazol-4-yl)phenyl)thiophen-2-yl)-1,1-dioxo-3,4,5,6-tetrahydro-2H-thiopyran-2-yl)acetic acid

NMR (DMSO-d₆, δ): 1.72-2.13 (4H, m), 2.40-2.55 (1H, m), 2.73 (3H, s), 2.79-2.93 (1H, m), 3.14-3.26 (2H, m), 3.47-3.62 (2H, m), 7.20 (1H, d, J=3 Hz), 7.53 (1H, d, J=3 Hz), 7.71 (2H, d, J=8 Hz), 7.98 (2H, d, J=8 Hz), 8.00 (1H, s)

MS (m/z): 448 (M+H)

### EXAMPLE 58

((2S)-2-(5-(4-(Isoxazol-5-yl)phenyl)thiophen-2-yl)-1,1-dioxo-3,4,5,6-tetrahydro-2H-thiopyran-2-yl)acetic acid

NMR (DMSO-d₆, δ): 1.72-2.15 (4H, m), 2.40-2.50 (1H, m), 2.78-2.93 (1H, m), 3.15-3.33 (2H, m), 3.45-3.65 (2H, m), 7.09 (1H, d, J=2 Hz), 7.23 (1H, d, J=3 Hz), 7.62 (1H, d, J=3 Hz), 7.83 (2H, d, J=8 Hz), 7.93 (2H, d, J=8 Hz), 8.68 (1H, d, J=2 Hz)

MS (m/z) 416 (M−H)

### EXAMPLE 59

((2S)-2-(5-(4-(2-Methyloxazol-5-yl)phenyl)thiophen-2-yl)-1,1-dioxo-3,4,5,6-tetrahydro-2H-thiopyran-2-yl)acetic acid

NMR (DMSO-d₆, δ): 1.72-2.13 (4H, m), 2.40-2.50 (4H, m), 2.78-2.93 (1H, m), 3.15-3.30 (2H, m), 3.50-3.60 (2H, m), 7.21 (1H, d, J=3 Hz), 7.55 (1H, d, J=3 Hz), 7.59 (1H, s), 7.66-7.82 (4H, m)

MS (m/z): 430 (M−H), 432 (M+H)

### EXAMPLE 60

((2S)-2-(5-(4-(1-Methylpyrazol-5-yl)phenyl)thiophen-2-yl)-1,1-dioxo-3,4,5,6-tetrahydro-2H-thiopyran-2-yl)acetic acid

NMR (DMSO-d₆, δ): 1.72-2.14 (4H, m), 2.40-2.50 (1H, m), 2.78-2.92 (1H, m), 3.14-3.28 (2H, m), 3.48-3.67 (2H, m), 3.89 (3H, s), 6.47 (1H, d, J=2 Hz), 7.22 (1H, d, J=3 Hz), 7.48 (1H, d, J=2 Hz), 7.57 (1H, d, J=3 Hz), 7.59 (2H, d, J=8 Hz), 7.77 (2H, d, J=8 Hz)

MS (m/z) 429 (M−H), 431 (M+H)

### EXAMPLE 61

((2S)-2-(5-(4-(4-Methyloxazol-5-yl)phenyl)thiophen-2-yl)-1,1-dioxo-3,4,5,6-tetrahydro-2H-thiopyran-2-yl)acetic acid

NMR (DMSO-d₆, δ): 1.75-2.15 (4H, m), 2.40 (3H, s), 2.40-2.58 (1H, m), 2.78-2.93 (1H, m), 3.14-3.28 (2H, m), 3.50-3.67 (2H, m), 7.21 (1H, d, J=3 Hz), 7.55 (1H, d, J=3 Hz), 7.67 (2H, d, J=8 Hz), 7.78 (2H, d, J=8 Hz), 8.37 (1H, s), 12.58 (1H, br)

MS (m/z): 430 (M−H)

### EXAMPLE 62

((2S)-2-(5-(3-Methyl-4-(oxazol-5-yl)phenyl)thiophen-2-yl)-1,1-dioxo-3,4,5,6-tetrahydro-2H-thiopyran-2-yl)acetic acid

NMR (DMSO-d₆, δ): 1.70-2.13 (4H, m), 2.40-2.60 (1H, m), 2.50 (3H, s), 2.78-2.92 (1H, m), 3.15-3.28 (2H, m), 3.50-3.67 (2H, m), 7.21 (1H, d, J=3 Hz), 7.56 (2H, m), 7.58-7.68 (2H, m), 7.73 (1H, m), 8.52 (1H, s), 12.56 (1H, br)

MS (m/z): 430 (M−H), 432 (M+H)

### EXAMPLE 63

((2S)-2-(5-(4-(4-Chlorophenyl)phenyl)thiophen-2-yl)-1,1-dioxo-3,4,5,6-tetrahydro-2H-thiopyran-2-yl)acetic acid

NMR (DMSO-d₆, δ): 1.72-2.12 (4H, m), 2.43-2.55 (1H, m), 2.78-2.93 (1H, m), 3.16-3.26 (2H, m), 3.50-3.66 (2H, m), 7.21 (1H, d, J=3 Hz), 7.48-7.56 (3H, m), 7.73-7.79 (6H, m), 12.58 (1H, br)

### EXAMPLE 64

((2S)-2-(5-(4-(4-Ethylphenyl)phenyl)thiophen-2-yl)-1,1-dioxo-3,4,5,6-tetrahydro-2H-thiopyran-2-yl)acetic acid

NMR (DMSO-d₆, δ): 1.22 (3H, t, J=7 Hz), 1.72-2.15 (4H, m), 2.42-2.57 (1H, m), 2.67 (2H, q, J=7 Hz), 2.79-2.92 (1H, m), 3.16-3.27 (2H, m), 3.50-3.67 (2H, m), 7.20 (1H, d, J=3 Hz), 7.31 (2H, d, J=8 Hz), 7.52 (1H, d, J=3 Hz), 7.63 (2H, d, J=8 Hz), 7.72 (4H, s)

### EXAMPLE 65

((2S)-1,1-Dioxo-2-(5-(4-(4-trifluoromethylphenyl)-phenyl)thiophen-2-yl)-3,4,5,6-tetrahydro-2H-thiopyran-2-yl)acetic acid

NMR (DMSO-d₆, δ): 1.73-2.13 (4H, m), 2.40-2.50 (1H, m), 2.80-2.93 (1H, m), 3.15-3.27 (2H, m), 3.48-3.67 (2H, m), 7.22 (1H, d, J=3 Hz), 7.58 (1H, d, J=3 Hz), 7.78-7.93 (6H, m), 7.96 (2H, d, J=8 Hz)

### EXAMPLE 66

((2S)-2-(5-(4-(4-Methylthiophenyl)phenyl)thiophen-2-yl)-1,1-dioxo-3,4,5,6-tetrahydro-2H-thiopyran-2-yl)acetic acid

NMR (DMSO-d₆, δ): 1.73-2.15 (4H, m), 2.40-2.58 (1H, m), 2.50 (3H, s), 2.79-2.93 (1H, m), 3.15-3.28 (2H, m), 3.50-3.66 (2H, m), 7.20 (1H, d, J=3 Hz), 7.36 (2H, d, J=8 Hz), 7.53 (1H, d, J=3 Hz), 7.68 (2H, d, J=8 Hz), 7.73 (4H, s), 12.58 (1H, br)

### EXAMPLE 67

((2S)-2-(5-(4-(4-Acetylphenyl)phenyl)thiophen-2-yl)-1,1-dioxo-3,4,5,6-tetrahydro-2H-thiopyran-2-yl)acetic acid

NMR (DMSO-d₆, δ): 1.75-2.15 (4H, m), 2.45-2.60 (1H, m), 2.62 (3H, s), 2.78-2.92 (1H, m), 3.13-3.26 (2H, m), 3.48-3.65 (2H, m), 7.23 (1H, d, J=3 Hz), 7.57 (1H, d, J=3 Hz), 7.76-7.92 (6H, m), 8.05 (2H, d, J=8 Hz), 12.56 (1H, br)

MS (m/z): 467 (M−H)

### EXAMPLE 68

((2S)-2-(5-(4-(4-t-Butylphenyl)phenyl)thiophen-2-yl)-1,1-dioxo-3,4,5,6-tetrahydro-2H-thiopyran-2-yl)acetic acid

NMR (DMSO-d₆, δ): 1.32 (9H, s), 1.82-2.14 (4H, m), 2.42-2.60 (1H, m), 2.78-2.93 (1H, m), 3.13-3.26 (2H, m), 3.48-3.66 (2H, m), 7.20 (1H, d, J=3 Hz), 7.45-7.55 (3H, m), 7.63 (2H, d, J=8 Hz), 7.72 (4H, s), 12.58 (1H, br)

MS (m/z): 481 (M−H)

### EXAMPLE 69

((2S)-2-(5-(4-(4-Fluorophenyl)phenyl)thiophen-2-yl)-1,1-dioxo-3,4,5,6-tetrahydro-2H-thiopyran-2-yl)acetic acid

NMR (DMSO-d₆, δ): 1.72-2.15 (4H, m), 2.40-2.60 (1H, m), 2.80-2.93 (1H, m), 3.16-3.27 (2H, m), 3.40-3.65 (2H, m), 7.21 (1H, d, J=3 Hz), 7.31 (2H, t, J=8 Hz), 7.54 (1H, d, J=3 Hz), 7.68-7.83 (6H, m), 12.58 (1H, br)

MS (m/z) 443 (M−H)

### EXAMPLE 70

((2S)-2-(5-(4-(4-Methoxyphenyl)phenyl)thiophen-2-yl)-1,1-dioxo-3,4,5,6-tetrahydro-2H-thiopyran-2-yl)acetic acid

NMR (DMSO-d₆, δ): 1.73-2.12 (4H, m), 2.45-2.65 (1H, m), 2.78-2.93 (1H, m), 3.15-3.27 (2H, m), 3.50-3.65 (2H, m), 3.81 (3H, s), 7.04 (2H, d, J=8 Hz), 7.20 (1H, d, J=3 Hz), 7.51 (1H, d, J=3 Hz), 7.67-7.74 (6H, m), 12.56 (1H, br)

MS (m/z) 455 (M−H)

### EXAMPLE 71

((2S)-2-(5-(4-(4-Hydroxyphenyl)phenyl)thiophen-2-yl)-1,1-dioxo-3,4,5,6-tetrahydro-2H-thiopyran-2-yl)acetic acid

NMR (DMSO-d₆, δ): 1.70-2.15 (4H, m), 2.40-2.50 (1H, m), 2.79-2.93 (1H, m), 3.13-3.28 (2H, m), 3.50-3.68 (2H, m), 6.86 (2H, d, J=8 Hz), 7.19 (1H, d, J=3 Hz), 7.50 (1H, d, J=3 Hz), 7.54 (2H, d, J=8 Hz), 7.62-7.75 (4H, m), 9.60 (1H, br), 12.54 (1H, br)

MS (m/z): 441 (M−H)

### EXAMPLE 72

((2S)-1,1-Dioxo-2-(5-(4-(4-trifluoromethoxyphenyl)-phenyl)thiophen-2-yl)-3,4,5,6-tetrahydro-2H-thiopyran-2-yl)acetic acid

NMR (DMSO-d₆, δ): 1.72-2.17 (4H, m), 2.42-2.67 (1H, m), 2.79-2.93 (1H, m), 3.16-3.28 (2H, m), 3.48-3.68 (2H, m), 7.21 (1H, d, J=3 Hz), 7.47 (2H, d, J=8 Hz), 7.56 (1H, d, J=3 Hz), 7.76 (4H, s), 7.85 (2H, d, J=8 Hz), 12.58 (1H, br)

MS (m/z): 509 (M−H)

### EXAMPLE 73

((2S)-2-(5-(4-(4-Methylcarbamoylphenyl)phenyl)-thiophen-2-yl)-1,1-dioxo-3,4,5,6-tetrahydro-2H-thiopyran-2-yl) acetic acid

NMR (DMSO-d₆, δ): 1.70-2.14 (4H, m), 2.40-2.55 (1H, m), 2.81 (3H, d, J=7 Hz), 2.70-2.90 (1H, m), 3.14-3.28 (2H, m), 3.40-3.68 (2H, m), 7.22 (1H, d, J=3 Hz), 7.56 (1H, d, J=3 Hz), 7.73-7.86 (6H, m), 7.94 (2H, d, J=8 Hz), 8.52 (1H, br), 12.57 (1H, br)

MS (m/z) 484 (M+H)

### EXAMPLE 74

((2S)-2-(5-(4-(4-Dimethylcarbamoylphenyl)phenyl)-thiophen-2-yl)-1,1-dioxo-3,4,5,6-tetrahydro-2H-thiopyran-2-yl)acetic acid

NMR (DMSO-d₆, δ): 1.73-2.15 (4H, m), 2.40-2.50 (1H, m), 2.80-2.92 (1H, m), 2.93-3.10 (6H, m), 3.15-3.27 (2H, m), 3.49-3.68 (2H, m), 7.21 (1H, d, J=3 Hz), 7.50 (2H, d, J=8 Hz), 7.56 (1H, d, J=3 Hz), 7.75-7.85 (6H, m), 12.56 (1H, br)

### EXAMPLE 75

((2S)-2-(5-(4-(4-Carbamoylphenyl)phenyl)thiophen-2-yl)-1,1-dioxo-3,4,5,6-tetrahydro-2H-thiopyran-2-yl)acetic acid

NMR (DMSO-d₆, δ): 1.72-2.14 (4H, m), 2.42-2.57 (1H, m), 2.79-2.92 (1H, m), 3.15-3.28 (2H, m), 3.50-3.68 (2H, m), 7.21 (1H, d, J=3 Hz), 7.39 (1H, br), 7.55 (1H, d, J=3 Hz), 7.74-7.84 (6H, m), 7.97 (2H, d, J=8 Hz), 8.04 (1H, br), 12.57 (1H, br)

MS (m/z) 468 (M−H)

### EXAMPLE 76

((2S)-2-(5-(4-(4-Methylsulfonylaminocarbonylphenyl)-phenyl)thiophen-2-yl)-1,1-dioxo-3,4,5,6-tetrahydro-2H-thiopyran-2-yl)acetic acid

NMR (DMSO-d₆, δ): 1.74-2.15 (4H, m), 2.43-2.55 (1H, m), 2.79-2.93 (1H, m), 3.16-3.27 (2H, m), 3.40 (3H, s), 3.50-3.67 (2H, m), 7.22 (1H, d, J=3 Hz), 7.58 (1H, d, J=3 Hz), 7.76-7.94 (6H, m), 8.06 (2H, d, J=8 Hz), 12.20 (1H, br), 12.56 (1H, br)

MS (m/z): 546 (M−H)

### EXAMPLE 77

((2S)-2-(5-(4-(4-(Oxazol-5-yl)phenyl)phenyl)thiophen-2-yl)-1,1-dioxo-3,4,5,6-tetrahydro-2H-thiopyran-2-yl)acetic acid

NMR (DMSO-d₆, δ): 1.73-2.12 (4H, m), 2.43-2.57 (1H, m), 2.79-2.93 (1H, m), 3.15-3.27 (2H, m), 3.48-3.65 (2H, m), 7.22 (1H, d, J=3 Hz), 7.55 (1H, d, J=3 Hz), 7.73-7.88 (9H, m), 8.49 (1H, s)

### EXAMPLE-78

((2S)-2-(5-(4-(4-Acetamidophenyl)phenyl)thiophen-2-yl)-1,1-dioxo-3,4,5,6-tetrahydro-2H-thiopyran-2-yl)acetic acid

NMR (DMSO-d₆, δ): 1.72-2.10 (4H, m), 2.07 (3H, s), 2.40-2.50 (1H, m), 2.80-2.93 (1H, m), 3.15-3.28 (2H, m), 3.50-3.65 (2H, m), 7.21 (1H, d, J=3 Hz), 7.52 (1H, d, J=3 Hz), 7.65-7.77 (8H, m), 10.07 (1H, s), 12.57 (1H, s)

MS (m/z): 482 (M−H), 484 (M+H)

### EXAMPLE 79

((25)-2-(5-(4-(4-Dimethylaminophenyl)phenyl)thiophen-2-yl)-1,1-dioxo-3,4,5,6-tetrahydro-2H-thiopyran-2-yl)acetic acid

NMR (DMSO-d₆, δ): 1.74-2.12 (4H, m), 2.42-2.58 (1H, m), 2.78-2.90 (1H, m), 2.95 (6H, s), 3.14-3.26 (2H, m), 3.48-3.65 (2H, m), 6.80 (2H, d, J=8 Hz), 7.18 (1H, d, J=3 Hz), 7.46 (1H, d, J=3 Hz), 7.57 (2H, d, J=8 Hz), 7.66 (4H, m), 12.54 (1H, br)

MS (m/z): 470 (M+H)

### EXAMPLE 80

((2S)-2-(5-(4-(4-Ethylaminocarbonylaminophenyl)-phenyl)thiophen-2-yl)-1,1-dioxo-3,9,5,6-tetrahydro-2H-thiopyran-2-yl)acetic acid

NMR (DMSO-d₆, δ): 1.06 (3H, t, J=7 Hz), 1.75-2.15 (4H, m), 2.40-2.55 (1H, m), 2.79-2.92 (1H, m), 3.07-3.27 (4H, m), 3.50-3.60 (2H, m), 6.13 (1H, m), 7.19 (1H, d, J=3 Hz), 7.45-7.53 (3H, m), 7.60 (2H, d, J=8 Hz), 7.69 (4H, m), 8.56 (1H, s), 12.56 (1H, br)

### EXAMPLE 81

((2S)-2-(5-(4-(4-Methylsulfonylaminophenyl)phenyl)-thiophen-2-yl)-1,1-dioxo-3,4,5,6-tetrahydro-2H-thiopyran-2-yl)acetic acid

NMR (DMSO-d₆, δ): 1.74-2.15 (4H, m), 2.42-2.57 (1H, m), 2.78-2.93 (1H, m), 3.03 (3H, s), 3.16-3.28 (2H, m), 3.52-3.67 (2H, m), 7.21 (1H, d, J=3 Hz), 7.30 (2H, d, J=8 Hz), 7.52 (1H, d, J=3 Hz), 7.68-7.78 (6H, m), 9.90 (1H, s), 12.56 (1H, br)

### EXAMPLE 82

((2S)-2-(5-(4-(4-Methoxycarbonylaminophenyl)phenyl)-thiophen-2-yl)-1,1-dioxo-3,4,5,6-tetrahydro-2H-thiopyran-2-yl)acetic acid

NMR (DMSO-d₆, δ): 1.73-2.14 (4H, m), 2.42-2.58 (1H, m), 2.79-2.93 (1H, m), 3.13-3.27 (2H, m), 3.45-3.65 (2H, m), 3.69 (3H, s), 7.19 (1H, d, J=3 Hz), 7.51 (1H, d, J=3 Hz), 7.55 (2H, d, J=8 Hz), 7.67 (2H, d, J=8 Hz), 7.70 (4H, s), 9.78 (1H, s), 12.55 (1H, br)

MS (m/z): 499 (M−H)

### EXAMPLE 83

((2S)-2-(5-(4-(4-Methylphenyl)phenyl)thiophen-2-yl)-1,1-dioxo-3,4,5,6-tetrahydro-2H-thiopyran-2-yl)acetic acid

NMR (DMSO-d₆, δ): 1.73-2.12 (4H, m), 2.35 (3H, s), 2.40-2.54 (1H, m), 2.78-2.89 (1H, m), 3.14-3.23 (2H, m), 3.46-3.63 (2H, m), 7.20 (1H, d, J=3 Hz), 7.27 (2H, d, J=8 Hz), 7.52 (1H, d, J=3 Hz), 7.59 (2H, d, J=8 Hz), 7.70 (4H, m)

### EXAMPLE 84

((2S)-2-(5-(4-(3-Cyanophenyl)phenyl)thiophen-2-yl)-1,1-dioxo-3,4,5,6-tetrahydro-2H-thiopyran-2-yl)acetic acid

NMR (DMSO-d₆, δ): 1.74-2.15 (4H, m), 2.42-2.50 (1H, m), 2.79-2.95 (1H, m), 3.15-3.28 (2H, m), 3.50-3.70 (2H, m), 7.22 (1H, m), 7.57 (1H, m), 7.64-7.76 (1H, m), 7.76-7.88 (5H, m), 8.06-8.15 (1H, m), 8.23 (1H, br), 12.56 (1H, br)

### EXAMPLE 85

((2S)-2-(5-(4-(3-Methylphenyl)phenyl)thiophen-2-yl)-1,1-dioxo-3,4,5,6-tetrahydro-2H-thiopyran-2-yl)acetic acid

NMR (DMSO-d₆, δ): 1.73-2.12 (4H, m), 2.39 (3H, s), 2.42-2.53 (1H, m), 2.78-2.88 (1H, m), 3.13-3.24 (2H, m), 3.47-3.62 (2H, m), 7.18 (2H, m), 7.33 (1H, t, J=8 Hz), 7.46-7.53 (3H, m), 7.73 (4H, s)

### EXAMPLE 86

((2S)-2-(5-(4-(2-Methylphenyl)phenyl)thiophen-2-yl)-1,1-dioxo-3,4,5,6-tetrahydro-2H-thiopyran-2-yl)acetic acid

NMR (DMSO-d₆, δ): 1.72-2.15 (4H, m), 2.27 (3H, s), 2.42-2.58 (1H, m), 2.83-2.94 (1H, m), 3.15-3.28 (2H, m), 3.47-3.67 (2H, m), 7.19 (1H, d, J=3 Hz), 7.20-7.34 (4H, m), 7.40 (2H, d, J=8 Hz), 7.53 (1H, d, J=3 Hz), 7.71 (2H, d, J=8 Hz)

MS (m/z): 441 (M+H)

### EXAMPLE 87

((2S)-1,1-Dioxo-2-(5-(4-(2-phenyl-2H-tetrazol-5-yl)phenyl)thiophen-2-yl)-3,4,5,6-tetrahydro-2H-thiopyran-2-yl)acetic acid

NMR (DMSO-d₆, δ): 1.74-2.14 (4H, m), 2.43-2.58 (1H, m), 2.81-2.93 (1H, m), 3.16-3.28 (2H, m), 3.50-3.67 (2H, m), 7.25 (1H, d, J=3 Hz), 7.62-7.76 (4H, m), 7.93 (2H, d, J=8 Hz), 8.15-8.27 (4H, m), 12.58 (1H, br)

MS (m/z): 493 (M−H)

### EXAMPLE 88

((2S)-2-(5-(4-((E)-2-(Oxazol-5-yl)ethenyl)phenyl)-thiophen-2-yl)-1,1-dioxo-3,4,5,6-tetrahydro-2H-thiopyran-2-yl)acetic acid

NMR (DMSO-d₆, δ): 1.75-2.15 (4H, m), 2.40-2.60 (1H, m), 2.79-2.93 (1H, m), 3.14-3.26 (2H, m), 3.47-3.66 (2H, m), 7.06-7.14 (1H, d, J=15 Hz), 7.18 (1H, d, J=3 Hz), 7.25-7.33 (1H, d, J=15 Hz), 7.29 (1H, s), 7.53 (1H, d, J=3 Hz), 7.63-7.72 (4H, m), 8.40 (1H, s), 12.58 (1H, br)

MS (m/z) 442 (M−H)

### EXAMPLE 89

((2S)-2-(5-(4-(Oxazol-5-ylmethoxy)phenyl)thiophen-2-yl)-1,1-dioxo-3,4,5,6-tetrahydro-2H-thiopyran-2-yl)acetic acid

NMR (DMSO-d₆, δ): 1.72-2.12 (4H, m), 2.38-2.54 (1H, m), 2.76-2.90 (1H, m), 3.12-3.26 (2H, m), 3.45-3.62 (2H, m), 5.22 (2H, s), 7.08 (2H, d, J=8 Hz), 7.14 (1H, d, J=3 Hz), 7.36 (2H, m), 7.59 (2H, d, J=8 Hz), 8.43 (1H, s)

MS (m/z) 446 (M−H)

### EXAMPLE 90

((2S)-2-(5-(4-((E)-2-(Methylcarbamoyl)ethenyl)phenyl)-thiophen-2-yl)-1,1-dioxo-3,4,5,6-tetrahydro-2H-thiopyran-2-yl)acetic acid

NMR (DMSO-d₆, δ): 1.72-2.14 (4H, m), 2.40-2.53 (1H, m), 2.71 (3H, d, J=7 Hz), 2.79-2.93 (1H, m), 3.14-3.27 (2H, m), 3.50-3.66 (2H, m), 6.64 (1H, d, J=15 Hz), 7.21 (1H, d, J=3 Hz), 7.43 (1H, d, J=15 Hz), 7.54 (1H, d, J=3 Hz), 7.63 (2H, d, J=8 Hz), 7.70 (2H, d, J=8 Hz), 8.08 (1H, m), 12.56 (1H, br)

MS (m/z) 432 (M−H)

### EXAMPLE 91

((2S)-2-(5-(4-(Oxazol-5-ylcarbonylamino)phenyl)-thiophen-2-yl)-1,1-dioxo-3,4,5,6-tetrahydro-2H-thiopyran-2-yl)acetic acid

NMR (DMSO-d₆, δ): 1.72-2.13 (4H, m), 2.41-2.53 (1H, m), 2.77-2.93 (1H, m), 3.13-3.27 (2H, m), 3.48-3.65 (2H, m), 7.18 (1H, d, J=3 Hz), 7.43 (1H, d, J=3 Hz), 7.66 (2H, d, J=8 Hz), 7.80 (2H, d, J=8 Hz), 8.01 (1H, s), 8.68 (1H, s), 10.55 (1H, s), 12.55 (1H, br)

MS (m/z) 459 (M−H)

### EXAMPLE 92

((2S)-1,1-Dioxo-2-{5-[4-(oxazol-4-yl)phenyl]thiophen-2-yl}-3,4,5,6-tetrahydro-2H-thiopyran-2-yl)acetic acid

NMR (DMSO-d₆, δ): 1.80-2.20 (4H, m), 2.50-2.60 (1H, m), 2.90-3.00 (1H, m), 3.28 (1H, d, J=15 Hz), 3.30-3.70 (2H, m), 3.67 (1H, d, J=15 Hz), 7.30 (1H, d, J=3 Hz), 7.62 (1H, d, J=2 Hz), 7.82 (2H, d, J=9 Hz), 7.94 (2H, d, J=9 Hz), 8.69 (1H, s), 8.79 (1H, s)

### EXAMPLE 93

((2S)-1,1-Dioxido-2-{5-[4-(thiazol-4-yl)phenyl]-thiophen-2-yl}tetrahydro-2H-thiopyran-2-yl)acetic acid

NMR (DMSO-d₆, δ): 1.80-2.15 (4H, m), 2.50-2.60 (1H, m), 2.90-3.00 (1H, m), 3.27 (1H, d, J=15 Hz), 3.60-3.70 (2H, m), 3.66 (1H, d, J=15 Hz), 7.28 (1H, d, J=5 Hz), 7.62 (1H, d, J=5 Hz), 7.82 (2H, d, J=8 Hz), 8.12 (2H, d, J=8 Hz), 8.32 (1H, d, J=2 Hz), 9.29 (1H, d, J=2 Hz)

### EXAMPLE 94

4-Methoxybenzyl ((2S)-2-(5-(4-butoxyphenyl)thiophen-2-yl]-1,1-dioxo-3,4,5,6-tetrahydro-2H-thiopyran-2-yl)acetate (150 mg) was dissolved in ethyl acetate (2 ml) and 4N hydrogenchloride in ethyl acetate (2 ml) was added. After stirring at 20° C. overnight, the mixture was partitioned between ethyl acetate and water. The organic layer was separated, washed with water and brine, dried over magnesium sulfate, and evaporated. The residue was purified by preparative thin-layer chromatography (hexane/EtOAc=1:2) and triturated with hexane to give {(2S)-2-[5-(4-butoxyphenyl)thiophen-2-yl]-1,1-dioxo-3,4,5,6-tetrahydro-2H-thiopyran-2-yl} acetic acid (82 mg) as colorless crystals.

mp: 179-180° C.

NMR (CDCl₃, δ): 0.98 (3H, t, J=7 Hz), 1.54 (2H, sxtet, J=7 Hz), 1.70-2.20 (6H, m), 2.65-2.90 (2H, m), 3.00-3.20 (3H, m), 3.45 (1H, d, J=15 Hz), 3.98 (2H, t, J=7 Hz), 6.88 (2H, d, J=9 Hz), 7.10 (1H, d, J=4 Hz), 7.19 (1H, d, J=4 Hz), 7.49 (2H, d, J=9 Hz)

The following compounds were obtained in a similar manner to that of Example 94.

### EXAMPLE 95

{(2S)-1,1-Dioxo-2-[5-(4-pentyloxyphenyl)thiophen-2-yl]-3,4,5,6-tetrahydro-2H-thiopyran-2-yl}acetic acid

mp: 183-185° C.

NMR (CDCl₃, δ): 0.94 (3H, t, J=7 Hz), 1.35-1.50 (4H, m), 1.70-2.20 (6H, m), 2.65-2.90 (2H, m), 3.00-3.20 (2H, m), 3.16 (1H, d, J=15 Hz), 3.46 (1H, d, J=15 Hz), 3.97 (2H, t, J=7 Hz), 6.88 (2H, d, J=9 Hz), 7.10 (1H, d, J=4 Hz), 7.19 (1H, d, J=4 Hz), 7.49 (2H, d, J=9 Hz)

### EXAMPLE 96

((2S)-1,1-Dioxo-2-{5-[4-(1-methylethoxy)phenyl]-thiophen-2-yl}-3, 4,5,6-tetrahydro-2H-thiopyran-2-yl) acetic acid

mp 184-185° C.

NMR (CDCl₃, δ): 1.35 (6H, d, J=7 Hz), 1.75-2.00 (4H, m), 2.05-2.20 (2H, m), 2.65-2.90 (2H, m), 3.00-3.15 (2H, m), 3.16 (1H, d, J=15 Hz), 3.46 (1H, d, J=15 Hz), 4.57 (1H, septet, J=7 Hz), 6.87 (2H, d, J=9 Hz), 7.10 (1H, d, J=4 Hz), 7.19 (1H, d, J=4 Hz), 7.48 (2H, d, J=9 Hz)

### EXAMPLE 97

{(2S)-1,1-Dioxo-2-[5-(6-fluorobenzothiophen-2-yl)thiophen-2-yl]-3,4,5,6-tetrahydro-2H-thiopyran-2-yl}acetic acid

NMR (DMSO-d₆, δ): 1.70-2.10 (4H, m), 2.40-2.50 (1H, m), 2.82 (1H, d, J=10 Hz), 3.10-3.30 (2H, m), 3.50-3.65 (2H, m), 7.20 (1H, d, J=4 Hz), 7.28 (1H, dt, J=2, 9 Hz), 7.39 (1H, d, J=4 Hz), 7.68 (1H, s), 7.85 (1H, dd, J=5, 9 Hz), 7.91 (2H, dd, J=2, 9 Hz)

### EXAMPLE 98

A solution of 4-methoxybenzyl {(2S)-1,1-dioxo-2-[5-(6-methoxymethoxynaphthalen-2-yl)thiophen-2-yl]-3,4,5,6-tetrahydro-2H-thiopyran-2-yl}acetate (216 mg) in methanol (5 ml) was hydrogenated over palladium hydroxide(100 mg) at 3 atm at room temperature for 8 hours. The catalyst was filtered off through celite and the filtrate was concentrated in vacuo. The residue was purified by flash column chromatography on silica gel eluting with a mixture of methanol and chloroform (1:30) to give {(2S)-1,1-dioxo-2-[5-(6-methoxymethoxynaphthalen-2-yl)thiophen-2-yl]-3,4,5,6-tetrahydro-2H-thiopyran-2-yl}acetic acid (147 mg) as a white amorphous.

NMR (DMSO-d₆, δ): 1.74-2.10 (4H, m), 2.72-2.90 (2H, m), 3.13-3.24 (2H, m), 3.43 (2H, s), 3.58 (2H, d, J=15 Hz), 5.33 (2H, s), 7.21 (1H, d, J=4 Hz), 7.27 (1H, dd, J=8, 4 Hz), 7.44 (1H, d, J=2 Hz), 7.56 (1H, d, J=4 Hz), 7.77-7.88 (2H, m), 7.94 (1H, d, J=8 Hz), 8.14 (1H, s)

MS (m/z): 459 (M⁺−H), 45 (bp)

The following compounds were obtained in a similar manner to that of Preparation 94.

### EXAMPLE 99

((2S)-2-(5-(4-(4-Aminophenyl)phenyl)thiophen-2-yl)-1,1-dioxo-3,4,5,6-tetrahydro-2H-thiopyran-2-yl)acetic acid hydrochloride

NMR (DMSO-d₆, δ): 1.70-2.15 (4H, m), 2.40-2.60 (1H, m), 2.79-2.93 (1H, m), 3.15-3.27 (2H, m), 3.48-3.67 (2H, m), 6.64 (2H, d, J=8 Hz), 7.18 (1H, d, J=3 Hz), 7.41 (2H, d, J=8 Hz), 7.46 (1H, d, J=3 Hz), 7.57-7.69 (4H, m)

### EXAMPLE 100

((2S)-2-(5-(4-(4-Aminophenyl)phenyl)thiophen-2-yl)-1,1-dioxo-3,4,5,6-tetrahydro-2H-thiopyran-2-yl)acetic acid hydrochloride

NMR (DMSO-d₆, δ): 1.72-2.12 (4H, m), 2.40-2.56 (1H, m), 2.79-2.91 (1H, m), 3.14-3.27 (2H, m), 3.48-3.65 (2H, m), 7.15-7.26 (3H, m), 7.53 (1H, d, J=3 Hz), 7.67-7.78 (6H, m)

MS (m/z) 440 (M−H)

### EXAMPLE 101

To a solution of benzothiophene-5-boronic acid pinacol ester (574 mg) in 1,4-dioxane (10 ml) were added 2-(trimethylsilyl)ethyl [(2S)-2-(5-bromothiophen-2-yl)-1,1-dioxo-3,4,5,6-tetrahydro-2H-thiopyran-2-yl]acetate (500 mg), dichlorobis(triphenylphosphine)palladium(II) (23.2 mg), and 2M aqueous sodium carbonate (2.2 ml). After stirring at 80° C. for 8 hours, the mixture was partitioned between ethyl acetate and water. The organic layer was separated, washed with water and brine, dried over magnesium sulfate, and evaporated in vacuo. The residue was chromatographed on silica gel eluting with a mixture of ethyl acetate and hexane (1:2) to give 2-(trimethyl)silylethyl {(2S)-2-[5-(benzothiophen-5-yl)thiophen-2-yl]-1,1-dioxo-3,4,5,6-tetrahydro-2H-thiopyran-2-yl}acetate (343 mg) as a white powder.

NMR (CDCl₃, δ): 0.05 (9H, s), 0.81 (2H, t, J=7 Hz), 1.78-2.06 (2H, m), 2.10-2.21 (2H, m), 2.71-2.89 (2H, m), 3.06-3.16 (3H, m), 3.42 (1H, d, J=15 Hz), 3.99-4.07 (2H, m), 7.27-7.29 (2H, m), 7.33 (1H, d, J=7 Hz), 7.47 (1H, d, J=7 Hz), 7.58 (1H, dd, J=7.5, 1 Hz), 7.85 (1H, d, J=7.5 Hz), 8.03 (1H, d, J=2 Hz)

The following compounds were obtained in a similar manner to that of Example 101.

### EXAMPLE 102

2-(Trimethyl)silylethyl {(2S)-1,1-dioxo-2-[5-(6-methoxycarbonylnaphthalen-2-yl)thiophen-2-yl]-3,4,5,6-tetrahydro-2H-thiopyran-2-yl}acetate

NMR (CDCl₃, δ): 0.05 (9H, s), 0.87 (2H, t, J=7 Hz), 1.82-2.10 (2H, m), 2.15-2.30 (2H, m), 2.77-2.99 (2H, m), 3.14-3.24 (3H, m), 3.50 (1H, d, J=15 Hz), 4.04 (3H, s), 4.05-4.12 (2H, m), 7.37 (1H, d, J=4 Hz), 7.48 (1H, d, J=4 Hz), 7.83 (1H, dd, J=8, 1 Hz), 7.95 (2H, AB, J=8, 7.5 Hz), 8.10-8.14 (2H, m), 8.63 (1H, s)

### EXAMPLE 103

2-(Trimethyl)silylethyl {(2S)-1,1-dioxo-2-[5-(6-methoxynaphthalen-2-yl)thiophen-2-yl]-3,4,5,6-tetrahydro-2H-thiopyran-2-yl}acetate

NMR (CDCl₃, δ): 0.05(9H, s), 0.82 (2H, t, J=7 Hz), 1.77-2.07 (2H, m), 2.10-2.24 (2H, m), 2.72-3.95 (2H, m), 3.08-3.18 (3H, m), 3.44 (1H, d, J=15 Hz), 3.93 (3H, s), 3.99-4.06 (2H, m), 7.10-7.17 (2H, m), 7.27 (1H, d, J=4 Hz), 7.32 (1H, d, J=4 Hz), 7.65-7.74 (3H, m), 7.97 (1H, s)

MS (m/z) 532 (M⁺+H), 79(bp)

### EXAMPLE 104

2-(Trimethyl)silylethyl {(2S)-1,1-dioxo-2-[5-(6-formylnaphthalen-2-yl)thiophen-2-yl]-3,4,5,6-tetrahydro-2H-thiopyran-2-yl}acetate

NMR (CDCl₃, δ): 0.05 (9H, s), 0.83 (2H, t, J=7 Hz), 1.83-2.05 (2H, m), 2.11-2.25 (2H, m), 3.10-3.19 (3H, m), 2.77-2.89 (2H, m), 3.46 (1H, d, J=15 Hz), 4.00-4.08 (2H, m), 7.32 (1H, d, J=2 Hz), 7.45 (1H, d, J=2 Hz), 7.81-8.02 (4H, m), 8.10 (1H, s), 8.31 (1H, s), 10.15 (1H, s)

### EXAMPLE 105

2-(Trimethyl)silylethyl ((2S)-1,1-dioxo-2-{5-[6-(oxazol-5-yl)naphthalen-2-yl]thiophen-2-yl]-3,4,5,6-tetrahydro-2H-thiopyran-2-yl} acetate

NMR (CDCl₃, δ): 0.05 (9H, s), 0.87 (2H, t, J=7 Hz), 1.83-2.07 (2H, m), 2.15-2.28 (2H, m), 2.78-3.00 (2H, m), 3.13-3.23 (3H, m), 3.49 (1H, d, J=15 Hz), 4.05-4.13 (2H, m), 7.10-7.15 (2H, m), 7.30-7.35 (2H, m), 7.67 (2H, s), 7.74 (1H, d, J=7.5 Hz), 7.95 (1H, s)

MS (m/z): 529 (M⁺+H), 115 (bp)

### EXAMPLE 106

2-(Trimethyl)silylethyl {(2S)-1,1-dioxo-2-[5-(6-hydroxynaphthalen-2-yl)thiophen-2-yl]-3,4,5,6-tetrahydro-2H-thiopyran-2-yl}acetate

NMR (CDCl₃, δ) 0.05 (9H, s), 0.88 (2H, t, J=7 Hz), 1.83-2.09 (2H, m), 2.15-2.29 (2H, m), 2.77-3.00 (2H, m), 3.10-3.28 (3H, m), 3.49 (1H, d, J=15 Hz), 4.05-4.13 (2H, m), 7.10-7.14 (2H, m), 7.30-7.34 (2H, m), 7.65 (2H, s), 7.74 (1H, d, J=7.5 Hz), 7.95 (1H, s)

MS (m/z): 515 (M⁺−H), 115 (bp)

### EXAMPLE 107

2-(Trimethyl)silylethyl {(2S)-2-[5-(benzothiophen-3-yl)thiophen-2-yl]-1,1-dioxo-3,4,5,6-tetrahydro-2H-thiopyran-2-yl}acetate

NMR (CDCl₃, δ): 0.81 (2H, t, J=7 Hz), 1.79-2.04 (2H, m), 2.11-2.23 (2H, m), 2.73-2.93 (2H, m), 3.05-3.18 (3H, m), 3.47 (1H, d, J=15 Hz), 4.03 (2H, dd, J=8, 7.5 Hz), 7.28-7.34 (2H, m), 7.36-7.46 (2H, m), 7.53 (1H, s), 7.89 (1H, dd, J=7, 2 Hz), 8.12 (1H, dd, J=7, 2 Hz)

### EXAMPLE 108

2-(Trimethyl)silylethyl {(2S)-2-[5-(6-cyanonaphthalen-2-yl)thiophen-2-yl]-1,1-dioxo-3,4,5,6-tetrahydro-2H-thiopyran-2-yl}acetate

NMR (CDCl₃, δ): 0.05 (9H, s), 0.87 (2H, t, J=7 Hz), 1.84-2.10 (2H, m), 2.17-2.30 (2H, m), 2.80-3.93 (2H, m), 3.16-3.25 (3H, m), 3.50 (1H, d, J=15 Hz), 4.05-4.14 (2H, m), 7.37 (1H, d, J=2 Hz), 7.50 (1H, d, J=2 Hz), 7.68 (1H, dd, J=8, 1 Hz), 7.87-7.97 (2H, m), 8.13 (1H, s), 8.25 (1H, s)

### EXAMPLE 109

2-(Trimethyl)silylethyl {(2S)-1,1-dioxo-2-[5-(6-ethoxynaphthalen-2-yl)thiophen-2-yl]-3,4,5,6-tetrahydrd-2H-thiopyran-2-yl}acetate

NMR (CDCl₃, δ): 0.05 (9H, s), 0.87 (2H, t, J=7 Hz), 1.55 (3H, t, J=7 Hz), 1.80-2.09 (2H, m), 2.14-2.28 (2H, m), 2.75-3.01 (2H, m), 3.09-3.24 (3H, m), 3.49 (1H, d, J=15 Hz), 4.04-4.12 (2H, m), 4.21 (2H, q, J=7 Hz), 7.15 (1H, s), 7.20 (1H, dd, J=7.5, 1 Hz), 7.24-7.39 (2H, m), 7.72-7.80 (3H, m), 8.05 (1H, s)

### EXAMPLE 110

2-(Trimethyl)silylethyl {(2S)-1,1-dioxo-2-[5-(1-methyl-2,3-dihydro-2-oxoindol-5-yl)thiophen-2-yl]-3,4,5,6-tetrahydro-2H-thiopyran-2-yl}acetate

NMR (CDCl₃, δ): 0.03 (9H, s), 0.83 (2H, t, J=7 Hz), 1.80-2.06 (2H, m), 2.11-2.22 (2H, m), 2.76-2.83 (2H, m), 3.08-3.15 (3H, m), 3.23 (3H, s), 3.42 (1H, d, J=15 Hz), 3.54 (2H, s), 4.00-4.07 (2H, m), 6.80 (1H, d, J=8 Hz), 7.15 (1H, d, J=4 Hz), 7.23 (1H, d, J=4 Hz), 7.48 (1H, s), 7.52 (1H, d, J=8 Hz)

### EXAMPLE 111

2-(Trimethyl)silylethyl {(2S)-2-[5-(5-methylbenzothiophen-2-yl)thiophen-2-yl]-1,1-dioxo-3,4,5,6-tetrahydro-2H-thiopyran-2-yl}acetate

NMR (CDCl₃, δ): 0.04 (9H, s), 0.85 (2H, t, J=7 Hz), 1.78-2.06 (2H, m), 2.12-2.26 (2H, m), 2.49 (3H, s), 2.70-2.95 (2H, m), 3.08-3.20 (3H, m), 3.44 (1H, d, J=15 Hz), 4.03-4.11 (2H, m), 7.18 (1H, dd, J=8, 1 Hz), 7.24 (2H, s), 7.37 (1H, s), 7.55 (1H, s), 7.69 (1H, d, J=8 Hz)

### EXAMPLE 112

2-(Trimethyl)silylethyl {(2S)-2-[5-(5-methoxybenzothiophen-2-yl)thiophen-2-yl]-1,1-dioxo-3,4,5,6-tetrahydro-2H-thiopyran-2-yl}acetate

NMR (CDCl₃, δ): 0.03 (9H, s), 0.87 (2H, t, J=7 Hz), 1.80-2.08 (2H, m), 2.12-2.27 (2H, m), 2.73-2.95 (2H, m), 3.10-3.24 (3H, m), 3.44 (1H, d, J=15 Hz), 3.91 (3H, s), 4.04-4.10 (2H, m), 6.99 (2H, dd, J=8, 1 Hz), 7.20-7.25 (3H, m), 7.36 (1H, s), 7.67 (1H, d, J=8 Hz)

### EXAMPLE 113

2-(Trimethyl)silylethyl {(2S)-2-[5-(6-methoxybenzothiophen-2-yl)thiophen-2-yl]-1,1-dioxo-3,4,5,6-tetrahydro-2H-thiopyran-2-yl}acetate

NMR (CDCl₃, δ): 0.04 (9H, s), 0.85 (2H, t, J=7 Hz), 1.84-2.05 (2H, m), 2.12-2.24 (2H, m), 2.73-2.94 (2H, m), 3.10-3.18 (3H, m), 3.43 (1H, d, J=15 Hz), 3.92 (3H, s), 4.04-4.10 (2H, m), 7.00 (1H, dd, J=7.5, 2 Hz), 7.22 (2H, AB, J=8, 3 Hz), 7.27-3.01 (1H, m), 7.35 (1H, s), 7.64 (1H, d, J=8 Hz)

### EXAMPLE 114

4-Methoxybenzyl {(2S)-1,1-dioxo-2-[5—(6-methoxymethoxynaphthalen-2-yl)thiophen-2-yl]-3,4,5,6-tetrahydro-2H-thiopyran-2-yl)acetate

NMR (CDCl₃, δ): 0.05 (9H, s), 1.78-2.03 (2H, m), 2.09-2.20 (2H, m), 2.65-2.90 (2H, m), 3.05-3.19 (3H, m), 3.48 (1H, d, J=15 Hz), 3.54 (3H, s), 3.59 (3H, s), 4.90 (2H, AB, J=8, 8 Hz), 6.68 (2H, d, J=8 Hz), 7.04 (2H, d, J=8 Hz), 7.19-7.27 (3H, m), 7.39 (1H, d, J=2 Hz), 7.64-7.79 (3H, m), 7.96 (1H, s)

### EXAMPLE-115

2-(Trimethyl)silylethyl {(2S)-2-[5-(5-fluorobenzothiophen-2-yl)thiophen-2-yl]-1,1-dioxo-3,4,5,6-tetrahydro-2H-thiopyran-2-yl)acetate

NMR (CDCl₃, δ): 0.03 (9H, s), 0.85 (2H, t, J=7 Hz), 1.82-2.08 (2H, m), 2.11-2.28 (2H, m), 2.74-2.94 (2H, m), 3.10-3.20 (3H, m), 3.44 (1H, d, J=15 Hz), 4.04-4.10 (2H, m), 7.10 (1H, ddd, J=8, 8, 2 Hz), 7.25(1H, s), 7.39(1H, s), 7.41(1H, dd, J=8, 2 Hz), 7.73(1H, dd, J=8, 4 Hz)

### EXAMPLE 116

2-(Trimethyl)silylethyl [(2S)-1,1-dioxo-2-(5-{6-[methylcarbamoyl]-2-naphthalen-2-yl}thiophen-2-yl)-3,4,5,6-tetrahydro-2H-thiopyran-2-yl]acetate

NMR (CDCl₃, δ): 0.05 (9H, s), 0.82 (2H, t, J=7 Hz), 1.80-2.05 (2H, m), 2.13-2.24 (2H, m), 2.74-2.90 (2H, m), 3.04-3.18 (3H, m), 3.09 (3H, d, J=7 Hz), 3.45 (1H, d, J=15 Hz), 4.00-4.07 (2H, m), 6.24-6.29 (1H, m), 7.29 (1H, d, J=2 Hz), 7.40 (1H, d, J=2 Hz), 7.75-7.92 (4H, m), 8.05 (1H, s), 8.24 (1H, s)

MS (m/z): 556 (M⁺−H), 153 (bp)

### EXAMPLE 117

[(2S)-1,1-Dioxo-2-(5-{4′-(methoxycarbonyl)-4-biphenylyl)thiophen-2-yl)-3,4,5,6-tetrahydro-2H-thiopyran-2-yl]acetate

mp: 258-259° C. (dec.)

NMR (CDCl₃, δ): 1.60-2.00 (2H, m), 2.10-2.22 (2H, m), 2.72-2.89 (2H, m), 3.04-3.17 (2H, m), 3.21 (1H, d, J=15 Hz), 3.47 (1H, d, J=15 Hz), 3.95 (3H, s), 7.27-7.33 (2H, m), 7.60-7.69 (6H, m), 8.11(2H, d, J=8 Hz)

MS (m/z): (M⁺−H), 174 (bp)

### EXAMPLE 118

[(2S)-1,1-Dioxo-2-(5-{4′-[methylaminosulfonyl]-4-biphenylyl]-4-yl)thiophen-2-yl)-3,4,5,6-tetrahydro-2H-thiopyran-2-yl]acetic acid

NMR (CDCl₃, δ): 1.83-2.05 (3H, m), 2.13-2.24 (2H, m), 2.80-2.88 (2H, m), 3.13-3.25 (2H, m), 3.50 (1H, d, J=15 Hz), 3.69 (3H, s), 7.29 (1H, d, J=4 Hz), 7.34 (1H, d, J=4 Hz), 7.60 (2H, d, J=7.5 Hz), 7.70 (2H, d, J=7.5 Hz), 7.74 (2H, d, J=8 Hz), 7.92 (2H, d, J=8 Hz)

MS (m/z): 518 (M⁺−H), 127 (bp)

### EXAMPLE 119

2-(Trimethylsilyl)ethyl ((2S)-1,1-dioxo-2-(5-(4-(pyrazin-2-yl)phenyl)thiophen-2-yl)-3,4,5,6-tetrahydro-2H-thiopyran-2-yl)acetate

NMR (CDCl₃, δ-0.04 (9H, s), 0.78-0.91 (2H, m), 1.80-2.30 (4H, m), 2.73-2.94 (2H, m), 3.04-3.25 (2H, m), 3.14 (1H, d, J=15 Hz), 3.44 (1H, d, J=15 Hz), 3.98-4.10 (2H, m), 7.28 (1H, d, J=3 Hz), 7.35 (1H, d, J=3 Hz), 7.74 (2H, d, J=8 Hz), 8.04 (2H, d, J=8 Hz), 8.51 (1H, d, J=2 Hz), 8.63 (1H, d, J=2 Hz), 9.05 (1H, s)

### EXAMPLE 120

2-(Trimethylsilyl)ethyl ((2S)-1,1-dioxo-2-(5-(4-(pyrimidin-5-yl)phenyl)thiophen-2-yl)-3,4,5,6-tetrahydro-2H-thiopyran-2-yl)acetate

NMR (CDCl₃, δ-0.03 (9H, s), 0.78-0.88 (2H, m), 1.79-2.26 (4H, m), 2.72-2.91 (2H, m), 3.02-3.24 (2H, m), 3.14 (1H, d, J=15 Hz), 3.44 (1H, d, J=15 Hz), 3.99-4.11 (2H, m), 7.31 (1H, d, J=3 Hz), 7.37 (1H, d, J=3 Hz), 7.63 (2H, d, J=BHz), 7.77 (2H, d, J-8 Hz), 8.98 (2H, s), 9.20 (1H, s)

MS (m/z) 529 (M+H)

### EXAMPLE 121

2-(Trimethylsilyl)ethyl ((2S)-2-(5-(4-(2-methylthiazol-4-yl)phenyl)thiophen-2-yl)-1,1-dioxo-3,4,5,6-tetrahydro-2H-thiopyran-2-yl)acetate

NMR (CDCl₃, δ-0.04 (9H, s), 0.79-0.91 (2H, m), 1.79-2.06 (2H, m), 2.11-2.29 (2H, m), 2.72-3.04 (2H, m), 2.79 (3H, s), 3.02-3.24 (2H, m), 3.14 (1H, d, J=15 Hz), 3.44 (1H, d, J=15 Hz), 3.99-4.10 (2H, m), 7.24 (1H, d, J=3 Hz), 7.30 (1H, d, J=3 Hz), 7.34 (1H, s), 7.64 (2H, d, J=8 Hz), 7.88 (2H, d, J=8 Hz)

MS (m/z) 548 (M+H)

### EXAMPLE 122

2-(Trimethylsilyl)ethyl ((2S)-2-(5-(4-(isoxazol-5-yl)phenyl)thiophen-2-yl)-1,1-dioxo-3,4,5,6-tetrahydro-2H-thiopyran-2-yl)acetate

NMR (CDCl₃, δ): -0.04 (9H, s), 0.79-0.89 (2H, m), 1.80-2.09 (2H, m), 2.09-2.28 (2H, m), 2.73-2.94 (2H, m), 3.05-3.24 (2H, m), 3.15 (1H, d, J=15 Hz), 3.45 (1H, d, J=15 Hz), 4.00-4.12 (2H, m), 6.54 (1H, d, J=2 Hz), 7.27 (1H, d, J=3 Hz), 7.34 (1H, d, J=3 Hz), 7.69 (2H, d, J=8 Hz), 7.79 (2H, d, J=8 Hz), 8.30 (1H, d, J=2 Hz)

### EXAMPLE 123

2-(Trimethylsilyl)ethyl ((2S)-2-(5-(4-(2-methyloxazol--yl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.88 (2H, m), 1.79-2.26 (4H, m), 2.54 (3H, s), 2.72-2.91 (2H, m), 3.02-3.24 (2H, m), 3.14 (1H, d, J=15 Hz), 3.44 (1H, d, J=15 Hz), 3.99-4.11 (2H, m), 7.29 (3H, m), 7.61 (4H, m)

MS (m/z): 532 (M+H)

### EXAMPLE-124

2-(Trimethylsilyl)ethyl ((2S)-2-(5-(4-(1-methylpyrazol-5-yl)phenyl)thiophen-2-yl)-1,1-dioxo-3,4,5,6-tetrahydro-2H-thiopyran-2-yl)acetate

NMR (CDCl₃, δ): -0.04 (9H, s), 0.78-0.91 (2H, m), 1.78-2.29 (4H, m), 2.75-2.94 (2H, m), 3.06-3.21 (2H, m), 3.15 (1H, d, J=15 Hz), 3.43 (1H, d, J=15 Hz), 3.92 (3H, s), 3.96-4.10 (2H, m), 6.33 (1H, d, J=2 Hz), 7.28 (1H, d, J=3 Hz), 7.31 (1H, d, J=3 Hz), 7.42 (2H, d, J=8 Hz), 7.52 (1H, d, J=2 Hz), 7.67 (2H, d, J=8 Hz)

### EXAMPLE 125

2-(Trimethylsilyl)ethyl ((2S)-2-(5-(4-(4-methyloxazol-5-yl)phenyl)thiophen-2-yl)-1,1-dioxo-3,4,5,6-tetrahydro-2H-thiopyran-2-yl)acetate

NMR (CDCl₃, δ-0.04 (9H, s), 0.72-0.86 (2H, m), 1.71-2.23 (4H, m), 2.47 (3H, s), 2.69-2.90 (2H, m), 3.01-3.21 (2H, m), 3.14 (1H, d, J=15 Hz), 3.42 (1H, d, J=15 Hz), 3.93-4.06 (2H, m), 7.27 (2H, m), 7.53-7.63 (3H, m), 7.68 (1H, s), 7.81 (1H, d, J=8 Hz)

### EXAMPLE 126

2-(Trimethylsilyl)ethyl ((2S)-2-(5-(3-methyl-4-(oxazol-5-yl)phenyl)thiophen-2-yl)-1,1-dioxo-3,4,5,6-tetrahydro-2H-thiopyran-2-yl)acetate

NMR (CDCl₃, δ): -0.04 (9H, s), 0.78-0.86 (2H, m), 1.78-2.27 (4H, m), 2.52 (3H, s), 2.72-2.93 (2H, m), 3.06-3.23 (2H, m), 3.13 (1H, d, J=15 Hz), 3.43 (1H, d, J=15 Hz), 3.97-4.08 (2H, m), 7.22-7.32 (3H, m), 7.52 (2H, m), 7.69 (1H, d, J=8 Hz), 7.97 (1H, s)

### EXAMPLE 127

2-(Trimethylsilyl)ethyl ((2S)-2-(5-(4-(4-chlorophenyl)phenyl)thiophen-2-yl)-1,1-dioxo-3,4,5,6-tetrahydro-2H-thiopyran-2-yl)acetate

NMR (CDCl₃, δ-0.04 (9H, s), 0.78-0.87 (2H, m), 1.74-2.05 (2H, m), 2.06-2.26 (2H, m), 2.71-2.91 (2H, m), 3.01-3.21 (2H, m), 3.14 (1H, d, J=15 Hz), 3.42 (1H, d, J=15 Hz), 3.98-4.06 (2H, m), 7.27 (2H, m), 7.41 (2H, d, J=8 Hz), 7.51-7.56 (4H, m), 7.64 (2H, d, J=8 Hz)

### EXAMPLE 128

2-(Trimethylsilyl)ethyl ((2S)-2-(5-(4-(4-ethylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.72-0.90 (2H, m), 1.24 (3H, t, J=7 Hz), 1.71-2.24 (4H, m), 2.69 (2H, q, J=7 Hz), 2.71-2.92 (2H, m), 2.99-3.20 (2H, m), 3.11 (1H, d, J=15 Hz), 3.41 (1H, d, J=15 Hz), 3.92-4.06 (2H, m), 7.28-7.29 (5H, m), 7.45-7.65 (5H, m)

### EXAMPLE 129

2-(Trimethylsilyl)ethyl ((2S)-1,1-dioxo-2-(5-(4-(4-trifluoromethylphenyl)phenyl)thiophen-2-yl)-3,4,5,6-tetrahydro-2H-thiopyran-2-yl)acetate

NMR (CDCl₃, δ-0.04 (9H, s), 0.78-0.89 (2H, m), 1.78-2.29 (4H, m), 2.72-2.95 (2H, m), 3.03-3.23 (2H, m), 3.14 (1H, d, J=15 Hz), 3.45 (1H, d, J=15 Hz), 3.99-4.11 (2H, m), 7.26-7.32 (2H, m), 7.60 (2H, d, J=8 Hz), 7.63-7.76 (6H, m)

### EXAMPLE 130

2-(Trimethylsilyl)ethyl ((2S)-2-(5-(4-(4-methylthiophenyl)phenyl)thiophen-2-yl)-1,1-dioxo-3,4,5,6-tetrahydro-2H-thiopyran-2-yl)acetate

NMR (CDCl₃, δ): -0.04 (9H, s), 0.75-0.84 (2H, m), 1.78-2.25 (4H, m), 2.49 (3H, s), 2.69-2.91 (2H, m), 3.02-3.21 (2H, m), 3.14 (1H, d, J=15 Hz), 3.41 (1H, d, J=15 Hz), 3.94-4.04 (2H, m), 7.26-7.36 (4H, m), 7.45-7.74 (6H, m)

### EXAMPLE 131

2-(Trimethylsilyl)ethyl ((2S)-2-(5-(4-(4-acetylphenyl)phenyl)thiophen-2-yl)-1,1-dioxo-3,4,5,6-tetrahydro-2H-thiopyran-2-yl)acetate

NMR (CDCl₃, δ): -0.03 (9H, s), 0.74-0.86 (2H, m), 1.76-2.25 (4H, m), 2.62 (3H, s), 2.70-2.90 (2H, m), 3.02-3.22 (2H, m), 3.14 (1H, d, J=15 Hz), 3.43 (1H, d, J=15 Hz), 3.96-4.12 (2H, m), 7.27 (1H, d, J=3 Hz), 7.29 (1H, d, J=3 Hz), 7.59-7.72 (6H, m), 8.01 (2H, d, J=8 Hz)

### EXAMPLE 132

2-(Trimethylsilyl)ethyl ((2S)-2-(5-(4-(4-t-butylphenyl)phenyl)thiophen-2-yl)-1,1-dioxo-3,4,5,6-tetrahydro-2H-thiopyran-2-yl)acetate

NMR (CDCl₃, δ): −0.05 (9H, s), 0.75-0.89 (2H, m), 1.37 (9H, s), 1.75-2.30 (4H, m), 2.68-2.97 (2H, m), 3.00-3.23 (2H, m), 3.14 (1H, d, J=15 Hz), 3.45 (1H, d, J=15 Hz), 3.94-4.10 (2H, m), 7.27 (2H, m), 7.48 (2H, d, J=8 Hz), 7.50-7.68 (6H, m)

### EXAMPLE 133

2-(Trimethylsilyl)ethyl ((2S)-2-(5-(4-(4-fluorophenyl)phenyl)thiophen-2-yl)-1,1-dioxo-3,4,5,6-tetrahydro-2H-thiopyran-2-yl)acetate

NMR (CDCl₃, δ): 0.04 (9H, s), 0.78-0.89 (2H, m), 1.79-2.28 (4H, m), 2.72-2.93 (2H, m), 3.04-3.22 (2H, m), 3.14 (1H, d, J=15 Hz), 3.44 (1H, d, J=15 Hz), 3.98-4.10 (2H, m), 7.14 (2H, t, J=8 Hz), 7.24-7.31 (2H, m), 7.52-7.60 (4H, m), 7.65 (2H, d, J=8 Hz)

MS (m/z): 543 (M−H)

### EXAMPLE 134

2-(Trimethylsilyl)ethyl ((2S)-2-(5-(4-(4-methoxyphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.71-0.84 (2H, m), 1.72-2.24 (4H, m), 2.64-2.90 (2H, m), 2.99-3.19 (2H, m), 3.14 (1H, d, J=15 Hz), 3.39 (1H, d, J=15 Hz), 3.82 (3H, s), 3.91-4.04 (2H, m), 6.94 (2H, d, J=8 Hz), 7.22 (2H, m), 7.52 (4H, d, J=8 Hz), 7.60 (2H, d, J=8 Hz)

### EXAMPLE 135

2-(Trimethylsilyl)ethyl ((2S)-2-(5-(4-(4-hydroxyphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.92 (2H, m), 1.79-2.28 (4H, m), 2.74-2.96 (2H, m), 3.06-3.27 (2H, m), 3.14 (1H, d, J=15 Hz), 3.47 (1H, d, J=15 Hz), 3.98-4.10 (2H, m), 5.50 (1H, s), 6.85 (2H, d, J=8 Hz), 7.26 (2H, m), 7.38-7.49 (4H, m), 7.57 (2H, d, J=8 Hz)

### EXAMPLE 136

2-(Trimethylsilyl)ethyl ((2S)-1,1-dioxo-2-(5-(4-(4-trifluoromethoxyphenyl)phenyl)thiophen-2-yl)-3,4,5,6-tetrahydro-2H-thiopyran-2-yl)acetate

NMR (CDCl₃, δ): −0.04 (9H, s), 0.76-0.93 (2H, m), 1.76-2.28 (4H, m), 2.77-2.96 (2H, m), 3.02-3.23 (2H, m), 3.15 (1H, d, J=15 Hz), 3.43 (1H, d, J=15 Hz), 3.97-4.08 (2H, m), 7.26-7.35 (4H, m), 7.52-7.70 (6H, m)

### EXAMPLE 137

2-(Trimethylsilyl)ethyl ((2S)-2-(5-(4-(4-methylcarbamoylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.79-0.90 (2H, m), 1.77-2.30 (4H, m), 2.72-2.93 (2H, m), 3.05 (3H, d, J=7 Hz), 3.10-3.23 (2H, m), 3.15 (1H, d, J=15 Hz), 3.46 (1H, d, J=15 Hz), 3.96-4.10 (2H, m), 6.21 (1H, br), 7.27 (1H, d, J=3 Hz), 7.30 (1H, d, J=3 Hz), 7.59-7.72 (6H, m), 7.84 (2H, d, J=8 Hz)

### EXAMPLE 138

2-(Trimethylsilyl)ethyl ((2S)-2-(5-(4-(4-dimethylcarbamoylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.77-0.89 (2H, m), 1.76-2.27 (4H, m), 2.73-2.96 (2H, m), 3.00-3.23 (9H, m), 3.46 (1H, d, J=15 Hz), 3.98-4.08 (2H, m), 7.26-7.32 (2H, m), 7.44-7.54 (2H, m), 7.54-7.72 (6H, m)

MS (m/z) 598 (M+H)

### EXAMPLE 139

2-(Trimethylsilyl)ethyl ((2S)-2-(5-(4-(4-carbamoylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.73-0.95 (2H, m), 1.78-2.28 (4H, m), 2.71-2.95 (2H, m), 3.06-3.24 (2H, m), 3.15 (1H, d, J=15 Hz), 3.45 (1H, d, J=15 Hz), 4.00-4.12 (2H, m), 7.27 (1H, d, J=3 Hz), 7.29 (1H, d, J=3 Hz), 7.58-7.72 (6H, m), 7.89 (2H, d, J=8 Hz)

### EXAMPLE 140

2-(Trimethylsilyl)ethyl ((2S)-2-(5-(4-(4-methylsulfonylaminocarbonylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.93 (2H, m), 1.81-2.11 (2H, m), 2.12-2.31 (2H, m), 2.81-2.94 (2H, m), 3.06-3.28 (2H, m), 3.15 (1H, d, J=15 Hz), 3.39 (3H, br), 3.48 (1H, d, J=15 Hz), 4.01-4.12 (2H, m), 7.27 (2H, m), 7.32-7.71 (6H, m), 7.92 (2H, d, J=8 Hz)

MS (m/z) 646 (M−H)

### EXAMPLE 141

((2S)-2-(5-(4-(4-Hydroxymethylphenyl)phenyl)thiophen-2-yl)-1,1-dioxo-3,4,5,6-tetrahydro-2H-thiopyran-2-yl)acetic acid

NMR (DMSO-d₆, δ): 1.72-2.14 (4H, m), 2.40-2.58(1H, m), 2.80-2.92 (1H, m), 3.15-3.28 (2H, m), 3.50-3.65 (2H, m), 4.55 (2H, s), 7.21 (1H, d, J=3 Hz), 7.42 (2H, d, J=8 Hz), 7.53 (1H, d, J=3 Hz), 7.68 (2H, d, J=8 Hz), 7.74 (4H, s), 12.58 (1H, br)

MS (m/z) 455 (M−H)

### EXAMPLE 142

4-Methoxybenzyl ((2S)-2-(5-(4-(4-(oxazol-5-yl)phenyl)phenyl)thiophen-2-yl)-1,1-dioxo-3,4,5,6-tetrahydro-2H-thiopyran-2-yl)acetate

NMR (CDCl₃, δ): 1.76-2.03 (2H, m), 2.07-2.23 (2H, m), 2.65-2.88 (2H, m), 3.03-3.14 (2H, m), 3.16 (1H, d, J=15 Hz), 3.48 (1H, d, J=15 Hz), 3.68 (3H, s), 4.83-4.96 (2H, m), 6.73 (2H, d, J=8 Hz), 7.03 (2H, d, J=8 Hz), 7.19 (1H, d, J=3 Hz), 7.24 (1H, d, J=3 Hz), 7.41 (1H, s), 7.58-7.76 (8H, m), 7.95 (1H, s)

### EXAMPLE 143

2-(Trimethylsilyl)ethyl ((2S)-2-(5-(4-(4-t-butoxycarbonylaminophenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.88 (2H, m), 1.54 (9H, s), 1.80-2.25 (4H, m), 2.72-2.95 (2H, m), 3.05-3.25 (2H, m), 3.14 (1H, d, J=15 Hz), 3.44 (1H, d, J=15 Hz), 3.98-4.07 (2H, m), 6.54 (1H, s), 7.26 (2H, m), 7.43 (2H, d, J=8 Hz), 7.52-7.58 (4H, m), 7.63 (2H, d, J=8 Hz)

### EXAMPLE 144

4-Methoxybenzyl ((2S)-2-(5-(4-(4-(t-butoxycarbonylamino)phenyl)phenyl)thiophen-2-yl)-1,1-dioxo-3,4,5,6-tetrahydro-2H-thiopyran-2-yl)acetate

NMR (CDCl₃, δ): 1.55 (9H, s), 1.78-2.05 (2H, m), 2.10-2.23 (2H, m), 2.66-2.88 (2H, m), 3.06-3.13 (2H, m), 3.15 (1H, d, J=15 Hz), 3.48 (1H, d, J=15 Hz), 3.66 (3H, s), 4.84-4.96 (2H, m), 6.53 (1H, br), 6.72 (2H, d, J=8 Hz), 7.04 (2H, d, J=8 Hz), 7.19 (2H, m), 7.43 (2H, d, J=8 Hz), 7.53-7.61 (6H, m)

### EXAMPLE 145

2-(Trimethylsilyl)ethyl ((2S)-2-(5-(4-(4-methylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.88 (2H, m), 1.79-2.09 (2H, m), 2.10-2.23 (2H, m), 2.45 (3H, s), 2.71-2.92 (2H, m), 3.01-3.19 (2H, m), 3.13 (1H, d, J=15 Hz), 3.43 (1H, d, J=15 Hz), 3.98-4.06 (2H, m), 7.27 (2H, m), 7.48-7.68 (8H, m)

### EXAMPLE 146

2-(Trimethylsilyl)ethyl ((2S)-2-(5-(4-(3-methylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.91 (2H, m), 1.79-2.06 (2H, m), 2.09-2.29 (2H, m), 2.43 (3H, s), 2.69-2.98 (2H, m), 3.01-3.21 (2H, m), 3.15 (1H, d, J=15 Hz), 3.45 (1H, d, J=15 Hz), 3.99-4.09 (2H, m), 7.16 (1H, m), 7.26 (2H, m), 7.32 (1H, m), 7.40 (2H, m), 7.53-7.68 (4H, m)

### EXAMPLE 147

2-(Trimethylsilyl)ethyl ((2S)-2-(5-(4-(2-methylphenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.76-0.91 (2H, m), 1.78-2.26 (4H, m), 2.05 (3H, s), 2.70-2.98 (2H, m), 3.01-3.22 (2H, m), 3.14 (1H, d, J=15 Hz), 3.44 (1H, d, J=15 Hz), 3.98-4.08 (2H, m), 7.22-7.40 (8H, m), 7.62 (2H, d, J=8 Hz)

### EXAMPLE 148

2-(Trimethylsilyl)ethyl ((2S)-1,1-dioxo-2-(5-(4-(2-phenyltetrazol-5-yl)phenyl)thiophen-2-yl)-3,4,5,6-tetrahydro-2H-thiopyran-2-yl)acetate

NMR (CDCl₃, δ): −0.04 (9H, s), 0.78-0.90 (2H, m), 1.80-2.28 (4H, m), 2.74-2.96 (2H, m), 3.06-3.25 (2H, m), 3.14 (1H, d, J=15 Hz), 3.46 (1H, d, J=15 Hz), 3.99-4.10 (2H, m), 7.28 (1H, d, J=3 Hz), 7.36 (1H, d, J=3 Hz), 7.46-7.65 (3H, m), 7.75 (2H, d, J=8 Hz), 8.16-8.30 (4H, m)

### EXAMPLE 149

4-Methoxybenzyl ((2S)-2-(5-(4-((E)-2-(oxazol-5-yl)ethenyl)phenyl)thiophen-2-yl)-1,1-dioxo-3,4,5,6-tetrahydro-2H-thiopyran-2-yl)acetate

NMR (CDCl₃, δ): 1.80-2.04 (2H, m), 2.08-2.23 (2H, m), 2.65-2.90 (2H, m), 3.06-3.15 (2H, m), 3.18 (1H, d, J=15 Hz), 3.47 (1H, d, J=15 Hz), 3.69 (3H, s), 4.86-4.97 (2H, m), 6.72 (2H, d, J=8 Hz), 6.94 (1H, d, J=15 Hz), 7.03-7.10 (4H, m), 7.17-7.23 (2H, m), 7.48 (2H, d, J=8 Hz), 7.58 (2H, d, J=8 Hz), 7.86 (1H, s)

MS (m/z) 564 (M+H)

### EXAMPLE 150

4-Methoxybenzyl ((2S)-2-(5-(4-(oxazol-5-ylmethoxy)phenyl)thiophen-2-yl)-1,1-dioxo-3,4,5,6-tetrahydro-2H-thiopyran-2-yl)acetate

NMR (CDCl₃, δ): 1.74-2.03 (2H, m), 2.04-2.21 (2H, m), 2.61-2.85 (2H, m), 3.03-3.13 (2H, m), 3.14 (1H, d, J=15 Hz), 3.46 (1H, d, J=15 Hz), 3.71 (3H, s), 4.91 (2H, m), 5.11 (2H, s), 6.72 (2H, d, J=8 Hz), 6.93-7.19 (5H, m), 7.40-7.70 (4H, m), 7.93 (1H, s)

### EXAMPLE 151

4-Methoxybenzyl ((2S)-2-(5-(4-((E)-2-(methylcarbamoyl)ethenyl)phenyl)thiophen-2-yl)-1,1-dioxo-3,4,5,6-tetrahydro-2H-thiopyran-2-yl)acetate

NMR (CDCl₃, δ): 1.76-2.04 (2H, m), 2.08-2.26 (2H, m), 2.66-2.97 (2H, m), 2.96 (3H, d, J=7 Hz), 3.02-3.17 (2H, m), 3.18 (1H, d, J=15 Hz), 3.48 (1H, d, J=15 Hz), 3.67 (3H, s), 4.85-4.98 (2H, m), 5.63 (1H, br), 6.39 (1H, d, J=15 Hz), 6.70 (2H, d, J=8 Hz), 7.04 (2H, d, J=8 Hz), 7.19 (2H, m), 7.52 (2H, d, J=8 Hz), 7.57 (2H, d, J=8 Hz), 7.62 (1H, d, J=15 Hz)

### EXAMPLE 152

4-Methoxybenzyl ((2S)-2-(5-(4-(oxazol-5-ylcarbonylamino)phenyl)thiophen-2-yl)-1,1-dioxo-3,4,5,6-tetrahydro-2H-thiopyran-2-yl)acetate

NMR (CDCl₃, δ): 1.76-2.04 (2H, m), 2.10-2.28 (2H, m), 2.64-2.92 (2H, m), 3.06-3.21 (2H, m), 3.16 (1H, d, J=15 Hz), 3.48 (1H, d, J=15 Hz), 3.72 (3H, s), 4.86-4.98 (2H, m), 6.74 (2H, d, J=8 Hz), 7.06 (2H, d, J=8 Hz), 7.17 (2H, m), 7.58 (2H, d, J=8 Hz), 7.67 (2H, d, J=8 Hz), 7.87 (1H, s), 8.00 (2H, s)

MS (m/z): 579 (M−H)

### EXAMPLE 153

{(2S)-1,1-Dioxo-2-[5-(4-propylphenyl)thiophen-2-yl]-3,4,5,6-tetrahydro-2H-thiopyran-2-yl}acetic acid

NMR (DMSO-d₆, δ): 0.90 (3H, t, J=7 Hz), 1.54-1.67 (2H, m), 1.70-2.07 (4H, m), 2.38-2.61 (3H, m), 2.77-2.86 (1H, m), 3.12-3.23 (2H, m), 3.45-3.61 (2H, m), 7.15 (1H, d, J=4 Hz), 7.24 (2H, d, J=8 Hz), 7.41 (1H, d, J=4 Hz), 7.55 (2H, d, J=8 Hz)

MS (ESI): m/z 391 (M−1)

### EXAMPLE 154

((2S)-1,1-Dioxo-2-{5-[(E)-2-(4-fluorophenyl)ethenyl]-thiophen-2-yl}-3,4,5,6-tetrahydro-2H-thiopyran-2-yl)acetic acid

NMR (DMSO-d₆, δ): (1.71-2.07 (4H, m), 2.36-2.49 (1H, m), 2.75-2.85 (1H, m), 3.10-3.21 (2H, m), 3.45-3.60 (2H, m), 6.98 (1H, d, J=16 Hz), 7.10 (1H, d, J=4 Hz), 7.14 (1H, d, J=4 Hz), 7.16-24 (2H, m), 7.36 (1H, d, J=16 Hz), 7.60-7.68 (2H, m)

MS (ESI): m/z 393 (M−1)

### EXAMPLE 155

2-(Trimethylsilyl)ethyl ((2S)-1,1-dioxo-2-{5-[4-(oxazol-4-yl)phenyl]thiophen-2-yl}-3,4,5,6-tetrahydro-2H-thiopyran-2-yl)acetate

NMR (CDCl₃, δ): −0.01 (9H, s), 0.75-0.85 (2H, m), 1.80-2.05 (4H, m), 1.10-2.25 (2H, m), 2.70-2.95 (2H, m), 3.05-3.20 (2H, m), 3.12 (1H, d, J=15 Hz), 3.43 (1H, d, J=15 Hz), 3.98-4.06 (2H, m), 7.26 (1H, d, J=4 Hz), 7.29 (1H, d, J=4 Hz), 7.64 (2H, d, J=9 Hz), 7.75 (2H, d, J=9 Hz), 7.95 (1H, s), 7.98 (1H, s)

### EXAMPLE 156

2-(Trimethylsilyl)ethyl ((2S)-1,1-dioxido-2-{5-[4-(thiazol-4-yl)phenyl]thiophen-2-yl}-3,4,5,6-tetrahydro-2H-thiopyran-2-yl)acetate

NMR (CDCl₃, δ): −0.01 (9H, s), 0.80-0.90 (2H, m), 1.80-2.20 (4H, m), 2.75-2.95 (2H, m), 3.05-3.25 (2H, m), 3.16 (1H, d, J=1 Hz), 3.46 (1H, d, J=15 Hz), 4.04-4.10 (2H, m), 7.31 (1H, d, J=4 Hz), 7.35 (1H, d, J=4 Hz), 7.61 (1H, d, J=2 Hz), 7.71 (2H, d, J=9 Hz), 7.98 (2H, d, J=9 Hz), 8.93 (1H, d, J=2 Hz)

### EXAMPLE 157

4-Methoxybenzyl {(2S)-1,1-dioxo-2-[5-(4-hydroxyphenyl)thiophen-2-yl]-3,4,5,6-tetrahydro-2H-thiopyran-2-yl}acetate

NMR (DMSO-d₆, δ): 1.70-2.00 (4H, m), 2.40-2.50 (1H, m), 2.60-2.70 (1H, m), 3.10-3.20 (1H, m), 3.24 (1H, d, J=15 Hz), 3.45-3.60 (1H, m), 3.65 (1H, d, J=15 Hz), 3.66 (3H, s), 4.92 (2H, s), 6.77 (2H, d, J=9 Hz), 6.81 (2H, d, J=9 Hz), 7.07 (2H, d, J=9 Hz), 7.09 (1H, d, J=4 Hz), 7.09 (1H, d, J=4 Hz), 7.23 (2H, d, J=9 Hz), 7.45 (2H, d, J=9 Hz)

### EXAMPLE 158

2-(Trimethylsilyl)ethyl {(2S)-1,1-dioxo-2-[5-(6-fluorobenzothiophen-2-yl)thiophen-2-yl]-3,4,5,6-tetrahydro-2H-thiopyran-2-yl}acetate

NMR (CDCl₃, δ): −0.05 (9H, s), 0.80-0.90 (2H, m), 1.80-2.30 (4H, m), 2.70-2.95 (2H, m), 3.00-3.25 (2H, m), 3.17 (1H, d, J-15 Hz), 3.45 (1H, d, J=15 Hz), 4.02-4.15 (2H, m), 7.13 (1H, dt, J=2, 9 Hz), 7.20-7.30 (3H, m), 7.40 (1H, s), 7.50 (1H, dd, J=2, 9 Hz), 7.69 (1H, dd. J=5, 9 Hz)

### EXAMPLE 159

((2S)-1,1-Dioxo-2-{5-[4-(thiophen-2-yl)phenyl]-thiophen-2-yl}-3,4,5,6-tetrahydro-2H-thiopyran-2-yl)acetic acid

NMR (DMSO-d₆, δ): 1.80-2.19 (4H, m), 2.48-2.60(1H, m), 2.86-2.96 (1H, m), 3.20-3.35 (2H, m), 3.60-3.72 (2H, m), 7.22-7.26 (1H, m), 7.29 (1H, d, J=4 Hz), 7.60 (1H, d, J=4 Hz), 7.64-7.69 (2H, m), 7.74-7.83 (4H, m)

MS (ESI−): 431 (M−H)

The following compounds were obtained in a similar manner to those of Preparation 2-2) and Example 101.

### EXAMPLE 160

{(2S)-1,1-Dioxo-2-[5-(3-methyl-2-oxo-2,3-dihydro-1,3-benzothiazol-6-yl)thiophen-2-yl]-3,4,5,6-tetrahydro-2H-thiopyran-2-yl}acetic acid

NMR (DMSO-d₆, δ): 1.72-2.11 (4H, m), 2.37-2.47 (1H, m), 2.77-2.90 (1H, m), 3.12-3.24 (2H, m), 3.42 (3H, s), 3.48-3.63 (2H, m), 7.17 (1H, d, J=4 Hz), 7.35 (1H, d, J=8 Hz), 7.45 (1H, d, J=4 Hz), 7.66 (1H, d, J=8 Hz), 8.04 (1H, s)

MS (ESI): m/z 436 (M−1)

### EXAMPLE 161

((2S)-1,1-Dioxo-2-{5-[4-(5-methyl-1,2,4-oxadiazol-3-yl)phenyl]thiophen-2-yl}-3,4,5,6-tetrahydro-2H-thiopyran-2-yl)acetic acid

NMR (DMSO-d₆, δ): 1.71-2.09 (4H, m), 2.41-2.48 (1H, m), 2.68 (3H, d, J=5 Hz), 2.81-2.88 (1H, m), 3.15-3.23 (2H, m), 3.48-3.62 (2H, m), 7.23 (1H, d, J=4 Hz), 7.62 (1H, d, J=4 Hz), 7.84 (1H, d, J=8 Hz), 7.97 (1H, d, J=8 Hz), 8.02 (1H, d, J=8 Hz), 8.12 (1H, d, J=8 Hz)

MS (ESI): m/z 431 (M−1)

### EXAMPLE 162

To a crude mixture of 4-(5-[(2S)-2-(tert-butoxycarbonylmethyl)-1,1-dioxo-3,4,5,6-tetrahydro-2H-thiopyran-2-yl]thiophen-2-yl)phenylboronic acid obtained in Preparation 69 was added a solution of 4-bromopyridine hydrochloride (715 mg) in dioxane (10 ml), dichlorobis(triphenylphosphine)palladium(II) (15.4 mg) and 2M sodium carbonate (3 ml) at ambient temperature. After being stirred at 80° C. for 3 hours, the mixture was concentrated in vacuo. The residue was dissolved in AcOEt (10 ml) and the solution was washed with water, 0.5M HCl, 1M NaHCO₃ (Sodium bicarbonate) and brine, dried over MgSO₄, and concentrated in vacuo. The residue was purified by purified by SiO₂ column chromatography (hexane/EtOAc, 1:2) to give tert-butyl ((2S)-1,1-dioxo-2-{[5-[4-(pyridin-4-yl)phenyl]thiophen-2-yl}-3,4,5,6-tetrahydro-2H-thiopyran-2-yl)acetate (136 mg) as a powder.

NMR (CDCl₃, δ): 1.27 (9H, s), 1.80-2.05 (2H, m), 2.08-2.25 (2H, m), 2.70-2.88 (2H, m), 3.02-3.18 (3H, m), 3.38 (1H, d, J=16 Hz), 7.26 (1H, d, J=4 Hz), 7.34 (1H, d, J=4 Hz), 7.42-7.77 (6H, m), 8.67 (2H, d, J=8 Hz)

MS (ESI+): 484(M+H)

The following compounds were obtained in a similar manner to that of Example 162.

### EXAMPLE 163

tert-Butyl ((2S)-1,1-dioxo-2-{[5-[4-(thiophen-3-yl)phenyl]thiophen-2-yl}-3,4,5,6-tetrahydro-2H-thiopyran-2-yl)acetate

NMR (CDCl₃, δ): 1.26 (9H, s), 1.81-2.05 (2H, m), 2.09-2.22 (2H, m), 2.68-2.88 (2H, m), 3.02-3.20 (3H, m), 3.37 (1H, d, J=16 Hz), 7.22-7.32 (3H, m), 7.38-7.44 (1H, m), 7.50-7.72 (5H, m)

MS (ESI+): 433 (M+H)

### EXAMPLE 164

tert-Butyl ((2S)-1,1-dioxo-2-{[5-[4-(thiazol-2-yl)phenyl]thiophen-2-yl}-3,4,5,6-tetrahydro-2H-thiopyran-2-yl)acetate

NMR (CDCl₃, δ): 1.26 (9H, s), 1.80-2.25 (4H, m), 2.67-2.87 (2H, m), 3.00-3.18 (3H, m), 3.38 (1H, d, J=16 Hz), 7.27 (1H, d, J=4 Hz), 7.35 (1H, d, J=4 Hz), 7.62-7.76 (3H, m), 7.84-8.02 (3H, m)

MS (ESI+): 490 (M+H)

### EXAMPLE 165

tert-Butyl ((2S)-1,1-dioxo-2-{[5-[4-(pyridin-3-yl)phenyl]thiophen-2-yl}-3,4,5,6-tetrahydro-2H-thiopyran-2-yl)acetate

NMR (CDCl₃, δ): 1.27 (9H, s), 1.72-2.25 (4H, m), 2.68-2.88 (2H, m), 3.02-3.19 (3H, m), 3.38 (1H, d, J=16 Hz), 7.27 (1H, d, J=4 Hz), 7.34-7.41 (1H, m), 7.59 (2H, d, J=8 Hz), 7.65-7.75 (3H, m), 7.87-7.96 (1H, m), 8.60 (1H, d, J=4 Hz), 8.85-8.94 (1H, m)

MS (ESI−): 482 (M−H)

### EXAMPLE 166

tert-Butyl (2S)-1,1-dioxo-2-{[5-[4-(pyridin-2-yl)phenyl]thiophen-2-yl}-3,4,5,6-tetrahydro-2H-thiopyran-2-yl)acetate

NMR (CDCl₃, δ): 1.26 (9H, s), 1.80-2.06 (2H, m), 2.10-2.24 (2H, m), 2.66-2.88 (2H, m), 3.02-3.18 (3H, m), 3.38 (1H, d, J=16 Hz), 7.26 (1H, d, J=4 Hz), 7.34 (1H, d, J=4 Hz), 7.66-7.80 (5H, m), 8.02 (2H, d, J=8 Hz), 8.70 (1H, d, J=4 Hz)

MS (ESI+): 484 (M+H)

### EXAMPLE 167

2-(Trimethylsilyl)ethyl ((2S)-2-(5-(4-(3-cyanophenyl)phenyl)thiophen-2-yl)-1,1-dioxo-3,4,5,6-tetrahydro-2H-thiopyran-2-yl)acetate

NMR (CDCl₃, δ): −0.04 (9H, s), 0.78-0.91 (2H, m), 1.81-2.30 (4H, m), 2.72-2.96 (2H, m), 3.06-3.24 (2H, m), 3.14 (1H, d, J=15 Hz), 3.44 (1H, d, J=15 Hz), 4.00-4.12 (2H, m), 7.28 (1H, d, J=3 Hz), 7.32 (1H, d, J=3 Hz), 7.51-7.75 (6H, m), 7.81-7.93 (2H, m)

### EXAMPLE 168

[(2S)-2-(5-{(E)-2-[4-(tert-Butoxycarbonyloxy)phenyl]-ethenyl}thiophen-2-yl)-1,1-dioxo-3,4,5,6-tetrahydro-2H-thiopyran-2-yl]acetic acid (350 mg) was dissolved in ethyl acetate (6 ml) and 4N hydrogenchloride in ethyl acetate (2 ml) was added. The mixture was stirred for 2 hours at 20° C. and partitioned between ethyl acetate and water. The organic layer was separated, washed with water and brine, dried over magnesium sulfate, and evaporated in vacuo. The residue was purified by preparative thin-layer chromatograpy (20% methanol in chloroform) and triturated with ether to give ((2S)-1,1-dioxo-2-{5-[(E)-2-(4-hydroxyphenyl)ethenyl]thiophen-2-yl}-3,4,5,6-tetrahydro-2H-thiopyran-2-yl)acetic acid (70 mg) as a yellow powder.

NMR (CDCl₃, δ): 1.70-1.90 (4H, m), 2.35-2.50 (1H, m), 2.75-2.85 (1H, m), 3.10-3.20 (2H, m), 3.45-3.60 (2H, m), 6.75 (2H, d, J=9 Hz), 6.85 (1H, d, J=18 Hz), 7.00 (1H, d, J=4 Hz), 7.01 (1H, d, J=4 Hz), 7.15 (1H, d, J=18 Hz), 7.39 (2H, d, J=9 Hz)

### EXAMPLE 169

To a solution of 2-(trimethylsilyl)ethyl ((2S)-2-(5-(4-(4-t-butoxycarbonylaminophenyl)phenyl)thiophen-2-yl)-1,1-dioxo-3,4,5,6-tetrahydro-2H-thiopyran-2-yl)acetate (150 mg) in ethyl acetate (3 ml) was added 4N hydrochloric acid ethyl acetate solution (3 ml) and the mixture was stirred at ambient temperature for 2 hours. The solution was washed with saturated sodium bicarbonate solution and brine, dried over magnesium sulfate, and evaporated in vacuo to give 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate as a yellow powder (120 mg).

NMR (CDCl₃, δ): −0.04 (9H, s), 0.77-0.88 (2H, m), 1.78-2.30 (4H, m), 2.70-2.96 (2H, m), 3.00-3.25 (2H, m), 3.12 (1H, d, J=15 Hz), 3.43 (1H, d, J=15 Hz), 3.97-4.07 (2H, m), 6.76 (2H, d, J=8 Hz), 7.26 (2H, m), 7.43 (2H, d, J=8 Hz), 7.53 (2H, d, J=8 Hz), 7.61 (2H, d, J=8 Hz)

### EXAMPLE 170

To a solution of 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate (114 mg) in dichloromethane (3 ml) was added acetic anhydride (25.8 mg) and the mixture was stirred at ambient temperature for 1 hour. The solution was washed with water, 1N hydrochloric acid, water, saturated sodium bicarbonate solution and brine, dried over magnesium sulfate, and evaporated in vacuo. The residue was purified by silica gel column chromatography eluting with a mixture of hexane-ethyl acetate (3:1-1:3) to give 2-(trimethylsilyl)ethyl ((2S)-2-(5-(4-(4-acetamidophenyl)phenyl)thiophen-2-yl)-1,1-dioxo-3,4,5,6-tetrahydro-2H-thiopyran-2-yl)acetate as a yellow amorphous powder (102 mg).

NMR (CDCl₃, δ): −0.05 (9H, s), 0.78-0.93 (2H, m), 1.77-2.27 (4H, m), 2.21 (3H, s), 2.74-2.94 (2H, m), 3.05-3.25 (2H, m), 3.14 (1H, d, J=15 Hz), 3.44 (1H, d, J=15 Hz), 3.98-4.07 (2H, m), 7.26 (2H, s), 7.37 (1H, s), 7.50-7.65 (8H, m)

### EXAMPLE 171

To a solution of 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate (120 mg) in dichloromethane (3 ml) was added ethyl isocyanate (31.5 mg) and the mixture was stirred at ambient temperature for 2 hours. After evaporation of solvent, the residue was purified by silica gel column chromatography eluting with a mixture of hexane-ethyl acetate (1:1-1:5) to give 2-(trimethylsilyl)ethyl ((2S)-2-(5-(4-(4-ethylaminocarbonylaminophenyl)phenyl)thiophen-2-yl)-1,1-dioxo-3,4,5,6-tetrahydro-2H-thiopyran-2-yl)acetate as a colorless amorphous powder (112 mg).

NMR (CDCl₃, δ): −0.04 (9H, s), 0.76-0.89 (2H, m), 1.18 (3H, t, J=7 Hz), 1.79-2.08 (2H, m), 2.12-2.32 (2H, m), 2.77-2.90 (2H, m), 3.06-3.25 (2H, m), 3.14 (1H, d, J=15 Hz), 3.26-3.38 (2H, m), 3.47 (1H, d, J=15 Hz), 3.98-4.09 (2H, m), 4.92 (1H, br), 6.52 (1H, br), 7.26 (2H, m), 7.32 (2H, d, J=8 Hz), 7.42-7.50 (4H, m), 7.55 (2H, d, J=8 Hz)

### EXAMPLE 172

To a solution of 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate (128 mg) and triethylamine (35.9 mg) in dichloromethane (3 ml) was added methanesulfonyl choride (28.4 mg) under ice-water cooling and the mixture was stirred at ambient temperature for 2 hours. The solution was washed with water, 1N hydrochloric acid, water, saturated sodium bicarbonate solution and brine, dried over magnesium sulfate, and evaporated in vacuo. The residue was purified by preparative silica gel column chromatography eluting with a mixture of chloroform and methanol (20:1) to give 2-(trimethylsilyl)ethyl ((2S)-2-(5-(4-(4-methylsulfonylaminophenyl)phenyl)thiophen-2-yl)-1,1-dioxo-3,4,5,6-tetrahydro-2H-thiopyran-2-yl)acetate as a pale yellow amorphous powder (112 mg).

NMR (CDCl₃, δ): −0.04 (9H, s), 0.78-0.91 (2H, m), 1.78-2.28 (4H, m), 2.77-2.89 (2H, m), 3.04 (3H, s), 3.11-3.28 (2H, m), 3.14 (1H, d, J=15 Hz); 3.46 (1H, d, J=15 Hz), 4.00-4.11 (2H, m), 6.66 (1H, br), 7.26 (4H, m), 7.46-7.58 (4H, m), 7.62 (2H, d, J=8 Hz)

### EXAMPLE 173

To a solution of 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate (130 mg) in chloroform (3 ml) and pyridine (3 ml) was added methyl chloroformate (23.8 mg) under ice-water cooling and the mixture was stirred at ambient temperature for 2 hours. The solution was washed with water, 1N hydrochloric acid, water, saturated sodium bicarbonate solution and brine, dried over magnesium sulfate, and evaporated in vacuo. The residue was purified by preparative silica gel column chromatography eluting with a mixture of hexane and ethyl acetate (3:1-1:2) to give 2-(trimethylsilyl)ethyl ((2S)-2-(5-(4-(4-methoxycarbonylaminophenyl)phenyl)thiophen-2-yl)-1,1-dioxo-3,4,5,6-tetrahydro-2H-thiopyran-2-yl)acetate as yellow oil (143 mg).

NMR (CDCl₃, δ): −0.04 (9H, s), 0.77-0.88 (2H, m), 1.78-2.28 (4H, m), 2.70-2.95 (2H, m), 3.03-3.25 (2H, m), 3.14 (1H, d, J=15 Hz), 3.44 (1H, d, J=15 Hz), 3.80 (3H, s), 4.00-4.08 (2H, m), 6.68 (1H, br), 7.27 (2H, m), 7.45 (2H, d, J=8 Hz), 7.52-7.67 (6H, m)

### EXAMPLE 174

A mixture of 4-methoxybenzyl ((2S)-1,1-dioxo-2-[5-(4-hydroxyphenyl)thiophen-2-yl]-3,4,5,6-tetrahydro-2H-thiopyran-2-yl)acetate (200 mg), 1-iodobutane (91 mg) and potassium carbonate (114 mg) in N,N-dimethylformamide (4 ml) was stirred at 50° C. for 1 hour, then at 60° C. for 1 hour. The mixture was partitioned between ethyl acetate and 1N hydrochloric acid. The organic layer was separated, washed with water and brine, dried over magnesium sulfate, and evaporated in vacuo. The residue was purified by preparative thin-layer chromatograpy (hexane/EtOAc=1:1) and triturated with isopropyl ether to give 4-methoxybenzyl {(2S)-2-[5-(4-butoxyphenyl)thiophen-2-yl]-1,1-dioxo-3,4,5,6-tetrahydro-2H-thiopyran-2-yl}acetate (171 mg) as colorless crystals.

NMR (DMSO-d₆, δ): 0.99 (3H, t, J=7 Hz), 1.47-1.56 (2H, m), 1.75-2.00 (4H, m), 2.05-2.22 (2H, m), 2.60-2.85 (2H, m), 3.00-3.15 (2H, m), 3.14 (1H, d, J=15 Hz), 3.46 (1H, d, J=15 Hz), 3.70 (3H, s), 3.99 (2H, t, J=7 Hz), 4.87 (1H, d, J=10 Hz), 4.92 (1H, d, J=10 Hz), 6.72 (2H, d, J=9 Hz), 6.89 (2H, d, J=9 Hz), 7.04 (2H, d, J=9 Hz), 7.06 (1H, d, J=4 Hz), 7.14 (1H, d, J=4 Hz), 7.48 (2H, d, J=9 Hz)

### EXAMPLE 175

4-Methoxybenzyl {(2S)-1,1-dioxo-2-[5-(4-pentyloxyphenyl)thiophen-2-yl]-3,4,5,6-tetrahydro-2H-thiopyran-2-yl}acetate was obtained in a similar manner to that of Example 174.

NMR (CDCl₃, δ): 0.94 (3H, t, J=7 Hz), 1.30-1.50 (4H, m), 1.75-2.00 (4H, m), 2.05-2.20 (2H, m), 2.65-2.85 (2H, m), 3.00-3.15 (2H, m), 3.14 (1H, d, J=15 Hz), 3.46 (1H, d, J=15 Hz), 3.70 (3H, s), 3.98 (2H, t, J=7 Hz), 4.87 (1H, d, J=10 Hz), 4.92 (1H, d, J=10 Hz), 6.72 (2H, d, J=9 Hz), 6.89 (2H, d, J=9 Hz), 7.04 (2H, d, J=9 Hz), 7.06 (1H, d, J=4 Hz), 7.14 (1H, d, J=4 Hz), 7.48 (2H, d, J=9 Hz)

### EXAMPLE 176

4-Methoxybenzyl ((2S)-1,1-dioxo-2-{5-[4-(1-methylethoxy)phenyl]thiophen-2-yl}-3,4,5,6-tetrahydro-2H-thiopyran-2-yl)acetate was obtained in a similar manner to that of Example 174.

NMR (CDCl₃, δ): 1.35 (6H, d, J=7 Hz), 1.80-2.20 (4H, m), 2.50-3.00 (2H, m), 3.00-3.20 (2H, m), 3.13 (1H, d, J=15 Hz), 3.46 (1H, d, J=15 Hz), 3.70 (3H, s), 4.58 (1H, septet, J=7 Hz), 4.89 (1H, d, J=15 Hz), 4.91 (1H, d, J=15 Hz), 6.72 (2H, d, J=9 Hz), 6.88 (2H, d, J=9 Hz), 7.03 (2H, d, J=9 Hz), 7.06 (1H, d, J=4 Hz), 7.14 (1H, d, J=4 Hz), 7.47 (2H, d,-J=9 Hz)

### EXAMPLE 177

To a mixture of [(2S)-2-(5-Bromothiophen-2-yl)-1,1-dioxo-3,4,5,6-tetrahydro-2H-thiopyran-2-yl]acetic acid (200 mg), PdCl₂(Ph₃, P)₂ (19.9 mg), cur (5.39 mg) and iPr₂NH (diisopropylamide) (14 ml) was added ethynylbenzene (289 mg), and the mixture was refluxed for 3 hours. After cooling, the reaction mixture was acidified with 1N—HCl and extracted with AcOEt. The organic layer was dried over MgSO₄ and evaporated in vacuo. The residue was purified by chromatography on silica gel to give {(2S)-1,1-dioxo-2-[5-(phenylethynyl)thiophen-2-yl]-3,4,5,6-tetrahydro-2H-thiopyran-2-yl}acetic acid (130 mg).

NMR (DMSO-d₆, δ): 1.69-2.08 (4H, m), 2.35-2.53 (1H, m), 2.72-2.83 (1H, m), 3.12-3.23 (2H, m), 3.48-3.61 (2H, m), 7.18 (1H, d, J=4 Hz), 7.35 (1H, d, J=4 Hz), 7.41-7.47 (3H, m), 7.53-7.58 (2H, m)

MS (ESI): m/z 373 (M−1)

The following compounds were obtained in a similar manner to that of Example 177.

### EXAMPLE 178

((2S)-1,1-Dioxo-2-(5-[1-pentynyl]thiophen-2-yl}-3,4,5,6-tetrahydro-2H-thiopyran-2-yl)acetic acid

mp: 171-172° C.

NMR (CDCl₃, δ): 1.02 (3H, s), 1.60 (2H, sextet, J=7 Hz), 1.75-1.99 (2H, m), 2.07-2.18 (2H, m), 2.39 (2H, t, J=7 Hz), 2.61-2.84 (2H, m), 2.99-3.10 (2H, m), 3.14 (1H, d, J=15 Hz), 3.42 (1H, d, J=15 Hz), 7.04 (1H, d, J=2 Hz), 7.12 (1H, d, J=2 Hz)

MS (m/z): 339 (M⁺−H), 127 (bp)

### EXAMPLE 179

((2S)-2-{5-[(4-Chlorophenyl)ethynyl]thiophen-2-yl)-1,1-dioxo-3,4,5,6-tetrahydro-2H-thiopyran-2-yl)acetic acid

mp: 203-204° C.

NMR (CDCl₃, δ): 1.73-2.01 (2H, m), 2.07-2.20 (2H, m), 2.63-2.86 (2H, m), 3.02-3.13 (3H, m), 3.18 (1H, d, J=15 Hz), 3.44 (1H, d, J=15 Hz), 7.17-2.22 (2H, m), 7.19 (2H, s), 7.31 (2H, t, J=8 Hz), 7.40 (2H, t, J=8 Hz)

MS (m/z): 407 (M⁺−H), 127 (bp)

### EXAMPLE 180

((2S)-1,1-Dioxo-2-{5-[(4-methylphenyl)ethynyl]-thiophen-2-yl}-3,4,5,6-tetrahydro-2H-thiopyran-2-yl)acetic acid

mp: 172-173° C.

NMR (CDCl₃, δ): 1.80-2.02 (2H, m), 2.07-2.19 (2H, m), 2.37 (3H, s), 2.65-2.87 (2H, m), 3.05-3.15 (3H, m), 3.17 (1H, d, J=15 Hz), 3.44 (1H, d, J=15 Hz), 7.14 (2H, d, J=8 Hz), 7.19 (2H, s), 7.38 (2H, t, J=8 Hz)

MS (m/z): 387 (M⁺−H), 145 (bp)

### EXAMPLE 181

((2S)-1,1-Dioxo-2-{5-[(4-cyanophenyl)ethynyl]thiophen-2-yl)-3,4,5,6-tetrahydro-2H-thiopyran-2-yl}acetic acid

NMR (DMSO-d₆, δ): 1.70-2.07 (4H, m), 2.36-2.48 (1H, m), 2.74-2.83 (1H, m), 3.12-3.24 (2H, m), 3.48-3.62 (2H, m), 7.21 (1H, d, J=4 Hz), 7.45 (1H, d, J=4 Hz), 7.74 (2H, d, J=8 Hz), 7.91 (2H, d, J=8 Hz)

MS (ESI): m/z 398 (M−1)

### EXAMPLE 182

[(2S)-1,1-Dioxo-2-(5-{[4-(oxazol-5-yl)phenyl]ethynyl)thiophen-2-yl)-3,4,5,6-tetrahydro-2H-thiopyran-2-yl]acetic acid

NMR (DMSO-d₆, δ): 1.71-2.07 (4H, m), 2.36-2.54 (1H, m), 2.72-2.84 (1H, m), 3.13-3.24 (2H, m), 3.54-3.64 (2H, m), 7.19 (1H, d, J=4 Hz), 7.40 (1H, d, J=4 Hz), 7.66 (2H, d, J=8 Hz), 7.80 (2H, d, J=8 Hz), 7.82 (1H, s), 8.51 (1H, s)

MS (ESI): m/z 442 (M+1)

### EXAMPLE 183

((2S)-1,1-Dioxo-2-{5-[(4-ethoxyphenyl)ethynyl]-thiophen-2-yl)-3,4,5,6-tetrahydro-2H-thiopyran-2-yl}acetic acid

NMR (DMSO-d₆, δ): 1.33 (3H, t, J=7 Hz), 1.70-2.06 (4H, m), 2.35-2.48 (1H, m), 2.72-2.83 (1H, m), 3.10-3.23 (2H, m), 3.46-3.60 (2H, m), 4.07 (2H, q, J=7 Hz), 6.97 (2H, d, J=8 Hz), 7.15 (1H, d, J=4 Hz), 7.30 (1H, d, J=4 Hz), 7.47 (2H, d, J=8 Hz)

MS (ESI): m/z 417 (M−1)

### EXAMPLE 184

To a mixture of [(2S)-2-(5-Bromothiophen-2-yl)-1,1-dioxo-3,4,5,6-tetrahydro-2H-thiopyran-2-yl]acetic acid (200 mg), Pd(OAc)₂ (palladium(II) acetate) (6.36 mg), (o-tolyl)₃P (tri-O-tolylphosphine) (17.2 mg) and Et₃N (triethylamine) (2.4 ml) was added 4-Chlorostyrene (392 mg), and the mixture was refluxed for 2 hours. After cooling, the reaction mixture was acidified with 1N—HCl and extracted with AcOEt. The organic layer was dried over MgSO4 and evaporated in vacuo. The residue was purified by chromatography on silica gel to give ((2S)-1,1-Dioxo-2-{5-[(E)-2-(4-chlorophenyl)ethenyl]thiophen-2-yl}-3,4,5,6-tetrahydro-2H-thiopyran-2-yl)acetic acid (20 mg).

NMR (DMSO-d₆, δ): 1.69-2.06 (4H, m), 2.37-2.47 (1H, m), 2.70-2.87 (1H, m), 3.09-3.21 (2H, m), 3.46-3.58 (2H, m), 6.97 (1H, d, J=16 Hz), 7.11 (1H, d, J=4 Hz), 7.16 (1H, d, J=4 Hz), 7.42 (2H, d, J=8 Hz), 7.44 (1H, d, J=16 Hz), 7.61 (2H, d, J=8 Hz)

MS (ESI): m/z 409 (M−1)

The following compounds were obtained in a similar manner to that of Example 184.

### EXAMPLE 185

((2S)-1,1-Dioxo-2-{5-[(E)-2-(4-methoxyphenyl)ethenyl]-thiophen-2-yl)-3,4,5,6-tetrahydro-2H-thiopyran-2-yl}acetic acid

NMR (DMSO-d₆, δ): 1.68-2.06 (4H, m), 2.35-2.49 (1H, m), 2.74-2.85 (1H, m), 3.08-3.21 (2H, m), 3.42-3.60 (2H, m), 3.77 (3H, s), 6.87-7.02 (3H, m), 7.08 (2H, s), 7.25 (1H, d, J=16 Hz), 7.52 (2H, d, J=8 Hz)

MS (ESI): m/z 405 (M−1)

### EXAMPLE 186

[(2S)-2-(5-{(E)-2-[4-(tert-Butoxycarbonyloxy)phenyl]-ethenyl}thiophen-2-yl)-1,1-dioxo-3,4,5,6-tetrahydro-2H-thiopyran-2-yl]acetic acid

NMR (CDCl₃, δ): 1.57 (9H, s), 1.65-2.00 (2H, m), 2.05-2.20 (2H, m), 2.65-2.90 (2H, m), 3.00-3.25 (2H, m), 3.15 (1H, d, J=15 Hz), 3.45 (1H, d, J=15 Hz), 6.90 (1H, d, J=18 Hz), 6.98 (1H, d, J=4 Hz), 7.10 (1H, d, J=18 Hz), 7.14 (1H, d, J=4 Hz), 7.15 (2H, d, J=9 Hz), 7.43 (2H, d, J=9 Hz)

### EXAMPLE 187

{(2S)-1,1-Dioxo-2-[5-((E)-2-phenylethenyl)thiophen-2-yl]-3,4,5,6-tetrahydro-2H-thiopyran-2-yl}acetic acid was obtained in a similar manner to those of Example 184 and Example 94.

NMR (DMSO-d₆, δ): 1.70-2.07 (4H, m), 2.35-2.55 (1H, m), 2.77-2.87 (1H, m), 3.10-3.20 (2H, m), 3.50-3.58 (2H, m), 6.97 (1H, d, J=16 Hz), 7.11 (1H, d, J=4 Hz), 7.15 (1H, d, J=4 Hz), 7.23-7.45 (4H, m), 7.58 (2H, d, J=8 Hz)

MS (ESI): m/z 375 (M−1)

The following compounds were obtained in a similar manner to that of Example 187.

### EXAMPLE 188

((2S)-1,1-Dioxo-2-{5-[(E)-2-(4-methylphenyl)ethenyl]-thiophen-2-yl}-3,4,5,6-tetrahydro-2H-thiopyran-2-yl)acetic acid

NMR (DMSO-d₆, δ): 1.71-2.06 (4H, m), 2.30 (3H, s), 2.36-2.49 (1H, m), 2.76-2.86 (1H, m), 3.10-3.20 (2H, m), 3.49-3.57 (2H, m), 6.92 (1H, d, J=16 Hz), 7.09 (1H, d, J=4 Hz), 7.12 (1H, d, J=4 Hz), 7.18 (2H, d, J=8 Hz), 7.34 (1H, d, J=16 Hz), 7.47 (2H, d, J=8 Hz)

MS (ESI): m/z 389 (M−1)

### EXAMPLE 189

((2S)-1,1-Dioxo-2-{5-[(E)-2-(2-naphthyl)ethenyl]-thiophen-2-yl}-3,4,5,6-tetrahydro-2H-thiopyran-2-yl)acetic acid

NMR (DMSO-d₆, δ): 1.73-2.07 (4H, m), 2.39-2.53 (1H, m), 2.77-2.86 (1H, m), 3.13-3.22 (2H, m), 3.47-3.62 (2H, m), 7.10-7.22 (3H, m), 7.47-7.60 (3H, m), 7.83-8.02 (5H, m)

MS (ESI): m/z 425 (M−1)

### EXAMPLE 190

A mixture of benzoxazole (100 mg), 2-(trimethyl)-silylethyl [(2S)-2-(5-bromothiophen-2-yl)-1,1-dioxo-3,4,5,6-tetrahydro-2H-thiopyran-2-yl)acetate (761 mg), palladium acetate (10 mg), copper iodide(320 mg), triphenylphosphine (44 mg), sodium carbonate(356 mg) in N,N-dimethylforamide (4.2 ml) was stirred at 100° C. for 8 hours. Additionally benzoxazole (100 mg), palladium acetate (10 mg), copper iodide (320 mg), triphenylphosphine (22 mg), and sodium carbonate (178 mg) were added and the reaction mixture was stirred at 100° C. for 8 hours. After cooling, the mixture was filtered through celite and concentrated in vacuo. The residue was diluted with water and extracted with ethyl acetate. The organic layer was washed with water, brine, dried over anhydrous magnesium sulfate and concentrated in vacuo. The residue was purified by flash column chromatography on silica gel eluting with a mixture of ethyl acetate and n-hexane (1:2) to give 2-(trimethyl)silylethyl ((2S)-2-[5-(benzoxazol-2-yl)thiophen-2-yl]-1,1-dioxo-3,4,5,6-tetrahydro-2H-thiopyran-2-yl}acetate (92 mg) as a yellow crystal.

NMR (CDCl₃, δ): 0.02 (9H, s), 0.84 (2H, t, J=7 Hz), 1.75-2.00 (2H, m), 2.09-2.20 (2H, m), 2.69-2.77 (2H, m), 3.02-3.14 (3H, m), 3.33 (1H, d, J=15 Hz), 3.99-4.08 (2H, m), 7.00-7.04 (2H, m), 7.27(4H, s)

### EXAMPLE 191

To a solution of 2-(trimethylsilyl)ethyl (2S)-2-[5-[(4-methoxyphenyl)ethynyl]thiophen-2-yl]-1,1-dioxo-3,4,5,6-tetrahydro-2H-thiopyran-2-acetate (112 mg) in dioxane (1 ml) was added 1N sodium hydroxide (0.266 ml) at ambient temperature. After 1 hour, the reaction mixture was adjusted to pH 3 with 1N hydrochloric acid and water was added therein. The mixture was extracted with chloroform twice. The combined organic layer was dried over magnesium sulfate and was evaporated in vacuo. The residue was purified by preparative-TLC (chloroform-methanol=10-0) to give (2S)-2-[5-[(4-methoxyphenyl)ethynyl]thiophen-2-yl]-1,1-dioxo-3,4,5,6-tetrahydro-2H-thiopyran-2-acetic acid as a pale brown amorphous (15 mg).

NMR (CDCl₃, δ): 1.77-2.20 (4H, m), 2.62-2.81 (2H, m), 2.97-3.18 (3H, m), 3.47 (1H, d, J=15 Hz), 3.81 (3H, s), 6.85 (2H, d, J=8 Hz), 7.14 (2H, s), 7.41 (2H, d, J=8 Hz)

MS (ESI−): 403 (M−1)

### EXAMPLE 192

To a solution of 2-(trimethylsilyl)ethyl ((2S)-2-(5-(4-(4-aminophenyl)phenyl)thiophen-2-yl)-1,1-dioxo-3,4,5,6-tetrahydro-2H-thiopyran-2-yl)acetate (130 mg), 37% formaldehyde (460 mg) and sodium cyanoborohydride (45.2 mg) in methanol (3 ml) and acetnitrile (3 ml) was added acetic acid (5 drops) and the mixture was stirred at ambient temperature for 2 hours. The mixture was partitioned between ethyl acetate and saturated sodium bicarbonate solution. The organic layer was separated, washed with brine, dried over magnesium sulfate, and evaporated in vacuo. The residue was purified by silica gel column chromatography eluting with a mixture of hexane-ethyl acetate (3:1-1:1) to give 2-(trimethylsilyl)ethyl ((2S)-2-(5-(4-(4-dimethylaminophenyl)phenyl)thiophen-2-yl)-1,1-dioxo-3,4,5,6-tetrahydro-2H-thiopyran-2-yl)acetate as a yellow amorphous powder (102 mg).

NMR (CDCl₃, δ): −0.04 (9H, s), 0.76-0.90 (2H, m), 1.76-2.26 (4H, m), 2.68-2.95 (2H, m), 3.01 (6H, s), 3.05-3.25 (2H, m), 3.14 (1H, d, J=15 Hz), 3.43 (1H, d, J=15 Hz), 3.96-4.08 (2H, m), 6.80 (2H, d, J=8 Hz), 7.26 (2H, m), 7.47-7.64 (6H, m)

### EXAMPLE 193

To a solution of (2S)-2-[5-(4-ethoxyphenyl)thiophen-2-yl]-1,1-dioxo-3,4,5,6-tetrahydro-2H-thiopyran-2-acetaldehyde (65 mg), 2-methyl-2-butene (53 mg), and sodium dihydrogenphosphate dihydrate (27 mg) in tert-butanol (1 ml), water (1 ml), and dioxane (1 ml) was added sodium chlorite (53 mg) at ambient temperature. After 3 hours, the reaction mixture was concentrated to about ¼ volume. To the mixture was added water and was adjusted to pH3 with 1N HCl. The precipitate was filtered and was washed with water. The solid was triturated with isopropyl ether to give (2S)-2-[5-(4-ethoxyphenyl)thiophen-2-yl]-1,1-dioxo-3,4,5,6-tetrahydro-2H-thiopyran-2-acetic acid as a yellow solid (64 mg).

NMR (DMSO-d₆, δ): 1.33 (3H, t, J=8 Hz), 1.69-2.10 (4H, m), 2.43 (1H, m), 2.81 (1H, m), 3.09-3.63 (4H, m), 4.05 (2H, q, J=8 Hz), 6.96 (2H, d, J=8 Hz), 7.12 (1H, d, J=4 Hz), 7.33 (1H, d, J=4 Hz), 7.55 (2H, d, J=8 Hz)

### EXAMPLE 194

To a solution of 2-(trimethylsilyl)ethyl (2S)-2-(5-bromothiophen-2-yl)-1,1-dioxo-3,4,5,6-tetrahydro-2H-thiopyran-2-acetate (100 mg) in N,N-dimethylformamide (2 ml) was added copper(I) iodide (4 mg), palladium(II) acetate (1.5 mg), triphenylphosphine (7 mg), triethylamine (67 mg), and 1-ethynyl-4-methoxybenzene (58 mg) at ambient temperature under nitrogen gas atmosphere. The mixture was heated at 60° C. for 4 hours. The cooled reaction mixture was concentrated to about ⅓ volume. The residue was partitioned between ethyl acetate and water, and was filtered through Celite. The organic layer was separated, washed with brine, died over magnesium sulfate, and evaporated in vacuo. To the residue was added AcOEt and the precipitate was filtered off. The mother layer was concentrated and the residue was purified by flash silica gel chromatography (silica gel, 50 ml) eluting with hexane-ethyl acetate=5-1 and 2-1 to give 2-(trimethylsilyl)ethyl (2S)-2-[5-[(4-methoxyphenyl)ethynyl]thiophen-2-yl]-3,4,5,6-tetrahydro-2H-thiopyran-2-acetate as a colorless amorphous (111 mg).

NMR (CDCl₃, δ): 0.01 (9H, s), 0.77-0.90 (2H, m), 1.74-2.25 (4H, m), 2.64-2.89 (2H, m), 2.99-3.18 (3H, m), 3.47 (1H, d, J=15 Hz), 3.83 (3H, s), 3.95-4.10 (2H, m), 6.86 (2H, d, J=8 Hz), 7.16 (2H, br s), 7.41 (2H, d, J=8 Hz)

MS (ESI+): 505 (M+1)

1. A compound of the formula: 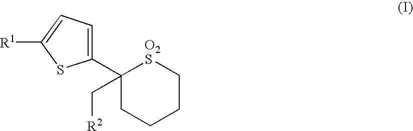
in which
R¹ is optionally substituted phenyl,
optionally substituted naphthyl,
optionally substituted bicyclic heterocyclic group
optionally substituted lower alkenyl or
optionally substituted lower alkynyl, and
R² is carboxy or protected carboxy, or a salt thereof.

2. The compound of claim 1, wherein
R¹ is phenyl optionally substituted by the group consisting of
lower alkyl,
amino,
lower alkylureido,
hydroxy,
lower alkoxy,
lower cycloalkyl,
phenyl(lower)alkoxy,
heterocyclic(lower)alkoxy,
lower alkylcarbamoyl(lower)alkenyl,
heterocyclic(lower)alkenyl,
heterocycliccarbonylamino,
heterocyclic group optionally substituted by the group consisting of lower alkyl and phenyl,
phenyl,
lower alkoxyphenyl,
lower alkylphenyl,
lower alkylthiophenyl,
cyanophenyl,
lower alkanoylphenyl,
halophenyl,
trihalo(lower)alkylphenyl,
trihalo(lower)alkanoylphenyl,
aminophenyl,
mono or di(lower)alkylaminophenyl,
lower alkoxycarbonylaminophenyl,
lower alkanoylaminophenyl,
lower alkylsulfonylaminophenyl,
lower alkylureidophenyl,
carbamoylphenyl,
mono or di(lower)alkylcarbamoylphenyl,
lower alkylsulfonylcarbamoylphenyl,
lower alkoxycarbonylphenyl,
lower alkylaminosulfonylphenyl,
hydroxyphenyl,
hydroxy(lower)alkylphenyl, and
heterocyclic-phenyl;
naphthyl optionally substituted by the group consisting of
hydroxy,
lower alkoxy,
lower alkoxy(lower)alkoxy,
cyano,
lower alkanoyl,
lower alkoxycarbonyl,
lower alkylcarbamoyl, and
heterocyclic group;
bicyclic heterocyclic group optionally substituted by the group consisting of consisting of lower alkyl, lower alkoxy, halogen and oxo;
lower alkenyl optionally substituted by C₆-C₁₀ aryl which is optionally substituted by the group consisting of
lower alkyl,
halogen,
hydroxy,
lower alkoxycarbonyloxy, and
lower alkoxy; or
lower alkynyl optionally substituted by C₆-C₁₀ aryl which is optionally substituted by the group consisting of
lower alkyl,
lower alkoxy,
halo,
cyano, and
heterocyclic group;
wherein the above heterocyclic groups are selected from the group consisting of
unsaturated 5 or 6-membered heteromonocyclic group containing 1 to 4 nitrogen atom(s),
unsaturated 5 or 6-membered heteromonocyclic group containing 1 or 2 oxygen atom(s) and 1 to 3 nitrogen atom(s),
unsaturated 3 to 8-membered (more preferably 5 or 6-membered) heteromonocyclic group containing 1 or 2 sulfur atom(s) and 1 to 3 nitrogen atom(s),
unsaturated 3 to 8-membered (more preferably 5 or 6-membered) heteromonocyclic group containing 1 or 2 sulfur atom(s),
unsaturated bicyclic 9- or 10-membered, heterocyclic group containing 1 to 5 nitrogen atoms,
unsaturated bicyclic 9- or 10-membered, heterocyclic group containing 1 or 2 oxygen atoms and 1 to 3 nitrogen atoms,
unsaturated bicyclic 9- or 10-membered, heterocyclic group containing 1 or 2 sulfur atoms and 1 to 3 nitrogen atoms,
unsaturated bicyclic 9- or 10-membered, heterocyclic group containing 1 or 2 oxygen atoms, and
unsaturated bicyclic 9- or 10-membered, heterocyclic group containing 1 or 2 sulfur atoms.

3. The compound of claim 2, wherein
R¹ is phenyl optionally substituted by the group consisting of
lower alkyl,
amino,
lower alkylureido,
hydroxy,
lower alkoxy,
lower cycloalkyl,
phenyl(lower)alkoxy,
oxazolyl(lower)alkoxy,
lower alkylcarbamoyl(lower)alkenyl,
oxazolyl(lower)alkenyl,
oxazolylcarbonylamino,
oxazolyl optionally substituted by lower alkyl,
isoxazolyl,
oxadiazolyl optionally substituted by lower alkyl,
thiazolyl optionally substituted by lower alkyl,
pyridyl,
pyrazolyl optionally substituted by lower alkyl,
pyrazinyl,
pyrimidinyl,
tetrazolyl optionally substituted by phenyl,
thienyl,
phenyl,
lower alkoxyphenyl,
lower alkylphenyl,
lower alkylthiophenyl,
cyanophenyl,
lower alkanoylphenyl,
halophenyl,
trihalo(lower)alkylphenyl,
trihalo(lower)alkanoylphenyl,
aminophenyl,
mono or di(lower)alkylaminophenyl,
lower alkoxycarbonylaminophenyl,
lower alkanoylaminophenyl,
lower alkylsulfonylaminophenyl,
lower alkylureidophenyl,
carbamoylphenyl,
mono or di(lower)alkylcarbamoylphenyl,
lower alkylsulfonylcarbamoylphenyl,
lower alkoxycarbonylphenyl,
lower alkylaminosulfonylphenyl,
hydroxyphenyl,
hydroxy(lower)alkylphenyl, and
oxazolylphenyl;
naphthyl optionally substituted by the group consisting of
hydroxy,
lower alkoxy,
lower alkoxy(lower)alkoxy,
cyano,
lower alkanoyl,
lower alkoxycarbonyl,
lower alkylcarbamoyl, and
oxazolyl;
benzofuryl;
dihydrobenzofuyl;
dioxoindanyl;
benzothienyl optionally substituted by the group consisting of lower alkyl, lower alkoxy and halogen;
quinolyl;
dihydroindolyl optionally substituted by lower alkyl and oxo;
benzoxazolyl;
dihydobenzothiazolyl;
lower alkenyl optionally substituted by C₆-C₁₀ aryl which is optionally substituted by the group consisting of
lower alkyl,
halogen,
hydroxy,
lower alkoxycarbonyloxy, and
lower alkoxy; or
lower alkynyl optionally substituted by C₆-C₁₀ aryl which is optionally substituted by the group consisting of
lower alkyl,
lower alkoxy,
halo,
cyano, and
oxazolyl; and
R² is carboxy, or a salt thereof.

4. The compound of claim 3, wherein
R¹ is phenyl optionally substituted by the group consisting of
lower alkyl,
amino,
lower alkylureido,
hydroxy,
lower alkoxy,
lower cycloalkyl,
phenyl(lower)alkoxy,
oxazolyl(lower)alkoxy,
lower alkylcarbamoyl(lower)alkenyl,
oxazolyl(lower)alkenyl,
oxazolylcarbonylamino,
oxazolyl optionally substituted by lower alkyl,
isoxazolyl,
oxadiazolyl optionally substituted by lower alkyl,
thiazolyl optionally substituted by lower alkyl,
pyridyl,
pyrazolyl optionally substituted by lower alkyl,
pyrazinyl,
pyrimidinyl,
tetrazolyl optionally substituted by phenyl,
thienyl,
phenyl,
lower alkoxyphenyl,
lower alkylphenyl,
lower alkylthiophenyl,
cyanophenyl,
lower alkanoylphenyl,
halophenyl,
trihalo(lower)alkylphenyl,
trihalo(lower)alkanoylphenyl,
aminophenyl,
mono or di(lower)alkylaminophenyl,
lower alkoxycarbonylaminophenyl,
lower alkanoylaminophenyl,
lower alkylsulfonylaminophenyl,
lower alkylureidophenyl,
carbamoylphenyl,
mono or di(lower)alkylcarbamoylphenyl,
lower alkylsulfonylcarbamoylphenyl,
lower alkoxycarbonylphenyl,
lower alkylaminosulfonylphenyl,
hydroxyphenyl,
hydroxy(lower)alkylphenyl, and
oxazolylphenyl.

5. The compound of claim 4, wherein
R¹ is
lower alkylphenyl,
aminophenyl,
lower alkylureidophenyl,
hydroxyphenyl,
lower alkoxyphenyl,
lower cycloalkylphenyl,
phenyl(lower)alkoxyphenyl,
oxazolyl(lower)alkoxyphenyl,
lower alkylcarbamoyl(lower)alkenylphenyl,
oxazolyl(lower)alkenylphenyl,
oxazolylcarbonylaminophenyl,
oxazolylphenyl optionally substituted by lower alkyl,
phenyl substituted by oxazolyl and lower alkyl,
isoxazolylphenyl,
oxadiazolylphenyl optionally substituted by lower alkyl,
thiazolylphenyl optionally substituted by lower alkyl,
pyridylphenyl,
pyrazolylphenyl optionally substituted by lower alkyl,
pyrazinylphenyl,
pyrimidinylphenyl,
tetrazolylphenyl optionally substituted by phenyl,
thienylphenyl,
phenylphenyl,
lower alkoxyphenylphenyl,
lower alkylphenylphenyl,
lower alkylthiophenylphenyl,
cyanophenylphenyl,
lower alkanoylphenylphenyl,
halophenylphenyl,
trihalo(lower)alkylphenylphenyl,
trihalo(lower)alkanoylphenylphenyl,
aminophenylphenyl,
mono or di(lower)alkylaminophenylphenyl,
lower alkoxycarbonylaminophenylphenyl,
lower alkanoylaminophenylphenyl,
lower alkylsulfonylaminophenylphenyl,
lower alkylureidophenylphenyl,
carbamoylphenylphenyl,
mono or
di(lower)alkylcarbamoylphenylphenyl,
lower alkylsulfonylcarbamoylphenylphenyl,
lower alkoxycarbonylphenylphenyl,
lower alkylaminosulfonylphenylphenyl,
hydroxyphenylphenyl,
hydroxy(lower)alkylphenylphenyl, and
oxazolylphenylphenyl.

6. The compound of claim 5, wherein
R¹ is 4-(n-propyl)phenyl, 4-(n-butyl)phenyl,
4-(n-pentyl)phenyl,
4-aminophenyl,
4-(n-propylureido) phenyl,
4-hydroxyphenyl,
4-ethoxyphenyl, 4-(n-propoxy)phenyl,
4-(i-propoxy)phenyl, 4-(butoxy)phenyl,
4-(n-pentyloxy)phenyl,
4-cyclohexylphenyl,
4-benzyloxyphenyl,
4-(5-oxazolylmethoxy)phenyl,
4-(2-methylcarbamoylethenyl)phenyl,
4-(2-(oxazol-5-yl)ethenyl)phenyl,
4-(oxazol-5-ylcarbonylamino)phenyl,
4-(2- or 4- or 5-oxazolyl)phenyl,
4-(2- or 4-methyl-5-oxazolyl)phenyl,
3-methyl-4-(5-oxazolyl)phenyl,
4-(5-isoxazolyl)phenyl,
4-(5-methyl-1,2,4-oxadiazol-3-yl)phenyl, 4-(2- or
4-thiazolyl)phenyl,
2-methyl-4-thiazolylphenyl,
4-(2- or 3- or 4-pyridyl)phenyl,
4-(1-methyl-5-pyrazoyl)phenyl,
4-(2-pyrazinyl)phenyl,
2-(5-pyrimidinyl)phenyl,
4-(2-phenyl-1,2,3,4,-tetrazol-5-yl)phenyl,
4-(2- or 3-thienyl)phenyl,
4-phenylphenyl,
4-(4-methoxyphenyl)phenyl,
4-(4-ethoxyphenyl)phenyl,
4-(2- or 3- or 4-methylphenyl)phenyl,
4-(4-ethylphenyl)phenyl,
4-(4-butylphenyl)phenyl,
4-(4-methylthiophenyl)phenyl,
4-(3- or 4-cyanophenyl)phenyl,
4-(4-acetylphenyl)phenyl,
4-(4-chlorophenyl)phenyl,
4-(4-fluorophenyl)phenyl,
4-(4-trifluoromethylphenyl)phenyl,
4-(4-trifluoroacetylphenyl)phenyl,
4-(4-aminophenyl)phenyl,
4-(4-dimethylaminophenyl)phenyl,
4-(4-methoxycarbonylaminophenyl)phenyl,
4-(4-t-butoxycarbonylaminophenyl)phenyl,
4-(4-acetylaminophenyl)phenyl,
4-(4-methylsulfonylaminophenyl)phenyl,
4-(4-ethylureidophenyl)phenyl,
4-(4-carbamoylphenyl)phenyl,
4-(4-methylcarbamoylphenyl)phenyl,
4-(4-dimethylcarbamoylphenyl)phenyl,
4-(4-methylsulfonylcarbamoylphenyl)phenyl,
4-(4-methoxycarbonylphnenyl)phenyl,
4-(4-methylaminosulfonylphenyl)phenyl,
4-(4-hydroxyphenyl)phenyl,
4-(4-hydroxymethylphenyl)phenyl, and
4-(4-(5-oxazolyl)phenyl)phenyl.

7. The compound of claim 3, wherein
R¹ is phenyl optionally substituted by the group consisting of
phenyl,
lower alkoxyphenyl,
lower alkylphenyl,
lower alkylthiophenyl,
cyanophenyl,
lower alkanoylphenyl,
halophenyl,
trihalo(lower)alkylphenyl,
trihalo(lower)alkanoylphenyl,
aminophenyl,
mono or di(lower)alkylaminophenyl,
lower alkoxycarbonylaminophenyl,
lower alkanoylaminophenyl,
lower alkylsulfonylaminophenyl,
lower alkylureidophenyl,
carbamoylphenyl,
mono or di(lower)alkylcarbamoylphenyl,
lower alkylsulfonylcarbamoylphenyl,
lower alkoxycarbonylphenyl,
lower alkylaminosulfonylphenyl,
hydroxyphenyl,
hydroxy(lower)alkylphenyl, and oxazolylphenyl.

8. The compound of claim 7, wherein
R¹ is phenylphenyl,
lower alkoxyphenylphenyl,
lower alkylphenylphenyl,
lower alkylthiophenylphenyl,
cyanophenylphenyl,
lower alkanoylphenylphenyl,
halophenylphenyl,
trihalo(lower)alkylphenylphenyl,
trihalo(lower)alkanoylphenylphenyl,
aminophenylphenyl,
mono or di(lower)alkylaminophenylphenyl,
lower alkoxycarbonylaminophenylphenyl,
lower alkanoylaminophenylphenyl,
lower alkylsulfonylaminophenylphenyl,
lower alkylureidophenylphenyl,
carbamoylphenylphenyl,
mono or di(lower)alkylcarbamoylphenylphenyl,
lower alkylsulfonylcarbamoylphenylphenyl,
lower alkoxycarbonylphenylphenyl,
lower alkylaminosulfonylphenylphenyl,
hydroxyphenylphenyl,
hydroxy(lower)alkylphenylphenyl, or
oxazolylphenylphenyl.

9. The compound of claim 3, wherein
R¹ is phenyl optionally substituted by the group consisting of
lower alkyl,
amino,
lower alkylureido,
hydroxy,
lower alkoxy,
lower cycloalkyl,
phenyl(lower)alkoxy,
oxazolyl(lower)alkoxy,
lower alkylcarbamoyl(lower)alkenyl,
oxazolyl(lower)alkenyl,
oxazolylcarbonylamino,
oxazolyl optionally substituted by lower alkyl,
isoxazolyl,
oxadiazolyl optionally substituted by lower alkyl,
thiazolyl optionally substituted by lower alkyl,
pyridyl,
pyrazolyl optionally substituted by lower alkyl,
pyrazinyl,
pyrimidinyl,
tetrazolyl optionally substituted by phenyl, and thienyl.

10. The compound of claim 9, wherein
R¹ is lower alkylphenyl,
aminophenyl,
lower alkylureidophenyl,
hydroxyphenyl,
lower alkoxyphenyl,
lower cycloalkylphenyl,
phenyl(lower)alkoxyphenyl,
oxazolyl(lower)alkoxyphenyl,
lower alkylcarbamoyl(lower)alkenylphenyl,
oxazolyl(lower)alkenylphenyl,
oxazolylcarbonylaminophenyl,
oxazolylphenyl optionally substituted by lower alkyl,
phenyl substituted by oxazolyl and lower alkyl,
isoxazolylphenyl,
oxadiazolylphenyl optionally substituted by lower alkyl,
thiazolylphenyl optionally substituted by lower alkyl,
pyridylphenyl,
pyrazolylphenyl optionally substituted by lower alkyl,
pyrazinylphenyl,
pyrimidinylphenyl, tetrazolylphenyl optionally substituted by phenyl, or
thienylphenyl.

11. The compound of claim 10, wherein
R¹ is 4-(2- or 4- or 5-oxazolyl)phenyl, 4-(2- or
4-methyl-5-oxazolyl)phenyl,
4-(oxazol-5-yl)-3-methylphenyl, 4-(5-isoxazolyl phenyl,
4-(5-methyl-1,2,4-oxadiazol-3-yl)phenyl,
4-(2- or 4-thiazolyl)phenyl,
2-methyl-4-thiazolylphenyl,
4-(2- or 3- or 4-pyridyl)phenyl,
4-(1-methyl-5-pyrazolyl)phenyl,
4-(2-pyrazinyl)phenyl,
2-(5-pyrimidinyl)phenyl,
4-(2-phenyl-1,2,3,4,-tetrazol-5-yl)phenyl, or
4-(2- or 3-thienyl)phenyl).

12. The compound of claim 3, wherein
R¹ is naphthyl optionally substituted by the group consisting of
hydroxy,
lower alkoxy,
lower alkoxy(lower)alkoxy,
cyano,
lower alkanoyl,
lower alkoxycarbonyl,
lower alkylcarbamoyl, and
oxazolylnaphthyl.

13. The compound of claim 12, wherein
R¹ is naphthyl
hydroxynaphthyl,
lower alkoxynaphthyl,
lower alkoxy(lower)alkoxynaphthyl,
cyanonaphthyl,
lower alkanoylnaphthyl,
lower alkoxycarbonylnaphthyl,
lower alkylcarbamoylnaphthyl, or
oxazolylnaphthyl.

14. The compound of claim 13, wherein
R¹ is 2-naphthyl,
6-hydroxy-2-naphthyl,
6-methoxy2-naphthyl, 6-ethoxy-2-naphthyl,
6-methoxymethoxy-2-naphthyl,
6-cyano-2-naphthyl,
6-formyl-2-naphthyl,
6-methoxycarbonyl-2-naphthyl,
6-methylcarbamoyl-2-naphthyl, or
6-(5-oxazolyl)-2-naphthyl.

15. The compound of claim 3, wherein
R¹ is benzofuryl;
dihydrobenzofuyl;
dioxoindanyl;
benzothienyl optionally substituted by the group consisting of lower alkyl, lower alkoxy and halogen;
quinolyl;
dihydroindolyl optionally substituted by lower alkyl and oxo;
benzoxazolyl;
dihydobenzothiazolyl.

16. The compound of claim 15, wherein
R¹ is benzofuryl,
dihydrobenzofuyl,
dioxoindanyl,
benzothienyl,
lower alkylbenzothienyl,
lower alkoxybenzothienyl,
halobenzothienyl,
quinolyl,
dihydroindolyl optionally substituted by lower alkyl and oxo,
benzoxazolyl, or
dihydobenzothiazolyl.

17. The compound of claim 16, wherein
R¹ is 2- or 5-benzofuryl,
2,3-dihydro-5-benzofuryl;
1,3-dioxoindan-5-yl,
2- or 3- or 5-benzothienyl,
5-methyl-2-benzothienyl,
5- or 6-methoxy-2-benzothienyl,
5- or 6-fluoro-2-benzothienyl,
3- or 6-quinolyl,
1-mehtyl-2-oxo-2,3-dihydroindol-5-yl, benzoxazol-2-yl, or
3-methyl-2-oxo-2,3-dihydrobenzothiazol-5-yl.

18. The compound of claim 3, wherein
R¹ is lower alkenyl optionally substituted by C₆-C₁₀ aryl
which is optionally substituted by the group consisting of
lower alkyl,
halogen,
hydroxy,
lower alkoxycarbonyloxy, and
lower alkoxy.

19. The compound of claim 18, wherein
R¹ is phenyl(lower)alkenyl,
lower alkylphenyl(lower)alkenyl,
halophenyl(lower)alkenyl,
hydroxyphenyl(lower)alkenyl,
lower alkoxycarbonyloxyphenyl(lower)alkenyl,
lower alkoxyphenyl(lower)alkenyl, or
lower alkoxycarbonyloxyphenyl(lower)alkenyl.

20. The compound of claim 19, wherein
R¹ is 2-phenylethenyl, 2-(2-naphthyl)ethenyl,
2-(4-methylphenyl)ethenyl,
2-(4-chlorophenyl)ethenyl,
2-(4-fluorophenyl)ethenyl,
2-(4-hydroxyphenyl)ethenyl,
2-(4-methoxyphenyl)ethenyl, or
2-(4-t-butoxycarbonyloxyphenyl)ethenyl.

21. The compound of claim 3, wherein
R¹ is lower alkynyl optionally substituted by
C₆-C₁₀ aryl which is optionally substituted by the group consisting of
lower alkyl, lower alkoxy,
halo,
cyano, and
oxazolyl.

22. The compound of claim 21, wherein
R¹ is lower alkynyl,
Phenyl(lower)alkynyl,
lower alkylphenyl(lower)alkynyl,
lower alkoxyphenyl(lower)alkynyl,
halophenyl(lower)alkynyl,
cyanophenyl(lower)alkynyl, or
oxazolylphenyl(lower)alkynyl.

23. The compound of claim 22, wherein
R¹ is 1-pentynyl,
phenylethynyl,
4-methylphenylethynyl,
4-methoxyphenylethynyl, 4-ethoxyphenylethynyl,
4-chlorophenylethynyl,
4-cyanophenylethynyl, or
4-(oxazol-5-yl)phenylethynyl.

24. A process for the preparation of a compound of the formula: 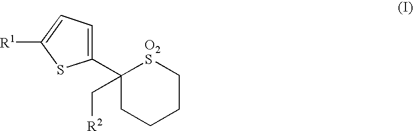
which comprises
(1) subjecting a compound of the formula 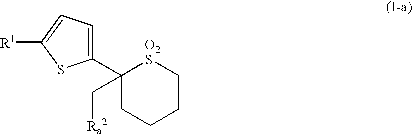
or a salt thereof to a removal reaction of the carboxy-protective group, to give a compound of the formula: 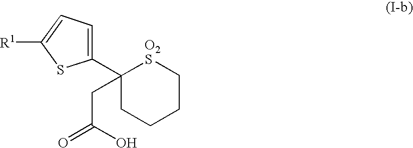
or a salt thereof; or
(2) reacting a compound of the formula 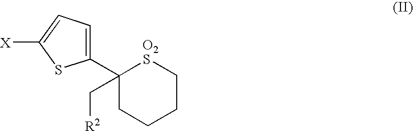
or a salt thereof, with a compound of the formula: 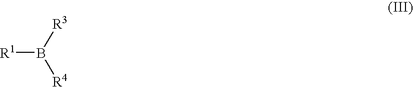
to give a compound of the above formula (I) or a salt thereof; or
(3) reacting a compound of the formula: 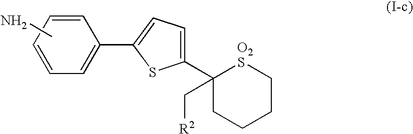
or a salt thereof, with a compound of the formula:
R⁵—NCO (IV)
to give a compound of the formula: 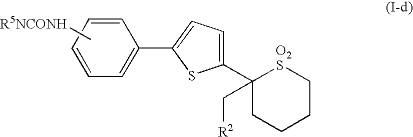
or a salt thereof; or
(4) reacting a compound of the formula: 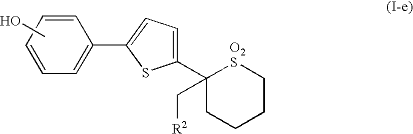
or a salt thereof, with a compound of the formula:
R⁵—X (V)
to give a compound of the formula: 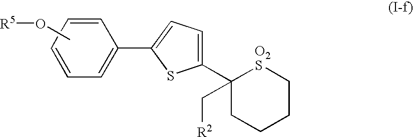
or a salt thereof; or
(5) reacting a compound of the formula: 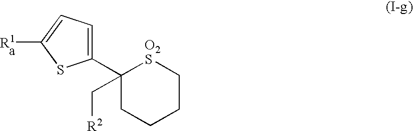
or a salt thereof to a removal reaction of the amino-protective group to give a compound of the formula: 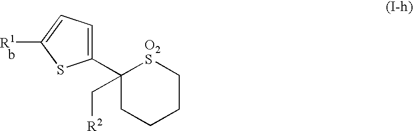
or a salt thereof; or
(6) reacting a compound of the formula: 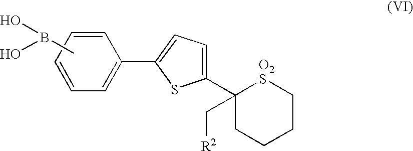
or a salt thereof, with a compound of the formula:
R⁶—X (VII)
to give a compound of the formula: 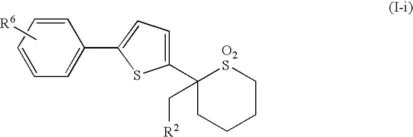
or a salt thereof; or
(7) subjecting a compound of the formula: 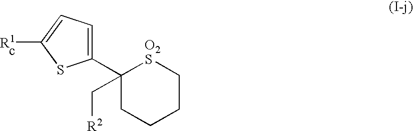
or a salt thereof, to a removal reaction for the hydroxy-protective group to give a compound of the formula: 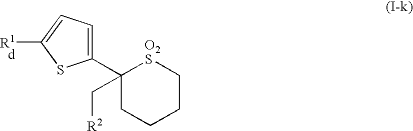
or a salt thereof; or
(8) reacting a compound of the formula: 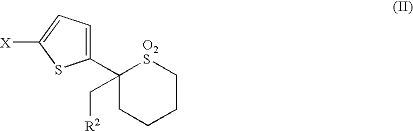
or a salt thereof, with optionally substituted lower alkyne
to give a compound of the formula: 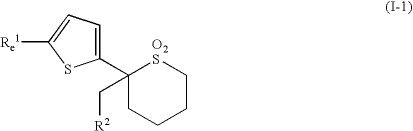
or a salt thereof; or
(9) reacting a compound of the formula: 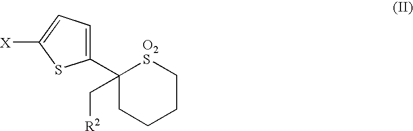
or a salt thereof, with optionally substituted lower alkene to give a compound of the formula: 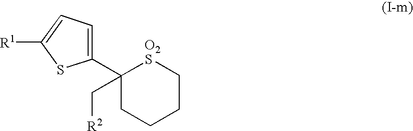
or a salt thereof; or
(10) reacting a compound of the formula: 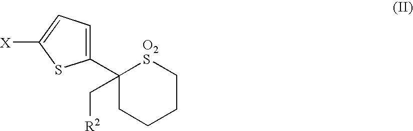
or a salt thereof, with a compound of the formula: 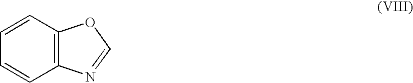
to give a compound of the formula: 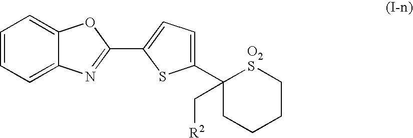
or a salt thereof; or
(11) reacting a compound of the formula: 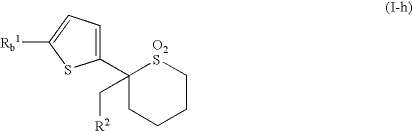
or a salt thereof, with lower alkanoic anhydride to give a compound of the formula: 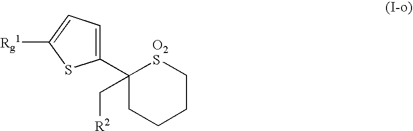
or a salt thereof; or
(12) alkylating a compound of the formula: 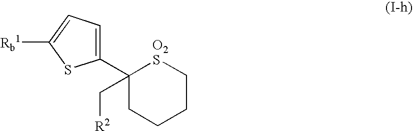
or a salt thereof, to give a compound of the formula: 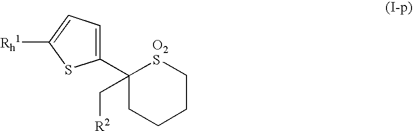
or a salt thereof; or
(13) oxydating a compound of the formula: 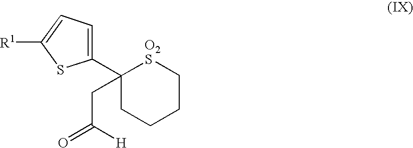
or a salt thereof, to give a compound of the formula: 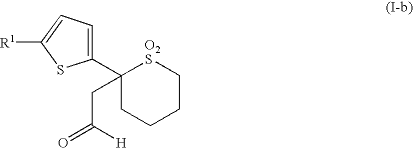
or a salt thereof;
in which R¹ and R² are each as defined in claim 1,
Rₐ¹ is above-mentioned R¹ which has
a protected amino moiety such as lower
alkoxycarbonylamino or lower alkanoyl amino moiety,
R_{b}¹ is above-mentioned R¹ which has
an amino moiety,
R_{c}¹ is above-mentioned R¹ which has
a protected hydroxy moiety such as lower alkoxycarbonyloxy moiety,
R_{d}¹ is above-mentioned R¹ which has
a hydroxy moiety,
Rₑ¹ is optionally substituted lower alkynyl,
R_{f}¹ is optionally substituted lower alkenyl,
R_{g}¹ is above-mentioned R¹ which has
an lower alkanoylamino moiety,
Rₕ¹ is above-mentioned R¹ which has
an mono- or di(lower)alkylamino moiety,
Rₐ² is protected carboxy,
R³ and R⁴ are each hydroxy, lower alkyl, or combined together to form lower alkylene,
R⁵ is lower alkyl,
R⁶ is suitable substituent, and
X is a leaving group.

25. A pharmaceutical composition which comprises the compound of claim 1 or a pharmaceutically acceptable salt thereof and a pharmaceutically acceptable carrier or excipient.

26. A process for preparing a pharmaceutical composition which comprises admixing the compound of claim 1 or a pharmaceutically acceptable salt thereof with a pharmaceutically acceptable carrier or excipient.

27. Use of the compound of claim 1 or a pharmaceutically acceptable salt thereof as a medicament.

28. Use of the compound of claim 1 or a pharmaceutically acceptable salt thereof as an inhibitor of matrix metalloproteinases (MMP) or tumor necrosis factor α (TNF α).

29. Use of the compound of claim 1 or a pharmaceutically acceptable salt thereof for manufacturing a medicament for treating and/or preventing MMP- or TNF α-mediated diseases.

30. A method for treating and/or preventing MMP- or TNF α-mediated diseases which comprises administering the compound of claim 1 or a pharmaceutically acceptable salt thereof to a human being or an animal.

31. Use of the compound of claim 1 or a pharmaceutically acceptable salt thereof for treating and/or preventing MMP- or TNF α-mediated diseases.
